(19) United States
(12) Patent Application Publication
(10) Pub. No.: US 2011/0002897 A1
(43) Pub. Date: Jan. 6, 2011

(54) DIRECTED DIFFERENTIATION OF STEM CELLS

(75) Inventors: Evan Snyder, San Diego, CA (US); Ilyas Singec, San Diego, CA (US)

Correspondence Address: PATENT CORRESPONDENCE;ARNALL GOLDEN GREGORY LLP, 171 17TH STREET NW, SUITE 2100, ATLANTA, GA, 30363, US

(73) Assignee: Burnham Institute for Medical Research, La Jolla, CA (US)

(21) Appl. No.: 12/813,174

(22) Filed: Jun. 10, 2010

Related U.S. Application Data
(60) Provisional application No. 61/186,348, filed on Jun. 11, 2009.

(51) Int. Cl. A61K 35/30; C12N 5/00; C12N 5/079; C12Q 1/02; A61P 25/00

(52) U.S. Cl. 424/93.7; 435/377; 435/368; 435/29

(57) ABSTRACT

Disclosed are compositions and methods for producing neural cells from stem cells and uses thereof.

### CROSS-REFERENCE TO RELATED APPLICATIONS

This application claims benefit of U.S. Provisional Application No. 61/186,348, filed Jun. 11, 2009. Application No. 61/186,348, filed Jun. 11, 2009, is hereby incorporated herein by reference in its entirety.

### STATEMENT REGARDING FEDERALLY SPONSORED RESEARCH

This invention was made with government support under Grant Nos. P20-GM075059 and PO1ES016738-01 awarded by the National Institutes of Health (NIH). The government has certain rights in the invention.

### BACKGROUND

The first human embryonic stem cell (hESC) lines were established in 1998 by James Thomson and colleagues. However, even after one decade of work with hESCs the practical use of these cells is very limited because of a poor knowledge of the molecular mechanisms that control their differentiation. Specifically, the earliest differentiation step from a pluripotent hESC to a neuroectodermal cell type, also called neural induction, has been poorly understood. So far, the production of neural cells from hESCs has solely been based on empirical methods such as embryoid body formation, co-culture with murine stromal cells, and overgrowth of hESC cultures in order to obtain spontaneously differentiating neural cells. It is widely accepted in the stem cell field that these methods are insufficient, time-consuming (neural induction on murine stromal cells can take up to 4 weeks), and generate a heterogeneous population of cells including mesodermal, endodermal, and extra-embryonic lineages.

The development of an efficient neural induction protocol that allows the controlled and directed production of unlimited numbers of human neural stem cell is of paramount importance for basic research and future clinical cell therapy. Clearly, the availability of an efficient neural induction protocol would allow systematic and rational strategies to generate the three major cell types of the central nervous system (i.e. various neuronal cell types such as cortical pyramidal neurons, dopamine neurons and motorneurons, astrocytes, and oligodendrocytes).

### SUMMARY

Disclosed are compositions and methods for producing neural cells from stem cells. For example, disclosed are methods involving generation of neural stem cells from pluripotent stem cells. Also disclosed are neural cells and neural stem cells produced in the disclosed methods. Also disclosed are compositions and methods of using neural cells and neural stem cells produced in the disclosed methods. Also disclosed are compositions and methods of treating a subject using the neural cells and neural stem cells produced in the disclosed methods. Also disclosed are compositions and methods of detecting a state or characteristic of neural cells and neural stem cells produced in the disclosed methods. Also disclosed are compositions and methods testing conditions for differentiation of neural stem cells using the neural cells and neural stem cells produced in the disclosed methods. Also disclosed are methods of producing differentiated neural cells using the neural cells and neural stem cells produced in the disclosed methods.

For example, disclosed are methods of producing neural cells, the method comprising incubating pluripotent stem cells in the presence of an amount of one or more Neural Development Factors (NDF) sufficient to generate cells expressing Pax-6, Otx2, Nestin, or a combination, thereby producing neural cells. Also disclosed are neural cells produced by one or more of the disclosed methods. Also disclosed are neural cells produced by the method comprising incubating pluripotent stem cells in the presence of an amount of one or more Neural Development Factors (NDF) sufficient to generate cells expressing Pax-6, Otx2, Nestin, or a combination.

Also disclosed are methods of treating a subject, the method comprising administering a neural cell produced by one or more of the disclosed methods. Also disclosed are methods of treating a subject, the method comprising incubating pluripotent stem cells in the presence of an amount of one or more Neural Development Factors (NDF) sufficient to generate cells expressing Pax-6, Otx2, Nestin, or a combination, thereby producing neural cells, and administering one or more of the neural cells to the subject. The stem cell can be, for example, from the same species as the subject. The stem cell can be, for example, from the subject.

Also disclosed are methods of detecting a state or characteristic of a cell, the method comprising detecting the state or characteristic in a neural cell produced by one or more of the disclosed methods. Also disclosed are methods of testing conditions for differentiation of neural stem cells, the method comprising exposing a neural cell produced by one or more of the disclosed methods to test conditions and determining if the neural stem cells differentiate into a cell type of interest. The cell type of interest can be, for example, neuron, astrocyte, oligodendrocyte, or a combination.

Also disclosed are methods of producing differentiated neural cells, the method comprising incubating pluripotent stem cells in the presence of an amount of one or more Neural Development Factors (NDF) sufficient to generate cells expressing Pax-6, Otx2, Nestin, or a combination, thereby producing neural cells, and differentiating the neural cells into differentiated neural cells. The neural cells can be differentiated into differentiated neural cells by, for example, incubating the neural cells under differentiation conditions. The differentiation conditions can be chosen based on the type of differentiated neural cells desired or sought.

Also disclosed are methods of producing neural cells, the method comprising incubating pluripotent stem cells in the presence of an amount of one or more Neural Development Factors (NDF) sufficient to generate cells expressing Pax-6, Otx2, Nestin, or a combination, thereby producing neural cells, wherein the NDF activate the phosphatidylinositol 3-kinase (PI3K) signaling pathway, inhibit transforming growth factor-β (TGF-β) superfamily signaling pathway, and inhibit the Wnt signaling pathway, wherein the NDF comprise Midkine, Pleiotrophin, insulin-like growth factor-1 (IGF-1), or a combination; A83-01, SB431542, or a combination; dorsopmorphin; and PNU-74654, Dickkopf, or a combination.

Also disclosed are methods of producing neural cells, the method comprising (a) culturing pluripotent stem cells in the presence of fibroblast growth factor 2 (FGF-2); (b) incubating the stem cells in the presence of an amount of one or more Neural Development Factors (NDF) sufficient to generate cells expressing Pax-6, Otx2, Nestin, or a combination, thereby producing neural cells, wherein the NDF activate the phosphatidylinositol 3-kinase signaling pathway, inhibit the TGF-β superfamily signaling pathway, and inhibit the Wnt signaling pathway, wherein the NDF comprise Midkine, Pleiotrophin, insulin-like growth factor-1, or a combination; A83-01, SB431542, or a combination; dorsopmorphin; and PNU-74654, Dickkopf, or a combination, wherein prior to incubating in the presence of the NDF, at the same time as incubating in the presence of the NDF is initiated, or during incubating in the presence the NDF the use of FGF-2 is discontinued; and (c) culturing the neural cells, wherein the neural cells are cultured on a treated polymer substrate, wherein the neural cells are cultured in serum free conditions, wherein the neural cells are cultured in the presence of fibroblast growth factor 2.

Also disclosed are methods of producing neural cells, the method comprising (a) culturing pluripotent stem cells in the presence of fibroblast growth factor 2; (b) incubating the stem cells in the presence of an amount of one or more Neural Development Factors (NDF) sufficient to generate cells expressing Pax-6, Otx2, Nestin, or a combination, thereby producing neural cells, wherein the NDF activate the phosphatidylinositol 3-kinase signaling pathway, activate the Notch signaling pathway, inhibit the TGF-β superfamily signaling pathway, and inhibit the Wnt signaling pathway, wherein the NDF comprise Midkine, Pleiotrophin, insulin-like growth factor-1, or a combination; Delta-1, Delta-2, Delta-3, Delta-4, Jagged-1, Jagged-2, or a combination; A83-01, SB431542, or a combination; dorsopmorphin; and PNU-74654, Dickkopf, or a combination, wherein prior to incubating in the presence of the NDF, at the same time as incubating in the presence of the NDF is initiated, or during incubating in the presence the NDF the use of FGF-2 is discontinued; and (c) culturing the neural cells, wherein the neural cells are cultured on a treated polymer substrate, wherein the neural cells are cultured in serum free conditions, wherein the neural cells are cultured in the presence of fibroblast growth factor 2.

The treated polymer substrate can be, for example, CELLBIND™, or substrate treated with Matrigel™, Geltrex™, or fibronectin. The serum free conditions can comprise N2 supplement and B27 supplement. The neural cells can be cultured in the presence of epidermal growth factor (EGF). The neural cells can be cryopreservable. The stem can also be cultured in the presence of conditioned media or chemically defined media such as mouse embryonic fibroblast-conditioned media (MEF-CM), mTeSR™ or StemPro™. The stem cells can also be incubated in the presence of 20% knockout serum replacement (KSR).

The disclosed methods for producing neural cells from stem cells can involve incubation with NDF. The methods can further comprise pre-incubation of the stem cells prior to the incubation. The methods can further comprise culturing the neural cells produced by the incubation. The methods can further comprise a transition from pre-incubation and incubation. The methods can further comprise a transition from incubation and culturing. The methods can further provide further differentiation of the neural cells during or after culturing. For example, neural stem cells produced during incubation can be differentiated into neural cell lineages, tissue types and/or cell types during or after culturing.

The NDF can, for example, activate the phosphatidylinositol 3-kinase (PI3K) signaling pathway. The NDF can, for example, activate the mitogen-activated protein kinase (MAPK) signaling pathway. The NDF can, for example, activate the phosphatidylinositol 3-kinase signaling pathway and can activate the MAPK signaling pathway. The NDF can, for example, activate the phosphatidylinositol 3-kinase signaling pathway and the MAPK signaling pathway. The NDF can, for example, activate the phosphatidylinositol 3-kinase signaling pathway and the MAPK signaling pathway in a balanced manner. The NDF can, for example, inhibit the TGF-β superfamily signaling pathway. The NDF can, for example, inhibit the Wnt signaling pathway. The NDF can, for example, activate the phosphatidylinositol 3-kinase signaling pathway, inhibit the TGF-β superfamily signaling pathway, and inhibit the Wnt signaling pathway. The NDF can, for example, activate the phosphatidylinositol 3-kinase signaling pathway, inhibit the TGF-β superfamily signaling pathway, inhibit the Wnt signaling pathway, or any combination of these. The NDF can, for example, activate the phosphatidylinositol 3-kinase signaling pathway, activate the MAPK signaling pathway, inhibit the TGF-β superfamily signaling pathway, and inhibit the Wnt signaling pathway. The Wnt signaling pathway can be, for example, inhibited after the β-catenin destruction complex. The NDF can, for example, activate the Notch signaling pathway. The NDF can, for example, activate the phosphatidylinositol 3-kinase signaling pathway, activate the Notch signaling pathway, inhibit the TGF-β superfamily signaling pathway, and inhibit the Wnt signaling pathway. The NDF can, for example, activate the phosphatidylinositol 3-kinase signaling pathway, activate the Notch signaling pathway, inhibit the TGF-β superfamily signaling pathway, inhibit the Wnt signaling pathway, or any combination of these.

The NDF can comprise, for example, an activator of the phosphatidylinositol 3-kinase signaling pathway. The NDF can comprise, for example, an activator of the MAPK signaling pathway. The NDF can comprise, for example, an activator of the phosphatidylinositol 3-kinase signaling pathway and the MAPK signaling pathway. The NDF can comprise, for example, Midkine, Pleiotrophin, insulin-like growth factor-1, or a combination. The NDF can comprise, for example, Midkine, insulin-like growth factor-1, or a combination. The NDF can comprise, for example, Midkine. The NDF can comprise, for example, an activator of the phosphatidylinositol 3-kinase signaling pathway and an activator of the MAPK signaling pathway. The NDF can comprise, for example, an inhibitor of the TGF-(i superfamily signaling pathway. The NDF can comprise, for example, A83-01, SB431542, or a combination. The NDF can comprise, for example, dorsomorphin. The NDF can comprise, for example, dorsomorphin and A83-01. The NDF can comprise, for example, an inhibitor of the Wnt signaling pathway. The NDF can comprise, for example, PNU-74654, Dickkopf, or a combination. The NDF can comprise, for example, an activator of the phosphatidylinositol 3-kinase signaling pathway, an inhibitor of the TGF-β superfamily signaling pathway, and an inhibitor of the Wnt signaling pathway. The NDF can comprise, for example, an activator of the phosphatidylinositol 3-kinase signaling pathway, an inhibitor of the TGF-β superfamily signaling pathway, an inhibitor of the Wnt signaling pathway, or any combination of these. The NDF can comprise, for example, Midkine, Pleiotrophin, insulin-like growth factor-1, or a combination; A83-01, SB431542, or a combination; dorsopmorphin; and PNU-74654, Dickkopf, or a combination. The NDF can comprise, for example, Midkine, Pleiotrophin, insulin-like growth factor-1, or a combination; A83-01, SB431542, or a combination; dorsopmorphin; PNU-74654, Dickkopf, or a combination; or any combination of these. The NDF can comprise, for example, Midkine, A83-01, dorsopmorphin, and PNU-74654. The NDF can comprise, for example, insulin-like growth factor-1, A83-01, dorsopmorphin, and PNU-74654.

The NDF can comprise, for example, an activator of the Notch signaling pathway. The NDF can comprise, for example, Delta-1, Delta-2, Delta-3, Delta-4, Jagged-1, Jagged-2, or a combination. The NDF can comprise, for example, an activator of the phosphatidylinositol 3-kinase signaling pathway, an activator of the Notch signaling pathway, an inhibitor of the TGF-β superfamily signaling pathway, and an inhibitor of the Wnt signaling pathway. The NDF can comprise, for example, an activator of the phosphatidylinositol 3-kinase signaling pathway, an activator of the Notch signaling pathway, an inhibitor of the TGF-β superfamily signaling pathway, an inhibitor of the Wnt signaling pathway, or any combination of these. The NDF can comprise, for example, Midkine, Pleiotrophin, insulin-like growth factor-1, or a combination; Delta-1, Delta-2, Delta-3, Delta-4, Jagged-1, Jagged-2, or a combination; A83-01, SB431542, or a combination; dorsopmorphin; and PNU-74654, Dickkopf, or a combination. The NDF can comprise, for example, Midkine, Pleiotrophin, insulin-like growth factor-1, or a combination; Delta-1, Delta-2, Delta-3, Delta-4, Jagged-1, Jagged-2, or a combination; A83-01, SB431542, or a combination; dorsopmorphin; PNU-74654, Dickkopf, or a combination; or any combination of these.

The NDF can, for example, activate the Notch signaling pathway. The NDF can comprise, for example, Delta-1, Delta-2, Delta-3, Delta-4, Jagged-1, Jagged-2, or a combination. The NDF can, for example, activate the protein kinase signaling pathway. The NDF can comprise, for example, Forskolin, dibutyryl cAMP, or a combination. The NDF can, for example, activate tyrosine kinase anaplastic lymphoma kinase (ALK). The NDF can, for example, activate insulin-like growth factor (IGF) receptor. The NDF can, for example, activate phosphatidylinositol 3-kinase. The NDF can, for example, inhibit Activin receptor-like kinase 5 (ALK5). The NDF can, for example, inhibit Activin receptor-like kinase 4 (ALK4). The NDF can, for example, inhibit Activin receptor-like kinase 7 (ALK7). The NDF can, for example, inhibit ALK5, ALK4, and ALK7. The NDF can, for example, inhibit protein phosphatase 2A (PP2A). The NDF can, for example, inhibit adenosine monophosphate-activated protein kinase (AMPK). The NDF can, for example, inhibit Bone morphogenic protein (BMP) receptor. The NDF can, for example, inhibit interaction between β-catenin and T cell factor (TCF). The NDF can, for example, activate protein-tyrosine phosphatasζ (PTPζ). The NDF can, for example, inhibit SMAD2, SMAD3, SMAD4, or a combination. The NDF can, for example, activate Notch. The NDF can, for example, inhibit SMAD1, SMAD5, SMAD8, or a combination. The NDF can, for example, inhibit Wnt binding to Frizzled. The NDF can, for example, inhibit lipoprotein receptor-related protein (LRP) binding to Frizzled. The NDF can, for example, inhibit β-catenin stabilization. The NDF can, for example, inhibit β-catenin binding to T cell factor (TCT). The NDF can, for example, activate insulin-like growth factor-1 receptor (IGF-1R). The NDF can, for example, activate insulin receptor substrate-1 (IRS-1).

The methods can be performed wherein prior to incubating in the presence of the NDF, the stem cells are cultured in the absence of feeder cells and on an extracellular matrix. The extracellular matrix can be, for example, Matrigel™ or Geltrex™. The methods can be performed wherein prior to incubating in the presence of the NDF the stem cells are cultured on fibroblasts. The fibroblasts can be, for example, from the same species as the stem cells. The fibroblasts can be, for example, from the same subject as the stem cells. The fibroblasts can be, for example, human fibroblasts.

The methods can be performed wherein prior to incubating in the presence of the NDF, the stem cells are cultured in the presence of fibroblast growth factor 2 (FGF-2). The stem cells can also be cultured in the presence of conditioned media or chemically defined media such as mouse embryonic fibroblast-conditioned media (MEF-CM), mTeSR™ or StemPro™. The methods can be performed wherein prior to incubating in the presence of the NDF, at the same time as incubating in the presence of the NDF is initiated, or during incubating in the presence the NDF the use of FGF-2 is discontinued. The methods can be performed wherein the use of FGF-2 is discontinued at the same time as incubating in the presence of the NDF is initiated. The methods can be performed wherein the use of FGF-2 is discontinued at the same time as incubating in the presence of the NDF is initiated by replacing growth medium containing FGF-2 and lacking NDF with growth medium lacking FGF-2 and containing the NDF. The methods can be performed wherein prior to incubating in the presence of the NDF, at least a portion of the stem cells are cultured to a density of 1×10⁴ cells per square centimeter or greater.

The methods can be performed further comprising culturing the neural cells. The neural cells can be, for example, cultured on a treated polymer substrate. The treated polymer substrate can be, for example, CELLBIND™, or substrate treated with Matrigel™, Geltrex™, or fibronectin. The neural cells can be, for example, cultured in serum free conditions. The serum free conditions can comprise N2 supplement and B27 supplement. The neural cells can be, for example, cultured in the presence of fibroblast growth factor 2 (FGF-2). The neural cells can be cultured in the presence of epidermal growth factor (EGF). The neural cells can be cryopreservable. The neural cells can be, for example, passaged with Accutase or collagenase IV. The stem cells can be, for example, human stem cells. The stem cells can be, for example, embryonic stem cells (ESC). The stem cells can be, for example, derived from embryonic or fetal tissue. The stem cells can be, for example, derived from postfetal tissue. The stem cells can be, for example, derived from adult tissue. The stem cells can be, for example, derived from adult tissue. The stem cells can be, for example, induced pluripotent stem cells (iPSC). The stem cells can be, for example, derived from a subject in need of neural cells.

The neural cells can be, for example, neural stem cells. The neural cells can comprise, for example, neural stem cells. The neural cells can form, for example, neural tube-like structures. The methods can be performed further comprising differentiating the neural cells into differentiated neural cells. The neural cells can be, for example, differentiated neural cells. The neural cells can comprise, for example, neurons, astrocytes, oligodendrocytes, or a combination. The neural cells can comprise, for example, pyramidal neurons, motor neurons, spinal ventral horn motor neurons, neurons of the ventral mesencephalon, interneurons, glial cells, radial glial cells, retinal pigment epithelium, oligodendrocytes, dopamine neurons, GABA neurons, glutamate neurons, catecholinergic neurons, serotoninergic neurons, cholinergic neurons, or a combination. The neural cells can comprise, for example, pyramidal neurons, such as cortical pyramidal neurons. The neural cells can comprise, for example, neurons of the ventral mesencephalon (substantia nigra). The neural cells can comprise, for example, interneurons. The neural cells can comprise, for example, dopamine neurons, neurons that express dopamine, neurons that express molecules required for dopamine synthesis, neurons that express molecules resulting from dopamine metabolism, or a combination. The neural cells can comprise, for example, motor neurons. The neural cells can comprise, for example, spinal ventral horn motor neurons. The neural cells can comprise, for example, glial cells, including, for example, radial glial cells expressing brain lipid-binding protein (BLBP). The neural cells can comprise, for example, retinal pigment epithelium expressing, for example, the transcription factors OTX2, microphthalmia-associated transcription factor (MITE), and the tight-junction protein ZO-1. The neural cells can comprise, for example, oligodendrocytes expressing, for example, the surface marker RIP (2′,3′-cyclic nucleotide 3′-phosphodiesterase (CNPase)). The neural cells can comprise, for example, GABA neurons, neurons that express GABA, neurons that express molecules required for GABA synthesis, neurons that express molecules resulting from GABA metabolism, or a combination. The neural cells can comprise, for example, glutamate neurons, neurons that express glutamate, neurons that express molecules required for glutamate synthesis, neurons that express molecules resulting from glutamate metabolism, or a combination.

The neural cells can comprise, for example, catecholaminergic neurons, neurons that express catecholaminergically active molecules (molecules capable of activating catecholamine receptors; for example, L-DOPA, dopamine, norepinephrine, epinephrine), neurons that express molecules required for synthesis of catecholaminergically active molecules, neurons that express molecules resulting from metabolism of catecholaminergically active molecules, or a combination. The neural cells can comprise, for example, serotoninergic neurons, neurons that express serotoninergically active molecules (molecules capable of activating serotonin receptors), neurons that express molecules required for synthesis of serotoninergically active molecules, neurons that express molecules resulting from metabolism of serotoninergically active molecules, or a combination. The neural cells can comprise, for example, cholinergic neurons, neurons that express cholinergically active molecules (molecules capable of activating acetyl-choline receptors), neurons that express molecules required for synthesis of cholinergically active molecules, neurons that express molecules resulting from metabolism of cholinergically active molecules, or a combination.

The differentiated neural cells can comprise, for example, neurons, astrocytes, oligodendrocytes, or a combination. The differentiated neural cells can comprise, for example, pyramidal neurons, motor neurons, spinal ventral horn motor neurons, neurons of the ventral mesencephalon, interneurons, glial cells, radial glial cells, retinal pigment epithelium, oligodendrocytes, dopamine neurons, GABA neurons, glutamate neurons, catecholinergic neurons, serotoninergic neurons, cholinergic neurons, or a combination. The differentiated neural cells can comprise, for example, pyramidal neurons, such as cortical pyramidal neurons. The differentiated neural cells can comprise, for example, neurons of the ventral mesencephalon (substantia nigra). The differentiated neural cells can comprise, for example, interneurons. The differentiated neural cells can comprise, for example, dopamine neurons, neurons that express dopamine, neurons that express molecules required for dopamine synthesis, neurons that express molecules resulting from dopamine metabolism, or a combination. The differentiated neural cells can comprise, for example, motor neurons. The differentiated neural cells can comprise, for example, spinal ventral horn motor neurons. The differentiated neural cells can comprise, for example, glial cells, including, for example, radial glial cells expressing BLBP. The neural cells can comprise, for example, retinal pigment epithelium expressing, for example, OTX2, MITF, and ZO-1. The neural cells can comprise, for example, oligodendrocytes expressing, for example, the surface marker RIP. The differentiated neural cells can comprise, for example, GABA neurons, neurons that express GABA, neurons that express molecules required for GABA synthesis, neurons that express molecules resulting from GABA metabolism, or a combination. The differentiated neural cells can comprise, for example, glutamate neurons, neurons that express glutamate, neurons that express molecules required for glutamate synthesis, neurons that express molecules resulting from glutamate metabolism, or a combination.

The differentiated neural cells can comprise, for example, catecholaminergic neurons, neurons that express catecholaminergically active molecules, neurons that express molecules required for synthesis of catecholaminergically active molecules, neurons that express molecules resulting from metabolism of catecholaminergically active molecules, or a combination. The differentiated neural cells can comprise, for example, serotoninergic neurons, neurons that express serotoninergically active molecules, neurons that express molecules required for synthesis of serotoninergically active molecules, neurons that express molecules resulting from metabolism of serotoninergically active molecules, or a combination. The differentiated neural cells can comprise, for example, cholinergic neurons, neurons that express cholinergically active molecules, neurons that express molecules required for synthesis of cholinergically active molecules, neurons that express molecules resulting from metabolism of cholinergically active molecules, or a combination.

### BRIEF DESCRIPTION OF THE DRAWINGS

FIG. 1 shows MK expression during differentiation of human pluripotent stem cells and its neural-inducing activity. a, Increased MK immunoreactivity in areas of spontaneously differentiating hESC colonies. b, MK expression in various cell types as studied with Western blotting. c, d, Generation of neural tube-like structures in the presence of recombinant MK (see also FIG. 9). Note that these structures are morphologically different than previously reported “neural rosettes”. e, f, Removal of FGF-2 and application of recombinant MK generates PAX6-expressing cells under feeder-free conditions. Scale bars, 25 μm (d), 50 μm (c,e), 100 μm (a).

FIG. 2 shows directed neural induction of hESCs and hiPSCs. a, Overview of the feeder-free neural induction strategy. To minimize stress-induced effects that are inherent to the weekly cell passaging routine and which may interfere with controlled differentiation, neural induction was initiated on day 3. b, The vast majority of H9 hESCs differentiate into PAX6⁺/Nestin⁺ NPCs after 6 days in the presence of MK plus “DAP” (the acronym for the combination of small molecule inhibitors Dorsomorphin, A 83-01, and PNU-74654). c, Overview of a representative 10 cm cell culture dish with H9 hESCs after neural induction (day 6) showing that virtually all cells are PAX6+. d, Quantitative FACS analysis shows that 97% of total neuralized hESCs co-express PAX6 and Nestin (day 6). (Although H9 hESCs are illustrated, findings using the other hESC lines are similar.) (As shown in FIG. 11, the remaining 3% of cells that were not yet PAX6-positive were nevertheless OTX2-expressing, representing transitional cells en route and committed to a neuroectodermal/NPC lineage.) e, Direct comparison of EBs (day 6) and NPC cultures (day 6) for marker protein expression using immunoblotting. Note the absence of markers of non-neural lineages in NPC cultures but abundant in the EBs. f, Reprogrammed skin fibroblasts of a patient with spinal muscular atrophy are responsive to the neural induction protocol and generate highly pure populations of NPCs co-labeled for OTX2 and PAX6 (see also FIG. 10). Scale bars, 100 μm.

FIG. 3 shows differentiation potential of NPCs (derived from hESCs and hiPSCs) generated with the 6-day-neural-induction paradigm. a, b, NPCs derived from H9 cells after the first passage (day 8) expressing Nestin and OTX2. c-1, Representative examples showing neural progeny of reprogrammed human fetal lung fibroblasts (hiPSC 44.1). Cell type-specific markers demonstrate differentiation towards various more specialized neuronal (c,d, f-l) and glial (e) cells, including presumptive GABAergic neurons (d), motor neuron progenitors and motor neurons (f, g), and dopaminergic cells (h). i-1, Representative images showing neurons with the typical cell culture profile of young pyramidal-like neurons with large phase-bright cell bodies, large nuclei, and prominent nucleoli (arrows in i) co-expressing Tau protein and vGlut1 (j). Arrows in (i) and (j) are indicating the same neurons. (k) Note the dramatic difference in cell body size between GABAergic interneurons and vGlut1⁺ pyramidal-like neurons. At 1 month after neural induction, a punctate synaptophysin (Syn) immunostaining is decorating the neurites of these young pyramidal neurons. Neurons shown in i-l were derived from hiPSCs (line 34.1). See also FIG. 12. Scale bars, 100 μm (a, c), 50 μm (b, d-i).

FIG. 4 shows parallel signaling and orchestrated balance of the PI3K and MAPK pathways is necessary for neural induction. a, Western blot analysis comparing phosphorylation levels of AKT at Serine 473 and phospho-ERK1/2 in the presence of MK, IGF-1, and FGF-2. Note that both MK and IGF1 promote a similar degree of ERK1/2 phosphorylation while FGF-2 produces a significantly stronger activation. b, Chemical inhibition of the PI3K and MAPK signaling with low concentrations of LY294002 (10 μM) or U0126 (10 μM) does not impair neural induction. Under these conditions ˜95% of total cells still differentiate into NPCs co-expressing PAX6 and Nestin in response to MK+DAP as analyzed with FACS (compare with FIG. 2d). c, Dose-dependent disruption of neuralization with 40 μM LY294002 or 40 μM U0126 even in the presence of MK+DAP. Low concentrations of Rapamycin (20-40 nM) also block neural induction. All experiments involving chemical inhibitors were performed in parallel with H9 ESCs at the same passage. Importantly, when using the three inhibitors at the concentrations indicated, H9 ESCs continued to grow in all treatment groups. Typically, dishes were 40-50% confluent at the beginning of the experiments and reached complete confluency at day 6. Note also the numerous mitotic figures, some indicated by arrows. d, FACS analysis of PAX6-expressing NPCs confirms that blockade of PI3K or MAPK disrupts neural induction. e, Representative images showing neural differentiation in the presence of MK+DAP, FGF-2+DAP, and MK+FGF-2+DAP. Note the clear difference in the number of PAX6-expressing cells. FGF-2, despite its stronger activation of p-ERK 1/2 (Panel a) fails to promote large populations of Pax6+ cells. Note also that the addition of FGF-2 neutralizes the MK effect.

FIG. 5 shows proposed model for neural induction of pluripotent stem cells. Simultaneously active PI3K and MAPK pathways with concomitant blockade of TGF and canonical WNT signaling are sufficient and necessary to control neural lineage entry of hESCs and hiPSCs and to generate NPCs at high purity within 6 days. Neural differentiation is disrupted when PI3K, mTOR, or MEK1/2 are blocked by a single specific inhibitor (LY294002, Rapamycin, or U0126). Note that, apart from blocking TGFβ receptors, Dorsomorphin and A83-01 have indirect synergistic effects on the PI3K pathway by disinhibiting mTOR and p70S6K, respectively. For practical reasons this model does not show more of the reciprocal cross-talk and feedback loops between the various pathways, particularly between PI3K and MAPK. Since excess MAPK signaling with FGF-2 can antagonize neuralization as effectively as can insufficient signaling (see also FIG. 4e), components of the MAPK pathway are illustrated in smaller letters (compared to PI3K pathway members) to emphasize the fact that a modulated level of MAPK activity is required to preserve equilibrated PI3K-MAPK-mediated highly efficient neural induction.

FIG. 6 shows immunocytochemical analysis of MK expression in hESCs (lines H9, H1, HUES13) and hiPSCs (HS27-iPS). HS27 is the parent human foreskin fibroblast cell line of HS27-iPS and serves as a negative control; note that, appropriately, neither MK nor OCT4 is expressed. Scale bar, 100 μm.

FIG. 7 shows midkine receptors PTPz and ALK are expressed by hESCs and hiPSCs. Representative images display punctate staining pattern for both receptors. SMA, spinal muscular atrophy. Scale bars, 50 or 100 μm.

FIG. 8 shows generation and characterization of hiPSCs derived from human fibroblasts. a, Time-line for retroviral reprogramming b, Successfully reprogrammed hiPSCs are morphologically indistinguishable from hESCs. c, Alkaline Phosphatase staining at different time points shows that positive colonies are detectable as early as day 13. d, The surface marker TRA1-81 is only expressed by distinct OCT4-expressing colonies (arrowhead) representing a reliable marker for reprogrammed cells as suggested (Lowry et al. PNAS 105:2883-2888, 2008) but not by tumor cells (arrows). e, Nanog expression by hiPSCs. f-g, Embryoid body differentiation of HS27-iPSCs showing in vitro pluripotency and generation of ectodermal (neural cell adhesion molecule, NCAM) (f), endodermal (alpha-feto-protein, AFP) (g), and mesodermal (Brachyury) (h) cells. Scale bars, 100 μm (d, d′), 50 μm (e-h).

FIG. 9 shows examples of neural tube-like (NTL) structures generated by MK-induced differentiation. a, Free-floating individual NTLs after being detached from human feeders. b, After attaching to the laminin substrate, neuron-like cells migrate out from the NTL and spontaneously develop long neurites.

FIG. 10 shows robust neural differentiation is reproducible with various hESCs and hiPSCs when cultures are treated according to FIG. 2a. Representative images show vast majority of cells co-expressing early neural progenitor cell markers OTX2 and PAX6. See FIG. 2d of main text for FACS-based quantification (−97% are PAX6⁺; all are OTX2⁺, including the 3% which are not yet PAX6⁺. See also the absence of OCT4 expression at day 6 after neural induction. Scale bars, 50 μm (a,c), 100 μm (b).

FIG. 11 shows mapping the transition from pluripotent stem cells to early neuroectodermal cells. a, Overview of marker protein expression during neural induction. b, Expression of OTX2 is detectable as early as 2 days after initiation of neural induction. Despite variable immunoreactivities reflecting dynamic expression changes, OTX2 and OCT4 are still widely co-localized at this time point. OTX2, in other words, represents a transitional marker in the early phases of neural induction. See also the absence of PAX6 expression at day 2 of the neuralization process. c, By day 6, only a few cells are detectable that are slowly losing and weakly expressing OCT4; of those all express OTX2 at variable levels, indicating transitional cells en route to neural commitment (encircled cells). None of those cells yield non-neural lineages. Scale bars, 50 μm (b), 25 μm (c).

FIG. 12 shows representative examples of neuronal cultures with the typical in vitro appearance of young cortical pyramidal neurons. a, Overview phase contrast images of neuronal cultures taken at the same magnification. Note the difference in cell body size and the purity of the cultures. Arrows indicate cells with large phase-bright somata, large nuclei, and prominent nucleoli extending numerous processes. b, c, The cells indicated in (a) typically co-express the neuronal markers Tau, vGlut1, and neurogranin (NG). Again, these neurons display prominent nucleoli, now visualized with phase contrast or differential interference contrast (DIC) microscopy to highlight the nucleoli and long neurites. All examples derived from hiPSCs (34.1). Scale bars, 50 μm.

FIG. 13 shows chemical inhibition of the PI3K and MAPK pathways promotes endodermal differentiation even when exposed to MK+DAP. Under the conditions indicated, a fraction of cells express the endodermal marker SOX17 or PAX6, respectively. Scale bar, 25 μm.

FIG. 14 shows IGF-1 can replace recombinant MK during neural induction. Highly efficient production of PAX6-expressing cells in 6 days when IGF-1 (100 ng/mL) is applied together with DAP. In contrast, application of FGF-2+DAP or MK+FGF-2+DAP generates only a few Pax6⁺ cells (see also FIG. 4e).

### DESCRIPTION

The disclosed method and compositions may be understood more readily by reference to the following detailed description of particular embodiments and the Example included therein and to the Figures and their previous and following description.

Prior to the present discoveries, directed and complete neuralization of human pluripotent stem cells in a time frame mirroring embryogenesis has not been achieved because the fundamental mechanisms underlying neural induction remain elusive. As described herein, those critical signaling pathways have been elucidated and demonstrate that, by their manipulation, human embryonic stem cells (hESCs) and human induced pluripotent stem cells (hiPSCs) can be directly and completely converted into neuroectoderm in feeder-free monolayer cultures. It has been discovered and demonstrated herein that undifferentiated hESCs and hiPSCs constitutively express the growth and differentiation factor midkine (MK). Autocrine MK is strongly upregulated during spontaneous and embryoid body (EB) differentiation and recombinant MK exhibits profound neural-inducing activity. MK promotes a critical balance between high PI3K and moderate MAPK signaling. Insulin-like growth factor 1 (IGF-1) creates a similar balance. Direct and exclusive neural lineage entry of hESC and hiPSCs is induced in the presence of either MK or IGF-1 when the TGFβ superfamily and canonical WNT (mediators of pluripotency and non-neural differentiation) are concomitantly blocked with three small molecules: Dorsomorphin, A83-01, and PNU-74654. This highly efficient inductive process can be disrupted by a single chemical inhibitor of the PI3K/MAPK pathways (LY294002, Rapamycin, or U0126). Similarly, overstimulation of MAPK (e.g., through the use of FGF-2) can also disrupt neuralization. However, when allowed to proceed, balanced PI3K-MAPK signaling in parallel with TGFβ superfamily and Wnt blockade insures the production of virtually pure neural precursor cell (NPC) cultures in only 6 days (97% PAX6⁺/Nestin⁺; with the remaining 3% transitional cells en route to committed NPCs). Further differentiation can then generate an array of more specialized neuronal and glial phenotypes (e.g., tyrosine hydroxylase+neurons, Olig2+/HB9+ neurons, large glutamatergic pyramidal-like neurons). The paradigm described here, which indicates an instructive rather than a neural default mechanism, allows the rapid and systematic production of unlimited numbers of uniform, defined neural phenotypes for developmental studies, drug discovery, and regenerative medicine.

It has been discovered that inducing or creating balanced signaling in the phosphatidylinositol 3-kinase (PI3K) and mitogen-activated protein kinase (MAPK) signaling pathway, inhibition of the transforming growth factor-β (TGF-β) superfamily signaling pathway, and inhibition of the Wnt signaling pathway in pluripotent stem cells robustly produces neural stem cells. This inductive process is highly efficient, producing a high percentage of neural stem cells expressing Pax-6 and Nestin. As an example, excellent induction of neural stem cells was accomplished by exposing pluripotent stem cells to factors that activate both the PI3K and MAPK signaling pathways and factors that inhibit the TGF-β superfamily and the Wnt signaling pathways. Induction to neural stem cells was efficient when blockade of the TGF-β superfamily and the Wnt signaling pathways effected inhibition of non-ectodermal development. The discovered induction does not require feeder layer cells with the result that the resulting neural stem cell cultures can be essentially consist of neural stem cells. The neural stem cells produced by the disclosed methods can be used to produce any desired neural cell type via appropriate differentiation.

Disclosed are compositions and methods for producing neural cells from stem cells. For example, disclosed are methods involving generation of neural stem cells from pluripotent stem cells. Also disclosed are neural cells and neural stem cells produced in the disclosed methods. Also disclosed are compositions and methods of using neural cells and neural stem cells produced in the disclosed methods. Also disclosed are compositions and methods of treating a subject using the neural cells and neural stem cells produced in the disclosed methods. Also disclosed are compositions and methods of detecting a state or characteristic of neural cells and neural stem cells produced in the disclosed methods. Also disclosed are compositions and methods testing conditions for differentiation of neural stem cells using the neural cells and neural stem cells produced in the disclosed methods. Also disclosed are methods of producing differentiated neural cells using the neural cells and neural stem cells produced in the disclosed methods.

The first human embryonic stem cell (hESC) lines were established in 1998 by James Thomson and colleagues. However, even after more than a decade of work with hESCs the practical use of these cells has been very limited because of our poor knowledge of the molecular mechanisms that control their differentiation. Specifically, the earliest differentiation step from a pluripotent hESC to a neuroectodermal cell type, also called neural induction, remains poorly understood. So far, the production of neural cells from hESCs in solely based on empirical methods such as embryoid body formation, co-culture with murine stromal cells, and overgrowth of hESC cultures in order to obtain spontaneously differentiating neural cells.

The development of an efficient neural induction protocol that allows the controlled and directed production of unlimited numbers of human neural stem cell would be useful for basic research and future clinical cell therapy based on neural stem cells. The availability of an efficient neural induction protocol would allow systematic and rational methods to generate the three major cell types of the central nervous system (i.e. various neuronal cell types such as cortical pyramidal neurons, dopamine neurons and motorneurons, astrocytes, and oligodendrocytes).

It has been discovered that providing the appropriate cell culture conditions and modulation (for example, activation, inhibition) of specific signaling pathways mediates differentiation of pluripotent stem cells (PSCs). Based on this, protocols were developed for neural induction and production of neural cells from pluripotent stem cells, including, for example, embryonic stem cells and reprogrammed somatic cells with acquired pluripotency.

In spontaneously differentiating colonies of stem cells, such as hESCs, the earliest neural stem cell (identified by, for example, expressing the transcription factor Pax-6, Otx2, Nestin, or a combination) emerges preferentially in regions with high cell densities. Stem cells can be incubated on, for example, fibroblasts (for example, hESCs can be grown on human fibroblasts). This can allow the stem cells to grow to a higher density and can result in the stem cells growing and differentiating as densely packed cell colonies. Culturing stem cells on non-homologous fibroblasts (for example, culturing hESCs on mouse fibroblasts) or under feeder-free conditions (for example, Matrigel™) generally does not result such a compacted growth of the stem cells and reduces their neural differentiation potential. In the absence of pluripotency maintaining factors (such as, for example, fibroblast growth factor 2 (FGF-2)) stem cells tend to flatten and spread out generating cobblestone-like cells which do not express, for example, Pax-6, Otx2, Nestin, or a combination.

In the presence of FGF-2, for example, stem cells placed on fibroblasts can be grown for extended periods of time in an undifferentiated pluripotent state. Hence, growing stem cells simply on fibroblasts does not allow and is not sufficient for neural differentiation. It has been discovered that the removal of FGF-2 and administration of specific differentiation factors allows neural differentiation. In particular, it has been discovered that activating the phosphatidylinositol 3-kinase signaling pathway allows neural differentiation. Activation of the phosphatidylinositol 3-kinase signaling pathway can also be combined with activation of the MAPK signaling pathway. Activation of the phosphatidylinositol 3-kinase signaling pathway and of the MAPK signaling pathway can be balanced. Activation of the phosphatidylinositol 3-kinase signaling pathway can also be combined with inhibition of the TGF-β superfamily signaling pathway, inhibition of the Wnt signaling pathway, or a combination. Activation of the phosphatidylinositol 3-kinase signaling pathway can also be combined with activation of the MAPK signaling pathway, inhibition of the TGF-β superfamily signaling pathway, inhibition of the Wnt signaling pathway, or a combination. Each of these pathway modulations can be used alone or in any combination. The combination of these pathway modulations is particularly useful.

Activation of the phosphatidylinositol 3-kinase signaling pathway can also be combined with activation of the Notch signaling pathway, inhibition of the TGF-β superfamily signaling pathway, inhibition of the Wnt signaling pathway, or a combination. Each of these pathway modulations can be used alone or in any combination. The combination of these pathway modulations is particularly useful. Activation of the phosphatidylinositol 3-kinase signaling pathway can also be combined with inhibition of the TGF-β superfamily signaling pathway, inhibition of the Wnt signaling pathway, activation of the protein kinase A signaling pathway, or a combination. Activation of the phosphatidylinositol 3-kinase signaling pathway can also be combined with activation of the MAPK signaling pathway, inhibition of the TGF-β superfamily signaling pathway, inhibition of the Wnt signaling pathway, activation of the protein kinase A signaling pathway, or a combination. Each of these pathway modulations can be used alone or in any combination. The combination of these pathway modulations is particularly useful. Activation of the phosphatidylinositol 3-kinase signaling pathway can also be combined with activation of the Notch signaling pathway, inhibition of the TGF-β superfamily signaling pathway, inhibition of the Wnt signaling pathway, activation of the protein kinase A signaling pathway or a combination. Activation of the phosphatidylinositol 3-kinase signaling pathway can also be combined with activation of the MAPK signaling pathway, activation of the Notch signaling pathway, inhibition of the TGF-β superfamily signaling pathway, inhibition of the Wnt signaling pathway, activation of the protein kinase A signaling pathway or a combination. Each of these pathway modulations can be used alone or in any combination. The combination of these pathway modulations is particularly useful.

It was discovered, for example, that Midkine aids and allows neural differentiation. It was demonstrated that Midkine is an autocrine factor that undifferentiated pluripotent hESCs express at low levels but strongly up-regulate during spontaneous differentiation and embryoid body formation. Removal of FGF-2 and application of 40-100 ng/mL Midkine to hESCs on human fibroblasts was discovered to generate neural stem cells expressing Pax-6, Otx2, Nestin, or a combination and neural tube-like structures after 6-8 days. In the presence of Midkine, these neural tube-like structures (also expressing Pax-6, Otx2, Nestin, or a combination) emerge directly from individual hESC colonies.

The efficiency of neural induction and the number of cells and hESC colonies expressing Pax-6, Otx2, Nestin, or a combination can be increased when Midkine application (and/or other activators of the phosphatidylinositol 3-kinase signaling pathway) is combined with inhibition of the TGF-β superfamily signaling pathway. The efficiency of neural induction and the number of cells and hESC colonies expressing Pax-6, Otx2, Nestin, or a combination can be increased when Midkine application (and/or other activators of the phosphatidylinositol 3-kinase signaling pathway) is combined with inhibition of the Wnt signaling pathway. The efficiency of neural induction and the number of cells and hESC colonies expressing Pax-6, Otx2, Nestin, or a combination can be increased when Midkine application (and/or other activators of the phosphatidylinositol 3-kinase signaling pathway) is combined with inhibition of the TGF-β superfamily signaling pathway, inhibition of the Wnt signaling pathway, or a combination. Thus, for example, neural induction can be stimulated by activating the phosphatidylinositol 3-kinase signaling pathway, inhibition of the TOE-β superfamily signaling pathway, or a combination. As another example, neural induction can be stimulated by activating the phosphatidylinositol 3-kinase signaling pathway in combination with inhibition of the TOE-β superfamily signaling pathway. As another example, neural induction can be stimulated by activating the phosphatidylinositol 3-kinase signaling pathway, inhibition of the Wnt signaling pathway, or a combination. As another example, neural induction can be stimulated by activating the phosphatidylinositol 3-kinase signaling pathway in combination with inhibition of the Wnt signaling pathway. As another example, neural induction can be stimulated by activating the phosphatidylinositol 3-kinase signaling pathway, inhibition of the TGF-β superfamily signaling pathway, inhibition of the Wnt signaling pathway, or a combination. As another example, neural induction can be stimulated by activating the phosphatidylinositol 3-kinase signaling pathway in combination with inhibition of the TGF-β superfamily signaling pathway, inhibition of the Wnt signaling pathway, or a combination. As another example, neural induction can be stimulated by activating the phosphatidylinositol 3-kinase signaling pathway in combination with activation of the MAPK signaling pathway, inhibition of the TGF-β superfamily signaling pathway, inhibition of the Wnt signaling pathway, or a combination.

The efficiency of neural induction and the number of cells and hESC colonies expressing Pax-6, Otx2, Nestin, or a combination can also be increased when Midkine application (and/or other activators of the phosphatidylinositol 3-kinase signaling pathway) is combined with activation of the Notch signaling pathway. This can be accomplished by, for example, using Delta-1, Delta-2, Delta-3, Delta-4, Jagged-1, Jagged-2, or a combination. The efficiency of neural induction and the number of cells and hESC colonies expressing Pax-6, Otx2, Nestin, or a combination can also be increased when Midkine application (and/or other activators of the phosphatidylinositol 3-kinase signaling pathway) is combined with activation of the protein kinase A signaling pathway. This can be accomplished by, for example, using the drug Forskolin. Forskolin is a widely used to increase the intracellular level of cyclic AMP (cAMP) by activating the enzyme adenylyl cyclase. Thus, for example, neural induction can be stimulated by activating the phosphatidylinositol 3-kinase signaling pathway, activation of the Notch signaling pathway, or a combination. As another example, neural induction can be stimulated by activating the phosphatidylinositol 3-kinase signaling pathway in combination with activation of the Notch signaling pathway. As another example, neural induction can be stimulated by activating the phosphatidylinositol 3-kinase signaling pathway, activation of the protein kinase A signaling pathway or a combination. As another example, neural induction can be stimulated by activating the phosphatidylinositol 3-kinase signaling pathway in combination with activation of the protein kinase A signaling pathway or a combination. As another example, neural induction can be stimulated by activating the phosphatidylinositol 3-kinase signaling pathway, activation of the Notch signaling pathway, activation of the protein kinase A signaling pathway or a combination. As another example, neural induction can be stimulated by activating the phosphatidylinositol 3-kinase signaling pathway in combination with activation of the Notch signaling pathway, activation of the protein kinase A signaling pathway or a combination.

The efficiency of neural induction and the number of cells and hESC colonies expressing Pax-6, Otx2, Nestin, or a combination can be increased when activation of the phosphatidylinositol 3-kinase signaling pathway, inhibition of the TGF-β superfamily signaling pathway, and inhibition of the Wnt signaling pathway, or a combination, is combined with activation of the Notch signaling pathway. This can be accomplished by, for example, using Delta-1, Delta-2, Delta-3, Delta-4, Jagged-1, Jagged-2, or a combination. The efficiency of neural induction and the number of cells and hESC colonies expressing Pax-6, Otx2, Nestin, or a combination can be increased when activation of the phosphatidylinositol 3-kinase signaling pathway, inhibition of the TGF-β superfamily signaling pathway, and inhibition of the Wnt signaling pathway, or a combination, is combined with activation of the protein kinase A signaling pathway. This can be accomplished by, for example, using the drug Forskolin. Thus, for example, neural induction can be stimulated by activating the phosphatidylinositol 3-kinase signaling pathway, activation of the Notch signaling pathway, inhibition of the TGF-β superfamily signaling pathway, inhibition of the Wnt signaling pathway, or a combination. As another example, neural induction can be stimulated by activating the phosphatidylinositol 3-kinase signaling pathway in combination with activation of the Notch signaling pathway, inhibition of the TGF-β superfamily signaling pathway, inhibition of the Wnt signaling pathway, or a combination. As another example, neural induction can be stimulated by activating the phosphatidylinositol 3-kinase signaling pathway and MAPK signaling pathway in combination with activation of the Notch signaling pathway, inhibition of the TOE-β superfamily signaling pathway, inhibition of the Wnt signaling pathway, or a combination. As another example, neural induction can be stimulated by activating the phosphatidylinositol 3-kinase signaling pathway, inhibition of the TGF-β superfamily signaling pathway, inhibition of the Wnt signaling pathway, activation of the protein kinase A signaling pathway or a combination. As another example, neural induction can be stimulated by activating the phosphatidylinositol 3-kinase signaling pathway in combination with inhibition of the TGF-β superfamily signaling pathway, inhibition of the Wnt signaling pathway, activation of the protein kinase A signaling pathway or a combination. As another example, neural induction can be stimulated by activating the phosphatidylinositol 3-kinase signaling pathway and MAPK signaling pathway in combination with inhibition of the TGF-β superfamily signaling pathway, inhibition of the Wnt signaling pathway, activation of the protein kinase. As another example, neural induction can be stimulated by activating the phosphatidylinositol 3-kinase signaling pathway, activation of the Notch signaling pathway, inhibition of the TGF-β superfamily signaling pathway, inhibition of the Wnt signaling pathway, activation of the protein kinase A signaling pathway or a combination. As another example, neural induction can be stimulated by activating the phosphatidylinositol 3-kinase signaling pathway in combination with activation of the Notch signaling pathway, inhibition of the TGF-β superfamily signaling pathway, inhibition of the Wnt signaling pathway, activation of the protein kinase A signaling pathway or a combination. As another example, neural induction can be stimulated by activating the phosphatidylinositol 3-kinase signaling pathway and MAPK signaling pathway in combination with activation of the Notch signaling pathway, inhibition of the TGF-β superfamily signaling pathway, inhibition of the Wnt signaling pathway, activation of the protein kinase A signaling pathway or a combination.

Disclosed are methods of producing neural cells, the method comprising, for example, incubating pluripotent stem cells in the presence of an amount of one or more Neural Development Factors (NDF) sufficient to generate cells expressing Pax-6, Otx2, Nestin, or a combination, thereby producing neural cells. Also disclosed are neural cells produced by one or more of the disclosed methods. Also disclosed are neural cells produced by the method comprising incubating pluripotent stem cells in the presence of an amount of one or more Neural Development Factors (NDF) sufficient to generate cells expressing Pax-6, Otx2, Nestin, or a combination.

Also disclosed are methods of treating a subject, the method comprising administering a neural cell produced by one or more of the disclosed methods. Also disclosed are methods of treating a subject, the method comprising incubating pluripotent stem cells in the presence of an amount of one or more Neural Development Factors (NDF) sufficient to generate cells expressing Pax-6, Otx2, Nestin, or a combination, thereby producing neural cells, and administering one or more of the neural cells to the subject. The stem cell can be, for example, from the same species as the subject. The stem cell can be, for example, from the subject.

Also disclosed are methods of detecting a state or characteristic of a cell, the method comprising detecting the state or characteristic in a neural cell produced by one or more of the disclosed methods. Also disclosed are methods of testing conditions for differentiation of neural stem cells, the method comprising exposing a neural cell produced by one or more of the disclosed methods to test conditions and determining if the neural stem cells differentiate into a cell type of interest. The cell type of interest can be, for example, neuron, astrocyte, oligodendrocyte, or a combination.

Also disclosed are methods of producing differentiated neural cells, the method comprising incubating pluripotent stem cells in the presence of an amount of one or more Neural Development Factors (NDF) sufficient to generate cells expressing Pax-6, Otx2, Nestin, or a combination, thereby producing neural cells, and differentiating the neural cells into differentiated neural cells. The neural cells can be differentiated into differentiated neural cells by, for example, incubating the neural cells under differentiation conditions. The differentiation conditions can be chosen based on the type of differentiated neural cells desired or sought.

Also disclosed are methods of producing neural cells, the method comprising incubating pluripotent stem cells in the presence of an amount of one or more Neural Development Factors (NDF) sufficient to generate cells expressing Pax-6, Otx2, Nestin, or a combination, thereby producing neural cells, wherein the NDF activate the phosphatidylinositol 3-kinase (PI3K) signaling pathway, inhibit transforming growth factor-β (TGF-β) superfamily signaling pathway, and inhibit the Wnt signaling pathway, wherein the NDF comprise Midkine, Pleiotrophin, insulin-like growth factor-1 (IGF-1), or a combination; A83-01, SB431542, or a combination; dorsopmorphin; and PNU-74654, Dickkopf, or a combination.

Also disclosed are methods of producing neural cells, the method comprising (a) culturing pluripotent stem cells in the presence of fibroblast growth factor 2 (FGF-2); (b) incubating the stem cells in the presence of an amount of one or more Neural Development Factors (NDF) sufficient to generate cells expressing Pax-6, Otx2, Nestin, or a combination, thereby producing neural cells, wherein the NDF activate the phosphatidylinositol 3-kinase signaling pathway, inhibit the TGF-β superfamily signaling pathway, and inhibit the Wnt signaling pathway, wherein the NDF comprise Midkine, Pleiotrophin, insulin-like growth factor-1, or a combination; A83-01, SB431542, or a combination; dorsopmorphin; and PNU-74654, Dickkopf, or a combination, wherein prior to incubating in the presence of the NDF, at the same time as incubating in the presence of the NDF is initiated, or during incubating in the presence the NDF the use of FGF-2 is discontinued; and (c) culturing the neural cells, wherein the neural cells are cultured on a treated polymer substrate, wherein the neural cells are cultured in serum free conditions, wherein the neural cells are cultured in the presence of fibroblast growth factor 2.

Also disclosed are methods of producing neural cells, the method comprising (a) culturing pluripotent stem cells in the presence of fibroblast growth factor 2; (b) incubating the stem cells in the presence of an amount of one or more Neural Development Factors (NDF) sufficient to generate cells expressing Pax-6, Otx2, Nestin, or a combination, thereby producing neural cells, wherein the NDF activate the phosphatidylinositol 3-kinase signaling pathway, activate the Notch signaling pathway, inhibit the TGF-β superfamily signaling pathway, and inhibit the Wnt signaling pathway, wherein the NDF comprise Midkine, Pleiotrophin, insulin-like growth factor-1, or a combination; Delta-1, Delta-2, Delta-3, Delta-4, Jagged-1, Jagged-2, or a combination; A83-01, SB431542, or a combination; dorsopmorphin; and PNU-74654, Dickkopf, or a combination, wherein prior to incubating in the presence of the NDF, at the same time as incubating in the presence of the NDF is initiated, or during incubating in the presence the NDF the use of FGF-2 is discontinued; and (c) culturing the neural cells, wherein the neural cells are cultured on a treated polymer substrate, wherein the neural cells are cultured in serum free conditions, wherein the neural cells are cultured in the presence of fibroblast growth factor 2.

The treated polymer substrate can be, for example, CELLBIND™, or substrate treated with Matrigel™, Geltrex™, or fibronectin. The treated polymer substrate can comprise, for example, CELLBIND™, or substrate treated with Matrigel™, Geltrex™, or fibronectin. The serum free conditions can comprise N2 supplement and B27 supplement. The neural cells can be cultured in the presence of epidermal growth factor (EGF). The neural cells can be cryopreservable. The stem can also be cultured in the presence of conditioned media or chemically defined media such as mouse embryonic fibroblast-conditioned media (MEF-CM), mTeSR™ or StemPro™. The stem cells can also be incubated in the presence of 20% knockout serum replacement (KSR).

The disclosed methods for producing neural cells from stem cells can involve incubation with NDF. The methods can further comprise pre-incubation of the stem cells prior to the incubation. The methods can further comprise culturing the neural cells produced by the incubation. The methods can further comprise a transition from pre-incubation and incubation. The methods can further comprise a transition from incubation and culturing. The methods can further provide further differentiation of the neural cells during or after culturing. For example, neural stem cells produced during incubation can be differentiated into neural cell lineages, tissue types and/or cell types during or after culturing.

The NDF can, for example, activate the phosphatidylinositol 3-kinase (PI3K) signaling pathway. The NDF can, for example, activate the mitogen-activated protein kinase (MAPK) signaling pathway. The NDF can, for example, activate the phosphatidylinositol 3-kinase signaling pathway and can activate the MAPK signaling pathway. The NDF can, for example, activate the phosphatidylinositol 3-kinase signaling pathway and the MAPK signaling pathway. The NDF can, for example, activate the phosphatidylinositol 3-kinase signaling pathway and the MAPK signaling pathway in a balanced manner. The NDF can, for example, inhibit the TGF-β superfamily signaling pathway. The NDF can, for example, inhibit the Wnt signaling pathway. The NDF can, for example, activate the phosphatidylinositol 3-kinase signaling pathway, inhibit the TGF-β superfamily signaling pathway, and inhibit the Wnt signaling pathway. The NDF can, for example, activate the phosphatidylinositol 3-kinase signaling pathway, inhibit the TGF-β superfamily signaling pathway, inhibit the Wnt signaling pathway, or any combination of these. The NDF can, for example, activate the phosphatidylinositol 3-kinase signaling pathway, activate the MAPK signaling pathway, inhibit the TGF-β superfamily signaling pathway, and inhibit the Wnt signaling pathway. The Wnt signaling pathway can be, for example, inhibited after the β-catenin destruction complex. The NDF can, for example, activate the Notch signaling pathway. The NDF can, for example, activate the phosphatidylinositol 3-kinase signaling pathway, activate the Notch signaling pathway, inhibit the TGF-β superfamily signaling pathway, and inhibit the Wnt signaling pathway. The NDF can, for example, activate the phosphatidylinositol 3-kinase signaling pathway, activate the Notch signaling pathway, inhibit the TGF-β superfamily signaling pathway, inhibit the Wnt signaling pathway, or any combination of these.

The NDF can comprise, for example, an activator of the phosphatidylinositol 3-kinase signaling pathway. The NDF can comprise, for example, an activator of the MAPK signaling pathway. The NDF can comprise, for example, an activator of the phosphatidylinositol 3-kinase signaling pathway and the MAPK signaling pathway. The NDF can comprise, for example, Midkine, Pleiotrophin, insulin-like growth factor-1, or a combination. The NDF can comprise, for example, Midkine, insulin-like growth factor-1, or a combination. The NDF can comprise, for example, Midkine. The NDF can comprise, for example, an activator of the phosphatidylinositol 3-kinase signaling pathway and an activator of the MAPK signaling pathway. The NDF can comprise, for example, an inhibitor of the TGF-β superfamily signaling pathway. The NDF can comprise, for example, A83-01, SB431542, or a combination. The NDF can comprise, for example, dorsomorphin. The NDF can comprise, for example, dorsomorphin and A83-01. The NDF can comprise, for example, an inhibitor of the Wnt signaling pathway. The NDF can comprise, for example, PNU-74654, Dickkopf, or a combination. The NDF can comprise, for example, an activator of the phosphatidylinositol 3-kinase signaling pathway, an inhibitor of the TGF-β superfamily signaling pathway, and an inhibitor of the Wnt signaling pathway. The NDF can comprise, for example, an activator of the phosphatidylinositol 3-kinase signaling pathway, an inhibitor of the TGF-β superfamily signaling pathway, an inhibitor of the Wnt signaling pathway, or any combination of these. The NDF can comprise, for example, Midkine, Pleiotrophin, insulin-like growth factor-1, or a combination; A83-01, SB431542, or a combination; dorsopmorphin; and PNU-74654, Dickkopf, or a combination. The NDF can comprise, for example, Midkine, Pleiotrophin, insulin-like growth factor-1, or a combination; A83-01, SB431542, or a combination; dorsopmorphin; PNU-74654, Dickkopf, or a combination; or any combination of these. The NDF can comprise, for example, Midkine, A83-01, dorsopmorphin, and PNU-74654. The NDF can comprise, for example, insulin-like growth factor-1, A83-01, dorsopmorphin, and PNU-74654.

The NDF can comprise, for example, an activator of the Notch signaling pathway. The NDF can comprise, for example, Delta-1, Delta-2, Delta-3, Delta-4, Jagged-1, Jagged-2, or a combination. The NDF can comprise, for example, an activator of the phosphatidylinositol 3-kinase signaling pathway, an activator of the Notch signaling pathway, an inhibitor of the TGF-β superfamily signaling pathway, and an inhibitor of the Wnt signaling pathway. The NDF can comprise, for example, an activator of the phosphatidylinositol 3-kinase signaling pathway, an activator of the Notch signaling pathway, an inhibitor of the TGF-β superfamily signaling pathway, an inhibitor of the Wnt signaling pathway, or any combination of these. The NDF can comprise, for example, Midkine, Pleiotrophin, insulin-like growth factor-1, or a combination; Delta-1, Delta-2, Delta-3, Delta-4, Jagged-1, Jagged-2, or a combination; A83-01, SB431542, or a combination; dorsopmorphin; and PNU-74654, Dickkopf, or a combination. The NDF can comprise, for example, Midkine, Pleiotrophin, insulin-like growth factor-1, or a combination; Delta-1, Delta-2, Delta-3, Delta-4, Jagged-1, Jagged-2, or a combination; A83-01, SB431542, or a combination; dorsopmorphin; PNU-74654, Dickkopf, or a combination; or any combination of these.

The NDF can consist of, for example, an activator of the phosphatidylinositol 3-kinase signaling pathway. The NDF can consist of, for example, an activator of the MAPK signaling pathway. The NDF can consist of, for example, an activator of the phosphatidylinositol 3-kinase signaling pathway and the MAPK signaling pathway. The NDF can consist of, for example, Midkine, Pleiotrophin, insulin-like growth factor-1, or a combination. The NDF can consist of, for example, Midkine, insulin-like growth factor-1, or a combination. The NDF can consist of, for example, Midkine. The NDF can consist of, for example, an activator of the phosphatidylinositol 3-kinase signaling pathway and an activator of the MAPK signaling pathway. The NDF can consist of, for example, an inhibitor of the TGF-β superfamily signaling pathway. The NDF can consist of, for example, A83-01, SB431542, or a combination. The NDF can consist of, for example, dorsomorphin. The NDF can consist of, for example, dorsomorphin and A83-01. The NDF can consist of, for example, an inhibitor of the Wnt signaling pathway. The NDF can consist of, for example, PNU-74654, Dickkopf, or a combination. The NDF can consist of, for example, an activator of the phosphatidylinositol 3-kinase signaling pathway, an inhibitor of the TGF-β superfamily signaling pathway, and an inhibitor of the Wnt signaling pathway. The NDF can consist of, for example, an activator of the phosphatidylinositol 3-kinase signaling pathway, an inhibitor of the TGF-β superfamily signaling pathway, an inhibitor of the Wnt signaling pathway, or any combination of these. The NDF can consist of, for example, Midkine, Pleiotrophin, insulin-like growth factor-1, or a combination; A83-01, SB431542, or a combination; dorsopmorphin; and PNU-74654, Dickkopf, or a combination. The NDF can consist of, for example, Midkine, Pleiotrophin, insulin-like growth factor-1, or a combination; A83-01, SB431542, or a combination; dorsopmorphin; PNU-74654, Dickkopf, or a combination; or any combination of these. The NDF can consist of, for example, Midkine, A83-01, dorsopmorphin, and PNU-74654. The NDF can consist of, for example, insulin-like growth factor-1, A83-01, dorsopmorphin, and PNU-74654.

The NDF can consist of, for example, an activator of the Notch signaling pathway. The NDF can consist of, for example, Delta-1, Delta-2, Delta-3, Delta-4, Jagged-1, Jagged-2, or a combination. The NDF can consist of, for example, an activator of the phosphatidylinositol 3-kinase signaling pathway, an activator of the Notch signaling pathway, an inhibitor of the TGF-β superfamily signaling pathway, and an inhibitor of the Wnt signaling pathway. The NDF can consist of, for example, an activator of the phosphatidylinositol 3-kinase signaling pathway, an activator of the Notch signaling pathway, an inhibitor of the TGF-β superfamily signaling pathway, an inhibitor of the Wnt signaling pathway, or any combination of these. The NDF can consist of, for example, Midkine, Pleiotrophin, insulin-like growth factor-1, or a combination; Delta-1, Delta-2, Delta-3, Delta-4, Jagged-1, Jagged-2, or a combination; A83-01, SB431542, or a combination; dorsopmorphin; and PNU-74654, Dickkopf, or a combination. The NDF can consist of, for example, Midkine, Pleiotrophin, insulin-like growth factor-1, or a combination; Delta-1, Delta-2, Delta-3, Delta-4, Jagged-1, Jagged-2, or a combination; A83-01, SB431542, or a combination; dorsopmorphin; PNU-74654, Dickkopf, or a combination; or any combination of these.

The NDF can consist essentially of, for example, an activator of the phosphatidylinositol 3-kinase signaling pathway. The NDF can consist essentially of, for example, an activator of the MAPK signaling pathway. The NDF can consist essentially of, for example, an activator of the phosphatidylinositol 3-kinase signaling pathway and the MAPK signaling pathway. The NDF can consist essentially of, for example, Midkine, Pleiotrophin, insulin-like growth factor-1, or a combination. The NDF can consist essentially of, for example, Midkine, insulin-like growth factor-1, or a combination. The NDF can consist essentially of, for example, Midkine. The NDF can consist essentially of, for example, an activator of the phosphatidylinositol 3-kinase signaling pathway and an activator of the MAPK signaling pathway. The NDF can consist essentially of, for example, an inhibitor of the TGF-β superfamily signaling pathway. The NDF can consist essentially of, for example, A83-01, SB431542, or a combination. The NDF can consist essentially of, for example, dorsomorphin. The NDF can consist essentially of, for example, dorsomorphin and A83-01. The NDF can consist essentially of, for example, an inhibitor of the Wnt signaling pathway. The NDF can consist essentially of, for example, PNU-74654, Dickkopf, or a combination. The NDF can consist essentially of, for example, an activator of the phosphatidylinositol 3-kinase signaling pathway, an inhibitor of the TGF-β superfamily signaling pathway, and an inhibitor of the Wnt signaling pathway. The NDF can consist essentially of, for example, an activator of the phosphatidylinositol 3-kinase signaling pathway, an inhibitor of the TGF-β superfamily signaling pathway, an inhibitor of the Wnt signaling pathway, or any combination of these. The NDF can consist essentially of, for example, Midkine, Pleiotrophin, insulin-like growth factor-1, or a combination; Delta-1, Delta-2, Delta-3, Delta-4, Jagged-1, Jagged-2, or a combination; A83-01, SB431542, or a combination; dorsopmorphin; and PNU-74654, Dickkopf, or a combination. The NDF can consist essentially of, for example, Midkine, Pleiotrophin, insulin-like growth factor-1, or a combination; Delta-1, Delta-2, Delta-3, Delta-4, Jagged-1, Jagged-2, or a combination; A83-01, SB431542, or a combination; dorsopmorphin; PNU-74654, Dickkopf, or a combination; or any combination of these. The NDF can consist essentially of, for example, Midkine, A83-01, dorsopmorphin, and PNU-74654. The NDF can consist essentially of, for example, insulin-like growth factor-1, A83-01, dorsopmorphin, and PNU-74654.

The NDF can consist essentially of, for example, an activator of the Notch signaling pathway. The NDF can consist essentially of, for example, Delta-1, Delta-2, Delta-3, Delta-4, Jagged-1, Jagged-2, or a combination. The NDF can consist essentially of, for example, an activator of the phosphatidylinositol 3-kinase signaling pathway, an activator of the Notch signaling pathway, an inhibitor of the TGF-β superfamily signaling pathway, and an inhibitor of the Wnt signaling pathway. The NDF can consist essentially of, for example, an activator of the phosphatidylinositol 3-kinase signaling pathway, an activator of the Notch signaling pathway, an inhibitor of the TGF-β superfamily signaling pathway, an inhibitor of the Wnt signaling pathway, or any combination of these. The NDF can consist essentially of, for example, Midkine, Pleiotrophin, insulin-like growth factor-1, or a combination; Delta-1, Delta-2, Delta-3, Delta-4, Jagged-1, Jagged-2, or a combination; A83-01, SB431542, or a combination; dorsopmorphin; and PNU-74654, Dickkopf, or a combination. The NDF can consist essentially of, for example, Midkine, Pleiotrophin, insulin-like growth factor-1, or a combination; Delta-1, Delta-2, Delta-3, Delta-4, Jagged-1, Jagged-2, or a combination; A83-01, SB431542, or a combination; dorsopmorphin; PNU-74654, Dickkopf, or a combination; or any combination of these.

The NDF can, for example, activate the Notch signaling pathway. The NDF can comprise, for example, Delta-1, Delta-2, Delta-3, Delta-4, Jagged-1, Jagged-2, or a combination. The NDF can, for example, activate the protein kinase signaling pathway. The NDF can comprise, for example, Forskolin, dibutyryl cAMP, or a combination. The NDF can, for example, activate tyrosine kinase anaplastic lymphoma kinase (ALK). The NDF can, for example, activate insulin-like growth factor (IGF) receptor. The NDF can, for example, activate phosphatidylinositol 3-kinase. The NDF can, for example, inhibit Activin receptor-like kinase 5 (ALK5). The NDF can, for example, inhibit Activin receptor-like kinase 4 (ALK4). The NDF can, for example, inhibit Activin receptor-like kinase 7 (ALK7). The NDF can, for example, inhibit ALK5, ALK4, and ALK7. The NDF can, for example, inhibit protein phosphatase 2A (PP2A). The NDF can, for example, inhibit adenosine monophosphate-activated protein kinase (AMPK). The NDF can, for example, inhibit Bone morphogenic protein (BMP) receptor. The NDF can, for example, inhibit interaction between β-catenin and T cell factor (TCF). The NDF can, for example, activate protein-tyrosine phosphatasζ (PTPζ). The NDF can, for example, inhibit SMAD2, SMAD3, SMAD4, or a combination. The NDF can, for example, activate Notch. The NDF can, for example, inhibit SMAD1, SMAD5, SMAD8, or a combination. The NDF can, for example, inhibit Wnt binding to Frizzled. The NDF can, for example, inhibit lipoprotein receptor-related protein (LRP) binding to Frizzled. The NDF can, for example, inhibit β-catenin stabilization. The NDF can, for example, inhibit 13-catenin binding to T cell factor (TCT). The NDF can, for example, activate insulin-like growth factor-1 receptor (IGF-1R). The NDF can, for example, activate insulin receptor substrate-1 (IRS-1).

The methods can be performed wherein prior to incubating in the presence of the NDF, the stem cells are cultured in the absence of feeder cells and on an extracellular matrix. The extracellular matrix can be, for example, Matrigel™ or Geltrex™. The methods can be performed wherein prior to incubating in the presence of the NDF the stem cells are cultured on fibroblasts. The fibroblasts can be, for example, from the same species as the stem cells. The fibroblasts can be, for example, from the same subject as the stem cells. The fibroblasts can be, for example, human fibroblasts.

The methods can be performed wherein prior to incubating in the presence of the NDF, the stem cells are cultured in the presence of fibroblast growth factor 2 (FGF-2). The stem cells can also be cultured in the presence of conditioned media or chemically defined media such as mouse embryonic fibroblast-conditioned media (MEF-CM), mTeSR™ or StemPro™. The methods can be performed wherein prior to incubating in the presence of the NDF, at the same time as incubating in the presence of the NDF is initiated, or during incubating in the presence the NDF the use of FGF-2 is discontinued. The methods can be performed wherein the use of FGF-2 is discontinued at the same time as incubating in the presence of the NDF is initiated. The methods can be performed wherein the use of FGF-2 is discontinued at the same time as incubating in the presence of the NDF is initiated by replacing growth medium containing FGF-2 and lacking NDF with growth medium lacking FGF-2 and containing the NDF. The methods can be performed wherein prior to incubating in the presence of the NDF, at least a portion of the stem cells are cultured to a density of 1×10⁴ cells per square centimeter or greater.

The methods can be performed further comprising culturing the neural cells. The neural cells can be, for example, cultured on a treated polymer substrate. The treated polymer substrate can be, for example, CELLBIND™, or substrate treated with Matrigel™ Geltrex™, or fibronectin. The neural cells can be, for example, cultured in serum free conditions. The serum free conditions can comprise N2 supplement and B27 supplement. The neural cells can be, for example, cultured in the presence of fibroblast growth factor 2 (FGF-2). The neural cells can be cultured in the presence of epidermal growth factor (EGF). The neural cells can be cryopreservable. The neural cells can be, for example, passaged with Accutase or collagenase IV. The stem cells can be, for example, human stem cells. The stem cells can be, for example, embryonic stem cells (ESC). The stem cells can be, for example, derived from embryonic or fetal tissue. The stem cells can be, for example, derived from postfetal tissue. The stem cells can be, for example, derived from adult tissue. The stem cells can be, for example, derived from adult tissue. The stem cells can be, for example, induced pluripotent stem cells (iPSC). The stem cells can be, for example, derived from a subject in need of neural cells.

The neural cells can be, for example, neural stem cells. The neural cells can comprise, for example, neural stem cells. The neural cells can form, for example, neural tube-like structures. The methods can be performed further comprising differentiating the neural cells into differentiated neural cells. The neural cells can be, for example, differentiated neural cells. The neural cells can comprise, for example, neurons, astrocytes, oligodendrocytes, or a combination. The neural cells can comprise, for example, pyramidal neurons, motor neurons, spinal ventral horn motor neurons, neurons of the ventral mesencephalon, interneurons, glial cells, radial glial cells, retinal pigment epithelium, oligodendrocytes, dopamine neurons, GABA neurons, glutamate neurons, catecholinergic neurons, serotoninergic neurons, cholinergic neurons, or a combination. The neural cells can comprise, for example, pyramidal neurons, such as cortical pyramidal neurons. The neural cells can comprise, for example, neurons of the ventral mesencephalon (substantia nigra). The neural cells can comprise, for example, interneurons. The neural cells can comprise, for example, dopamine neurons, neurons that express dopamine, neurons that express molecules required for dopamine synthesis, neurons that express molecules resulting from dopamine metabolism, or a combination. The neural cells can comprise, for example, motor neurons. The neural cells can comprise, for example, spinal ventral horn motor neurons. The neural cells can comprise, for example, glial cells, including, for example, radial glial cells expressing brain lipid-binding protein (BLBP). The neural cells can comprise, for example, retinal pigment epithelium expressing, for example, the transcription factors OTX2, microphthalmia-associated transcription factor (MITE), and the tight-junction protein ZO-1. The neural cells can comprise, for example, oligodendrocytes expressing, for example, the surface marker RIP (2′,3′-cyclic nucleotide 3′-phosphodiesterase (CNPase)). The neural cells can comprise, for example, GABA neurons, neurons that express GABA, neurons that express molecules required for GABA synthesis, neurons that express molecules resulting from GABA metabolism, or a combination. The neural cells can comprise, for example, glutamate neurons, neurons that express glutamate, neurons that express molecules required for glutamate synthesis, neurons that express molecules resulting from glutamate metabolism, or a combination.

The neural cells can comprise, for example, catecholaminergic neurons, neurons that express catecholaminergically active molecules, neurons that express molecules required for synthesis of catecholaminergically active molecules, neurons that express molecules resulting from metabolism of catecholaminergically active molecules, or a combination. The neural cells can comprise, for example, serotoninergic neurons, neurons that express serotoninergically active molecules, neurons that express molecules required for synthesis of serotoninergically active molecules, neurons that express molecules resulting from metabolism of serotoninergically active molecules, or a combination. The neural cells can comprise, for example, cholinergic neurons, neurons that express molecules required for synthesis of cholinergically active molecules, neurons that express molecules resulting from metabolism of cholinergically active molecules, or a combination.

The differentiated neural cells can comprise, for example, neurons, astrocytes, oligodendrocytes, or a combination. The differentiated neural cells can comprise, for example, pyramidal neurons, motor neurons, spinal ventral horn motor neurons, neurons of the ventral mesencephalon, interneurons, glial cells, radial glial cells, retinal pigment epithelium, oligodendrocytes, dopamine neurons, GABA neurons, glutamate neurons, catecholinergic neurons, serotoninergic neurons, cholinergic neurons, or a combination. The differentiated neural cells can comprise, for example, pyramidal neurons, such as cortical pyramidal neurons. The differentiated neural cells can comprise, for example, neurons of the ventral mesencephalon (substantia nigra). The differentiated neural cells can comprise, for example, interneurons. The differentiated neural cells can comprise, for example, dopamine neurons, neurons that express dopamine, neurons that express molecules required for dopamine synthesis, neurons that express molecules resulting from dopamine metabolism, or a combination. The differentiated neural cells can comprise, for example, motor neurons. The differentiated neural cells can comprise, for example, spinal ventral horn motor neurons. The differentiated neural cells can comprise, for example, glial cells, including, for example, radial glial cells expressing BLBP. The neural cells can comprise, for example, retinal pigment epithelium expressing, for example, OTX2, MITE, and ZO-1. The neural cells can comprise, for example, oligodendrocytes expressing, for example, the surface marker RIP. The differentiated neural cells can comprise, for example, GABA neurons, neurons that express GABA, neurons that express molecules required for GABA synthesis, neurons that express molecules resulting from GABA metabolism, or a combination. The differentiated neural cells can comprise, for example, glutamate neurons, neurons that express glutamate, neurons that express molecules required for glutamate synthesis, neurons that express molecules resulting from glutamate metabolism, or a combination.

The differentiated neural cells can comprise, for example, catecholaminergic neurons, neurons that express catecholaminergically active molecules, neurons that express molecules required for synthesis of catecholaminergically active molecules, neurons that express molecules resulting from metabolism of catecholaminergically active molecules, or a combination. The differentiated neural cells can comprise, for example, serotoninergic neurons, neurons that express serotoninergically active molecules, neurons that express molecules required for synthesis of serotoninergically active molecules, neurons that express molecules resulting from metabolism of serotoninergically active molecules, or a combination. The differentiated neural cells can comprise, for example, cholinergic neurons, neurons that express cholinergically active molecules, neurons that express molecules required for synthesis of cholinergically active molecules, neurons that express molecules resulting from metabolism of cholinergically active molecules, or a combination.

The neural cells can consist essentially of, for example, neural stem cells. The neural cells can consist essentially of, for example, neurons, astrocytes, oligodendrocytes, or a combination. The neural cells can consist essentially of, for example, pyramidal neurons, motor neurons, spinal ventral horn motor neurons, neurons of the ventral mesencephalon, interneurons, glial cells, radial glial cells, retinal pigment epithelium, oligodendrocytes, dopamine neurons, GABA neurons, glutamate neurons, catecholinergic neurons, serotoninergic neurons, cholinergic neurons, or a combination. The neural cells can consist essentially of, for example, pyramidal neurons, such as cortical pyramidal neurons. The neural cells can consist essentially of, for example, neurons of the ventral mesencephalon (substantia nigra). The neural cells can consist essentially of, for example, interneurons. The neural cells can consist essentially of, for example, dopamine neurons, neurons that express dopamine, neurons that express molecules required for dopamine synthesis, neurons that express molecules resulting from dopamine metabolism, or a combination. The neural cells can consist essentially of, for example, motor neurons. The neural cells can consist essentially of, for example, spinal ventral horn motor neurons. The neural cells can consist essentially of, for example, glial cells, including, for example, radial glial cells expressing BLBP. The neural cells can consist essentially of, for example, retinal pigment epithelium expressing, for example, OTX2, MITE, and ZO-1. The neural cells can consist essentially of, for example, oligodendrocytes expressing, for example, the surface marker RIP. The neural cells can consist essentially of, for example, GABA neurons, neurons that express GABA, neurons that express molecules required for GABA synthesis, neurons that express molecules resulting from GABA metabolism, or a combination. The neural cells can consist essentially of, for example, glutamate neurons, neurons that express glutamate, neurons that express molecules required for glutamate synthesis, neurons that express molecules resulting from glutamate metabolism, or a combination.

The neural cells can consist essentially of, for example, catecholaminergic neurons, neurons that express catecholaminergically active molecules, neurons that express molecules required for synthesis of catecholaminergically active molecules, neurons that express molecules resulting from metabolism of catecholaminergically active molecules, or a combination. The neural cells can consist essentially of, for example, serotoninergic neurons, neurons that express serotoninergically active molecules, neurons that express molecules required for synthesis of serotoninergically active molecules, neurons that express molecules resulting from metabolism of serotoninergically active molecules, or a combination. The neural cells can consist essentially of, for example, cholinergic neurons, neurons that express cholinergically active molecules, neurons that express molecules required for synthesis of cholinergically active molecules, neurons that express molecules resulting from metabolism of cholinergically active molecules, or a combination.

The differentiated neural cells can consist essentially of, for example, neurons, astrocytes, oligodendrocytes, or a combination. The differentiated neural cells can consist essentially of, for example, pyramidal neurons, motor neurons, spinal ventral horn motor neurons, neurons of the ventral mesencephalon, interneurons, glial cells, radial glial cells, retinal pigment epithelium, oligodendrocytes, dopamine neurons, GABA neurons, glutamate neurons, catecholinergic neurons, serotoninergic neurons, cholinergic neurons, or a combination. The differentiated neural cells can consist essentially of, for example, pyramidal neurons, such as cortical pyramidal neurons. The differentiated neural cells can consist essentially of, for example, neurons of the ventral mesencephalon (substantia nigra). The differentiated neural cells can consist essentially of, for example, interneurons. The differentiated neural cells can consist essentially of, for example, dopamine neurons, neurons that express dopamine, neurons that express molecules required for dopamine synthesis, neurons that express molecules resulting from dopamine metabolism, or a combination. The differentiated neural cells can consist essentially of, for example, motor neurons. The differentiated neural cells can consist essentially of, for example, spinal ventral horn motor neurons. The differentiated neural cells can consist essentially of, for example, glial cells, including, for example, radial glial cells expressing BLBP. The neural cells can consist essentially of, for example, retinal pigment epithelium expressing, for example, OTX2, MITE, and ZO-1. The neural cells can consist essentially of, for example, oligodendrocytes expressing, for example, the surface marker RIP. The differentiated neural cells can consist essentially of, for example, GABA neurons, neurons that express GABA, neurons that express molecules required for GABA synthesis, neurons that express molecules resulting from GABA metabolism, or a combination. The differentiated neural cells can consist essentially of, for example, glutamate neurons, neurons that express glutamate, neurons that express molecules required for glutamate synthesis, neurons that express molecules resulting from glutamate metabolism, or a combination.

The differentiated neural cells can consist essentially of, for example, catecholaminergic neurons, neurons that express catecholaminergically active molecules, neurons that express molecules required for synthesis of catecholaminergically active molecules, neurons that express molecules resulting from metabolism of catecholaminergically active molecules, or a combination. The differentiated neural cells can consist essentially of, for example, serotoninergic neurons, neurons that express serotoninergically active molecules, neurons that express molecules required for synthesis of serotoninergically active molecules, neurons that express molecules resulting from metabolism of serotoninergically active molecules, or a combination. The differentiated neural cells can consist essentially of, for example, cholinergic neurons, neurons that express cholinergically active molecules, neurons that express molecules required for synthesis of cholinergically active molecules, neurons that express molecules resulting from metabolism of cholinergically active molecules, or a combination.

Especially useful forms of the disclosed methods involve inducing or creating balanced signaling in the phosphatidylinositol 3-kinase (PI3K) and mitogen-activated protein kinase (MAPK) signaling pathway, inhibition of the transforming growth factor-β (TGF-β) superfamily signaling pathway, and inhibition of the Wnt signaling pathway in pluripotent stem cells robustly produces neural stem cells. This inductive process is highly efficient, producing a high percentage of neural stem cells expressing Pax-6 and Nestin. Particularly useful are factors, such as Midkine and IGF-1 that activate both the PI3K and MAPK signaling pathways. This produces balanced signaling in the two pathways that aids the efficiency of neural stem cell induction. Similarly, use of factors, such as dorsomorphin and A83-01, that collectively inhibit complementary parts of the TGF-β superfamily signaling pathway, and by simultaneously inhibiting the Wnt signaling pathway, by using a factor such as PNU-74654, aids the efficiency of neural stem cell induction by reducing or eliminating production of non-ectodermal cells.

As used herein, balanced activation or signaling in the phosphatidylinositol 3-kinase (PI3K) and mitogen-activated protein kinase (MAPK) signaling pathway refers generally to the level of activation produced in pluripotent stem cells incubated in the presence of Midkine or IGF-1 and without the addition of any significant inhibitors of the PI3K signaling pathway or the MAPK signaling pathway.

While not wishing to be limited to any particular mechanism of action, the high efficiency of neuralization disclosed herein can be explained by the fact that DM, A83-01, and PNU-74654 act in multiple synergistic ways with regard to PI3K/MAPK signaling. DM not only inhibits BMP receptors but also blocks AMPK thereby disinhibiting mTOR. Similarly, through inhibition of ALK4, ALK5, and ALK7, A83-01 can indirectly reduce the activity of the phosphatase PP2a and lead to disinhibition of p70S6K, an important downstream kinase of the PI3K signaling pathway. Activation of the PI3K signaling pathway is known to inhibit GSK3β, which would thus support Wnt signaling. Blocking Wnt signaling further downstream of GSK30, such as at the level of β-catenin/TCF with PNU-74654, can antagonize the unwanted effect on GSK30, thus providing another synergistic component of the collections of NDF used in some forms of the disclosed methods.

The stem cells to be neurally induced can be any pluripotent stem cells from any source. For example, the stem cells can be embryonic stem cells or stem cells produced by somatic cell nuclear transfer. Recent findings by Yamanaka and colleagues have demonstrated that fibroblasts and other differentiated cell types (e.g. liver and stomach cells) can be reprogrammed to pluripotent cells. Such cells are referred to as induced pluripotent stem cells (iPS cells). iPS cells have been produced by, for example, introducing four transcription factors (Oct4, Sox2, Klf4, cMyc). Human iPS cells resemble and behave like hESCs in vivo and in vitro.

Methods have been discovered to generate neural tube-like structures, highly homogeneous neural stem cell populations, and more mature neural phenotypes (various neuronal cells like larger pyramidal-like neurons, dopamine neurons and motorneurons, astrocytes, oligodendrocytes) from pluripotent stem cells such as human embryonic stem cells and induced pluripotent stem cells (iPS cells). iPS cells are somatic cells like fibroblasts that have been epigenetically reprogrammed to a pluripotent embryonic stem cell-like state. The neural cells produced by the methods can be used in numerous ways. For example, the neural cells can be used for analysis of early human development and for generation of large numbers of highly pure and adherently growing neural stem cell cultures (expandable as monolayer cultures) and their differentiated progeny (various neurons, astrocytes, oligodendrocytes). The neural stem cells and differentiated neural cells can also be used, for example, for drug screening, drug design, in vitro disease modeling and clinical therapy.

The disclosed methods represent an unraveling of the process of neural induction, the fundamental step that is necessary to direct a pluripotent embryonic stem cell to a neural stem cell. The discovered methods can be, for example, more directed, defined, and faster than prior methods and can generate, for example, highly pure populations of neural stem cells and more differentiated neural progeny (for example, different neuronal populations, astrocytes, oligodendrocytes) from stem cells such as human ES cells and iPS cells. It has also been discovered that neural stem cells, such as human neural stem cells, can be efficiently expanded as monolayer cultures when plated on a treated polymer substrate, such as CELLBIND™, or substrate treated with Matrigel™, Geltrex™, or fibronectin.

The disclosed methods can be exemplified by the following. Neural induction can be achieved by, for example, simultaneously activating the phosphatidylinositol 3-kinase (PI3K) signaling pathway (with, for example, Midkine or Pleiotrophin or insulin-like growth factor-1) and blocking the TGF-beta superfamily signaling pathway and the Wnt signaling pathway. The TGF-beta superfamily can be efficiently blocked with, for example, two small molecules: A83-01 (which blocks Activin receptor-like kinase (ALK) 4, 5, and 7) and dorsomorphin (which is a BMP receptor antagonist). The Wnt signaling pathway can be blocked by, for example, the small molecule PNU-74654 or the recombinant protein Dickkopf. Midkine and insulin-like growth factor-1 also activate the MAPK signaling pathway. This protocol can generate large numbers of neural stem cells expressing Pax-6, Otx2, Nestin, or a combination from Oct4-expressing pluripotent stem cells in monolayer cultures and without the need for feeder cells. The disclosed neural induction methods generate neural stem cells directly from human embryonic stem cells under defined and controlled conditions in only 6-7 days in monolayer conditions.

The neural stems cells can be cultured to achieve neural patterning. Neural stem cells, such as the neural stem cells produced by the disclosed methods, can be, for example, directly plated on treated polymer substrate, such as CELLBIND™ (Corning), or substrate treated with Matrigel™, Geltrex™, or fibronectin, and patterned to precursors of, for example, dopamine neurons (with, for example, sonic hedgehog and fibroblast growth factor 8) or motor neurons (with, for example, retinoid acid and sonic hedgehog). Since the disclosed methods can generate highly pure adherently growing neural stem cell cultures, no additional cell enrichment strategies are necessary.

Neurally patterned cells can be used in any way differentiated neural cells can be treated and used. For example, the neural cells can be plated on regular cell culture-treated plastic dishes coated with fibronection or laminin and, for example, further differentiated to more mature neural cells.

The disclosed methods can be used as research tools for, for example, the systematic and detailed analysis of human neural differentiation in a culture dish. The neural cells can also be used for clinical applications.

The disclosed neural stem cells can be used in the disclosed methods, for example, to produce highly pure populations of neural stem cells, to expand neural stem cells in adherent cell culture conditions, and to terminally differentiate neural stem cells into various neuronal cell types, astrocytes, and oligodendrocytes. Neural stem cells generally can be characterized by, for example, expression of Pax-6. Neural stem cells can also be characterized by, for example, expression of Otx2. Neural stem cells can also be characterized by, for example, expression of Nestin. Neural stem cells can also be characterized by, for example, expression of Pax-6, Otx2, Nestin, or a combination. Neural stem cells can also be characterized by, for example, expression of Pax-6 and nestin, PLZF+, Sox1, Otx2, or a combination.

The disclosed neural stem cells and differentiated neural cells can also be used, for example, in methods to treat diseases and conditions involving neural cells and tissues. Examples of diseases and conditions that can be a target for NSC-mediated treatment or amelioration include, for example, amyotrophic lateral sclerosis (ALS; Lou Gehrig's disease, spinal muscular atrophy (SMA), Charcot-Marie-Tooth disease (CMT), Parkinson's disease, Alzheimer's disease, Huntington's disease, Rett syndrome, Autism, aging, cerebellar degeneration, spinal cord injury, stroke, and head trauma. As used herein, cell-mediated amelioration refers to, for example, rescue, protection, regeneration, or a combination of target cells. Lost or damaged neural cells or tissue can also be treated with cell replacement using the disclosed neural cells.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

### Materials

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a NDF is disclosed and discussed and a number of modifications that can be made to a number of molecules including the NDF are discussed, each and every combination and permutation of NDF and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

The disclosed methods and compositions make use of various factors (such as growth, development, signaling, and inhibitory factors). In general, such factors can be said to be, for example, used, specified, and/or defined. “Used” refers to factors that are know to be present and/or added. Factors used are those present or added, whether or not specified, defined, or required. “Specified” refers to factors are required to be used. Specified factors are those that must be present or added (for example, the presence of specified factors is not left to chance or uncertainty). Specified factors can be indicated by, for example, referring to a set of factors “comprising” particular factors. Generally, specified factors are referred to in the context of a particular stage, process, goal or function. Thus, for example, there can be specified NDF. Such specified NDF factors are used for induce, stimulate and/or mediate development of neural stem cells from pluripotent stem cells. However, other factors that may induce, stimulate and/or mediate development of neural stem cells from pluripotent stem cells can be present and other factors having other uses or functions can be present.

“Defined” refers to factors that are to be used without the use of other specified factors. Thus, defined factors are factors used without any other factors intentionally present or added. Specified factors can be indicated by, for example, referring to a set of factors “consisting of” particular factors. Generally, defined factors are referred to in the context of a particular stage, process, goal or function. Thus, for example, there can be defined NDF. Such defined NDF factors can be used to induce, stimulate and/or mediate development of neural stem cells from pluripotent stem cells. However, other factors having other uses or functions can be present. For defined NDF, other factors that may induce, stimulate and/or mediate development of neural stem cells from pluripotent stem cells generally will not be present or added.

It is understood that other conditions, materials, and factors generally required for cell growth and culturing will be used as is known by those of skill in the art. Such conditions, materials, and factors need not be specified even though they may be used in the disclosed methods and compositions. In the context of the disclosed methods and compositions, those factors, conditions and materials that are important to be present, absent, and/or at particular values (relative to general known cell growth and culturing conditions, materials and factors) in order to achieve or accomplish the stated goal (such as, for example, development of neural cells from pluripotent stem cells) generally will be specified or defined.

The disclosed methods and compositions make use of factors. “Growth factor” or “factor” refers to a substance that is effective to promote the growth and/or maintenance of cells and which, unless added to the culture medium as a supplement, is not otherwise a component of the basal medium. Put another way, a growth factor is a molecule that is not secreted by cells being cultured. Factors can be added directly or indirectly. For example, a factor can be added as a component of or in, or added to, the medium. This is direct addition of a factor. Or, for example, a factor can be supplied by cells present (such as feeder cells) or that were used to condition the medium. This is indirect addition of a factor.

Factors can include natural biomolecules that affect cell growth, cell physiology, development, and/or signaling pathways; derivatives, analogs or mimetics of such biomolecules; and compounds that that affect cell growth, cell physiology, development, and/or signaling pathways. Any or all of these types of factors (natural biomolecules, derivatives, analogs or mimetics of such biomolecules, compounds, etc.) can be used alone or in combination. Growth factors include, but are not limited to, for example, fibroblast growth factor 2 (FGF-2), acidic fibroblast growth factor (aFGF), epidermal growth factor (EGF), insulin-like growth factor-I (IGF-I), insulin-like growth factor-II (IGF-II), platelet-derived growth factor-AB (PDGF), vascular endothelial cell growth factor (VEGF), activin-A, bone morphogenic proteins (BMPs), Midkine, Pleiotrophin, insulin-like growth factor-1, Delta-1, Delta-2, Delta-3, Delta-4, Jagged-1, Jagged-2, A83-01, SB431542, dorsopmorphin, PNU-74654, Dickkopf, insulin, cytokines, chemokines, morphogents, neutralizing antibodies, other proteins, and small molecules.

For natural factors and their analogs, derivatives, and mimetics, factors from any source can be used. Thus, for example, cells (such as stem cells from which neural stem cells are to be induced) from a given species can be incubated with NDF from or derived from the same species, genus, family, order, or class, a different species, genus, family, order, or class, or a combination. It is useful to use NDF from or derived from the same species, genus, family, order, and/or class as the cells to be incubated. In particular, it is useful to use NDF from or derived from the same species as the cells to be incubated. As another example, cells (such as stem cells from which neural stem cells are to be induced) from a given species can be pre-incubated with pre-incubation factors from or derived from the same species, genus, family, order, or class, a different species, genus, family, order, or class, or a combination. It is useful to use pre-incubation factors from or derived from the same species, genus, family, order, and/or class as the cells to be pre-incubated. In particular, it is useful to use pre-incubation from or derived from the same species as the cells to be pre-incubated. As another example, cells (such as neural cells produced in the disclosed methods) from a given species can be cultured with factors from or derived from the same species, genus, family, order, or class, a different species, genus, family, order, or class, or a combination. It is useful to use factors from or derived from the same species, genus, family, order, and/or class as the cells to be cultured. In particular, it is useful to use factors from or derived from the same species as the cells to be cultured.

### A. Neural Development Factors

Neural Development Factors (NDF) are factors used to induce, stimulate and/or mediate development of neural stem cells from pluripotent stem cells. As used herein, “Neural Development Factors” refers to any factor or combination of factors used during the incubation that produces the neural cells. Thus, the Neural Development Factors include activating factors, inhibiting factors, or a combination. It is understood that NDF are those factors that are required and/or intended to induce, stimulate and/or mediate development of neural stem cells from pluripotent stem cells. Additional factors may be present or used during incubation with NDF, but such additional factors need not be NDF and can be referred to as non-NDF. Both NDF and non-NDF can be used, specified, and/or defined. NDF factors used are those present or added, whether or not specified, defined, or required. Specified NDF factors are those that must be present or added. Defined NDF factors are factors used without any other factors intentionally present or added. Such defined NDF factors are used for induce, stimulate and/or mediate development of neural stem cells from pluripotent stem cells. However, other factors having other uses or functions can be present.

It is also understood that NDF can be used alone with no other factors present and/or used. Where a defined set of factors is stated as being used (by stating, for example, that the factors “consist of” the defined set of factors), it is intended that no other factors are added (other than those factors that may be present in the medium or produced by the stem cells and/or feeder cells (if present)). Thus, other factors may be present that are present in the medium or produced by the cells present, but only the defined set of factors are provided or accounted for. In this case, the other factors that happen to be present would be considered non-NDF. For example, numerous general growth factors may be present and need not be excluded when, for example, a defined set of NDF is specified.

It is contemplated that some of the specified factors can be provided by the cells or medium used. Thus, for example, one or more of the NDF specified can be provided by the cells or medium, can be provided separately and/or in addition to the cells or medium, or by a combination. Other factors that may be added or present in or provided by the cells or medium would be non-NDF. As discussed, such non-NDF may be present that are present in the medium or produced by the cells present even if a defined set of NDF is used.

It has been discovered that providing the appropriate cell culture conditions and modulation of specific signaling pathways mediates differentiation of pluripotent stem cells (PSCs). Based on this, the disclosed methods and compositions make use of NDF that modulate one or more signaling pathways discovered to be involved in the differentiation of PSCs into neural cells. The disclosed methods provide for neural induction and production of neural cells from pluripotent stem cells, including, for example, embryonic stem cells and reprogrammed somatic cells with acquired pluripotency.

Useful signaling pathways to be modulated by NDF include the phosphatidylinositol 3-kinase signaling pathway, the mitogen-activated protein kinase (MAPK) signaling pathway, the TGF-β superfamily signaling pathway, the Wnt signaling pathway, or any combination of these. Thus, the NDF can include, for example, any factor that modulates one of these signaling pathways. It is particularly useful for NDF that activate the phosphatidylinositol 3-kinase signaling pathway, inhibit the TGF-β superfamily signaling pathway, inhibit the Wnt signaling pathway, or any combination of these. Different factors can be used to modulate different pathways. It is also useful for NDF that activate the phosphatidylinositol 3-kinase signaling pathway, activate the MAPK signaling pathway, inhibit the TGF-β superfamily signaling pathway, inhibit the Wnt signaling pathway, or any combination of these. Different factors can be used to modulate different pathways. In some cases, particular factors can be capable of modulating two or more signaling pathways.

The NDF can, for example, activate the phosphatidylinositol 3-kinase (PI3K) signaling pathway. The NDF can, for example, activate the mitogen-activated protein kinase (MAPK) signaling pathway. The NDF can, for example, activate the phosphatidylinositol 3-kinase signaling pathway and can activate the MAPK signaling pathway. The NDF can, for example, activate the phosphatidylinositol 3-kinase signaling pathway and the MAPK signaling pathway. The NDF can, for example, activate the phosphatidylinositol 3-kinase signaling pathway and the MAPK signaling pathway in a balanced manner. The NDF can, for example, inhibit the TGF-β superfamily signaling pathway. The NDF can, for example, inhibit the Wnt signaling pathway. The NDF can, for example, activate the phosphatidylinositol 3-kinase signaling pathway, inhibit the TGF-β superfamily signaling pathway, and inhibit the Wnt signaling pathway. The NDF can, for example, activate the phosphatidylinositol 3-kinase signaling pathway, inhibit the TGF-β superfamily signaling pathway, inhibit the Wnt signaling pathway, or any combination of these.

The NDF can comprise, for example, an activator of the phosphatidylinositol 3-kinase signaling pathway. The NDF can comprise, for example, an activator of the MAPK signaling pathway. The NDF can comprise, for example, an activator of the phosphatidylinositol 3-kinase signaling pathway and the MAPK signaling pathway. The NDF can comprise, for example, Midkine, Pleiotrophin, insulin-like growth factor-1, or a combination. The NDF can comprise, for example, Midkine, insulin-like growth factor-1, or a combination. The NDF can comprise, for example, Midkine. The NDF can comprise, for example, an activator of the phosphatidylinositol 3-kinase signaling pathway and an activator of the MAPK signaling pathway. The NDF can comprise, for example, an inhibitor of the TGF-β superfamily signaling pathway. The NDF can comprise, for example, A83-01, SB431542, or a combination. The NDF can comprise, for example, dorsomorphin. The NDF can comprise, for example, dorsomorphin and A83-01. The NDF can comprise, for example, an inhibitor of the Wnt signaling pathway. The NDF can comprise, for example, PNU-74654, Dickkopf, or a combination. The NDF can comprise, for example, an activator of the phosphatidylinositol 3-kinase signaling pathway, an inhibitor of the TGF-β superfamily signaling pathway, and an inhibitor of the Wnt signaling pathway. The NDF can comprise, for example, an activator of the phosphatidylinositol 3-kinase signaling pathway, an inhibitor of the TGF-β superfamily signaling pathway, an inhibitor of the Wnt signaling pathway, or any combination of these. The NDF can comprise, for example, Midkine, Pleiotrophin, insulin-like growth factor-1, or a combination; A83-01, SB431542, or a combination; dorsopmorphin; and PNU-74654, Dickkopf, or a combination. The NDF can comprise, for example, Midkine, Pleiotrophin, insulin-like growth factor-1, or a combination; A83-01, SB431542, or a combination; dorsopmorphin; PNU-74654, Dickkopf, or a combination; or any combination of these. The NDF can comprise, for example, Midkine, A83-01, dorsopmorphin, and PNU-74654. The NDF can comprise, for example, insulin-like growth factor-1, A83-01, dorsopmorphin, and PNU-74654.

The NDF can consist of, for example, an activator of the phosphatidylinositol 3-kinase signaling pathway. The NDF can consist of, for example, an activator of the MAPK signaling pathway. The NDF can consist of, for example, an activator of the phosphatidylinositol 3-kinase signaling pathway and the MAPK signaling pathway. The NDF can consist of, for example, Midkine, Pleiotrophin, insulin-like growth factor-1, or a combination. The NDF can consist of, for example, Midkine, insulin-like growth factor-1, or a combination. The NDF can consist of, for example, Midkine. The NDF can consist of, for example, an activator of the phosphatidylinositol 3-kinase signaling pathway and an activator of the MAPK signaling pathway. The NDF can consist of, for example, an inhibitor of the TGF-β superfamily signaling pathway. The NDF can consist of, for example, A83-01, SB431542, or a combination. The NDF can consist of, for example, dorsomorphin. The NDF can consist of, for example, dorsomorphin and A83-01. The NDF can consist of, for example, an inhibitor of the Wnt signaling pathway. The NDF can consist of, for example, PNU-74654, Dickkopf, or a combination. The NDF can consist of, for example, an activator of the phosphatidylinositol 3-kinase signaling pathway, an inhibitor of the TGF-β superfamily signaling pathway, and an inhibitor of the Wnt signaling pathway. The NDF can consist of, for example, an activator of the phosphatidylinositol 3-kinase signaling pathway, an inhibitor of the TGF-β superfamily signaling pathway, an inhibitor of the Wnt signaling pathway, or any combination of these. The NDF can consist of, for example, Midkine, Pleiotrophin, insulin-like growth factor-1, or a combination; A83-01, SB431542, or a combination; dorsopmorphin; and PNU-74654, Dickkopf, or a combination. The NDF can consist of, for example, Midkine, Pleiotrophin, insulin-like growth factor-1, or a combination; A83-01, SB431542, or a combination; dorsopmorphin; PNU-74654, Dickkopf, or a combination; or any combination of these. The NDF can consist of, for example, Midkine, A83-01, dorsopmorphin, and PNU-74654. The NDF can consist of, for example, insulin-like growth factor-1, A83-01, dorsopmorphin, and PNU-74654.

The NDF can consist essentially of, for example, an activator of the phosphatidylinositol 3-kinase signaling pathway. The NDF can consist essentially of, for example, an activator of the MAPK signaling pathway. The NDF can consist essential of, for example, an activator of the phosphatidylinositol 3-kinase signaling pathway and the MAPK signaling pathway. The NDF can consist essentially of, for example, Midkine, Pleiotrophin, insulin-like growth factor-1, or a combination. The NDF can consist essentially of, for example, Midkine, insulin-like growth factor-1, or a combination. The NDF can consist essentially of, for example, Midkine. The NDF can consist essentially of, for example, an activator of the phosphatidylinositol 3-kinase signaling pathway and an activator of the MAPK signaling pathway. The NDF can consist essentially of, for example, an inhibitor of the TGF-β superfamily signaling pathway. The NDF can consist essentially of, for example, A83-01, SB431542, or a combination. The NDF can consist essentially of, for example, dorsomorphin. The NDF can consist essentially of, for example, dorsomorphin and A83-01. The NDF can consist essentially of, for example, an inhibitor of the Wnt signaling pathway. The NDF can consist essentially of, for example, PNU-74654, Dickkopf, or a combination. The NDF can consist essentially of, for example, an activator of the phosphatidylinositol 3-kinase signaling pathway, an inhibitor of the TGF-β superfamily signaling pathway, and an inhibitor of the Wnt signaling pathway. The NDF can consist essentially of, for example, an activator of the phosphatidylinositol 3-kinase signaling pathway, an inhibitor of the TGF-β superfamily signaling pathway, an inhibitor of the Wnt signaling pathway, or any combination of these. The NDF can consist essentially of, for example, Midkine, Pleiotrophin, insulin-like growth factor-1, or a combination; Delta-1, Delta-2, Delta-3, Delta-4, Jagged-1, Jagged-2, or a combination; A83-01, SB431542, or a combination; dorsopmorphin; and PNU-74654, Dickkopf, or a combination. The NDF can consist essentially of, for example, Midkine, Pleiotrophin, insulin-like growth factor-1, or a combination; Delta-1, Delta-2, Delta-3, Delta-4, Jagged-1, Jagged-2, or a combination; A83-01, SB431542, or a combination; dorsopmorphin; PNU-74654, Dickkopf, or a combination; or any combination of these. The NDF can consist essentially of, for example, Midkine, A83-01, dorsopmorphin, and PNU-74654. The NDF can consist essentially of, for example, insulin-like growth factor-1, A83-01, dorsopmorphin, and PNU-74654.

The NDF can also include other factors. Such other factors can affect, for example, the phosphatidylinositol 3-kinase signaling pathway, the TGF-β superfamily signaling pathway, or the Wnt signaling pathway. Such other factors can affect other signaling pathways. Such other factors can affect, for example, the Notch signaling pathway or the protein kinase signaling pathway. The NDF can include, for example, any factor that activates the Notch signaling pathway. The NDF can, for example, activate the Notch signaling pathway. The NDF can comprise, for example, an activator of the Notch signaling pathway. The NDF can comprise, for example, Delta-1, Delta-2, Delta-3, Delta-4, Jagged-1, Jagged-2, or a combination. The NDF can include, for example, any factor that activates the protein kinase signaling pathway. The NDF can, for example, activate the protein kinase signaling pathway. The NDF can comprise, for example, an activator of the protein kinase signaling pathway. The NDF can comprise, for example, Forskolin, dibutyryl cAMP, or a combination.

The NDF can, for example, activate tyrosine kinase anaplastic lymphoma kinase (ALK). The NDF can, for example, activate insulin-like growth factor (IGF) receptor. The NDF can, for example, activate phosphatidylinositol 3-kinase. The NDF can, for example, inhibit Activin receptor-like kinase 5 (ALK5). The NDF can, for example, inhibit Activin receptor-like kinase 4 (ALK4). The NDF can, for example, inhibit Activin receptor-like kinase 7 (ALK7). The NDF can, for example, inhibit ALK5, ALK4, and ALK7. The NDF can, for example, inhibit protein phosphatase 2A (PP2A). The NDF can, for example, inhibit adenosine monophosphate-activated protein kinase (AMPK). The NDF can, for example, inhibit Bone morphogenic protein (BMP) receptor. The NDF can, for example, inhibit interaction between β-catenin and T cell factor (TCF). The NDF can, for example, activate protein-tyrosine phosphatasζ (PTPζ). The NDF can, for example, inhibit SMAD2, SMAD3, SMAD4, or a combination. The NDF can, for example, activate Notch. The NDF can, for example, inhibit SMAD1, SMAD5, SMAD8, or a combination. The NDF can, for example, inhibit Wnt binding to Frizzled. The NDF can, for example, inhibit lipoprotein receptor-related protein (LRP) binding to Frizzled. The NDF can, for example, inhibit β-catenin stabilization. The NDF can, for example, inhibit β-catenin binding to T cell factor (TCT). The NDF can, for example, activate insulin-like growth factor-1 receptor (IGF-1R). The NDF can, for example, activate insulin receptor substrate-1 (IRS-1).

### B. Pre-incubation Factors

Pluripotent stem cells generally can be grown, cultured, and/or maintained prior to incubation with NDF. This is referred to herein as “pre-incubation.” Pre-incubation generally can be carried out with any factors and under any conditions known or used to grow, culture, and/or maintain pluripotent stem cell. A variety of such conditions are known. For example, fibroblast growth factor 2 (FGF-2) together with conditioned medium derived from mouse embryonic fibroblasts (MEFs) or commercially available chemically defined media (mTeSR™ or StemPro™) are widely used to grow pluripotent stem cells. Factors used for pre-incubation can be referred to as pre-incubation factors (PFs). Useful factors are those that maintain high pluripotency in the stem cells. Some growth, culturing, and/or maintenance conditions are particularly useful for pre-incubation prior to neural induction. For example, it is useful to use fibroblast growth factor 2 (FGF-2) as a pre-incubation factor. The stem cells can also be cultured in the presence of conditioned media or chemically defined media such as mouse embryonic fibroblast-conditioned media (MEF-CM), mTeSR™ or StemPro™.

### C. Neural Culturing Factors

Once neural stem cells are produced, the neural stem cells can be grown, cultured, and/or maintained. This is referred to herein as “culturing” or “neural culturing.” For example, the neural stem cells can be cultured prior to use and/or prior to further differentiation. Culturing generally can be carried out with any factors and under any conditions known or used to grow, culture, and/or maintain stem cells and/or neural stem cells. A variety of such conditions are known. Factors used for culturing can be referred to as “Neural Culturing Factors” (NCFs). Some growth, culturing, and/or maintenance conditions are particularly useful for culturing of neural stem cells. For example, it is useful to use fibroblast growth factor 2 (FGF-2) as a culturing factor. Epidermal growth factor (EGF) can also be sued as a culturing factor. It can also be useful to use serum free conditions and to use N2 supplement and B27 supplement.

The disclosed methods, compositions, and factors can be used to culture any neural stem cells from any source. The disclosed methods of culturing neural stem cells are not limited to culturing neural stem cells produced in the disclosed methods.

### D. Neural Cell Differentiation Factors

Differentiated neural cells can be produced from neural stem cells by incubating or culturing neural stem cells under conditions that induce, stimulated and/or mediate development of differentiated neural cells. For example, neural stem cells can be differentiated into differentiated neural cells, neurons, astrocytes, oligodendrocytes, dopamine neurons, or motor neurons. The neural cells can comprise, for example, pyramidal neurons, motor neurons, spinal ventral horn motor neurons, neurons of the ventral mesencephalon, interneurons, glial cells, radial glial cells, retinal pigment epithelium, oligodendrocytes, dopamine neurons, GABA neurons, glutamate neurons, catecholinergic neurons, serotoninergic neurons, cholinergic neurons, or a combination. The neural cells can form, for example, neural tube-like structures. Differentiation generally can be carried out with any factors and under any conditions known or used to differentiate neural cells. A variety of such conditions are known. The disclosed methods and neural stem cells can also be used to identify factors and conditions that induce, stimulated and/or mediate development of differentiated neural cells. Factors used for differentiating neural cells from neural stem cells can be referred to as “Neural Cell Differentiation Factors” (NCDFs). For example, it is useful to use retinoic acid, sonic hedgehog, fibroblast growth factor 8 (FGF-8), brain-derived neurotrophic factor (BDNF), glial-derived neurotrophic factor (GDNF), ascorbic acid, N2 supplement, and B27 supplement (Chambers et al., 2009).

### E. Stem Cells

Stem cells are defined (Gilbert, (1994) Developmental Biology, 4th Ed. Sinauer Associates, Inc. Sunderland, Mass., p. 354) as cells that are “capable of extensive proliferation, creating more stem cells (self-renewal) as well as more differentiated cellular progeny.” These characteristics can be referred to as stem cell capabilities. Pluripotent stem cells (also referred to as pluripotential stem cells), adult stem cells, blastocyst-derived stem cells, gonadal ridge-derived stem cells, teratoma-derived stem cells, totipotent stem cells, multipotent stem cells, embryonic stem cells (ES), embryonic germ cells (EG), and embryonic carcinoma cells (EC) are all examples of stem cells.

Stem cells can have a variety of different properties and categories of these properties. For example in some forms stem cells are capable of proliferating for at least 10, 15, 20, 30, or more passages in an undifferentiated state. In some forms the stem cells can proliferate for more than a year without differentiating. Stem cells can also maintain a normal karyotype while proliferating and/or differentiating. Stem cells can also be capable of retaining the ability to differentiate into mesoderm, endoderm, and ectoderm tissue, including germ cells, eggs and sperm. Some stem cells can also be cells capable of indefinite proliferation in vitro in an undifferentiated state. Some stem cells can also maintain a normal karyotype through prolonged culture. Some stem cells can maintain the potential to differentiate to derivatives of all three embryonic germ layers (endoderm, mesoderm, and ectoderm) even after prolonged culture. Some stem cells can form any cell type in the organism. Some stem cells can form embryoid bodies under certain conditions, such as growth on media which do not maintain undifferentiated growth. Some stem cells can form chimeras through fusion with a blastocyst, for example.

Some stem cells can be defined by a variety of markers. For example, some stem cells express alkaline phosphatase. Some stem cells express SSEA-1, SSEA-3, SSEA-4, TRA-1-60, and/or TRA-1-81. Some stem cells do not express SSEA-1, SSEA-3, SSEA-4, TRA-1-60, and/or TRA-1-81. Some stem cells express Oct 4 and Nanog (Rodda et al., J. Biol. Chem. 280, 24731-24737 (2005); Chambers et al., Cell 113, 643-655 (2003)). It is understood that some stem cells will express these at the mRNA level, and still others will also express them at the protein level, on for example, the cell surface or within the cell.

It is understood that stem cells can have any combination of any stem cell property or category or categories and properties discussed herein. For example, some stem cells can express alkaline phosphatase, not express SSEA-1, proliferate for at least 20 passages, and be capable of differentiating into any cell type. Another set of stem cells, for example, can express SSEA-1 on the cell surface, and be capable of forming endoderm, mesoderm, and ectoderm tissue and be cultured for over a year without differentiation. Another set of stem cells, for example, could be pluripotent stem cells that express SSEA-1. Another set of stem cells, for example, could be blastocyst-derived stem cells that express alkaline phosphatase.

Stem cells can be cultured using any culture means which promotes the properties of the desired type of stem cell. For example, stem cells can be cultured in the presence of fibroblast growth factor 2 (FGF-2; also known as basic fibroblast growth factor), leukemia inhibitory factor, membrane associated steel factor, and soluble steel factor which will produce pluripotent embryonic stem cells. See U.S. Pat. Nos. 5,690,926; 5,670,372, and 5,453,357, which are all incorporated herein by reference for material at least related to deriving and maintaining pluripotent embryonic stem cells in culture. Stem cells can also be cultured on embryonic fibroblasts and dissociated cells can be re-plated on embryonic feeder cells. See for example, U.S. Pat. Nos. 6,200,806 and 5,843,780 which are herein incorporated by reference at least for material related to deriving and maintaining stem cells.

For culturing stem cells, feeder cells can be used (as described elsewhere herein, stem cells can also be cultured in the absence of feeder cells). “Feeder cells” are cells on which cells to be cultured that differ from the cells to be cultured can be plated and which provide a milieu conducive to the growth of the cells. The use of feeder cells is well known to those of skill in the art. Feeder cells from any source can be used. Thus, for example, cells from a given species can be cultured with feeder cells from or derived from the same species, genus, family, order, or class, a different species, genus, family, order, or class, or a combination. It is useful to use feeder cells from or derived from the same species, genus, family, order, and/or class as the stem cells to be cultured. In particular, it is useful to use feeder cells from or derived from the same species as the stem cells to be cultured. As another example, stem cells from a given species can be cultured with feeder cells from or derived from the same species, genus, family, order, or class, a different species, genus, family, order, or class, or a combination. It is useful to use feeder cells from or derived from the same species, genus, family, order, and/or class as the stem cells to be cultured. In particular, it is useful to use feeder cells from or derived from the same species as the stem cells to be cultured. As another example, stem cells from a given species can be cultured with feeder cells from or derived from the same species, genus, family, order, or class, a different species, genus, family, order, or class, or a combination. It is useful to use feeder cells from or derived from the same species, genus, family, order, and/or class as the stem cells to be cultured. In particular, it is useful to use feeder cells from or derived from the same species as the stem cells to be cultured. Fibroblasts are particularly useful as feeder cells for culturing stem cells.

One category of stem cells is a pluripotent stem cell. A pluripotent stem cell as used herein means a cell which can give rise to many differentiated cell types in an embryo or adult, including the germ cells (sperm and eggs). Pluripotent stem cells are also capable of self-renewal. Thus, these cells can not only populate the germ line and give rise to a plurality of terminally differentiated cells which comprise the adult specialized organs, but also are able to regenerate themselves.

One category of stem cells is a pluripotent embryonic stem cell. A pluripotent embryonic stem cell as used herein means a cell which can give rise to many differentiated cell types in an embryo or adult, including the germ cells (sperm and eggs). Pluripotent embryonic stem cells are also capable of self-renewal. Thus, these cells not only populate the germ line and give rise to a plurality of terminally differentiated cells which comprise the adult specialized organs, but also are able to regenerate themselves.

One category of stem cells is an induced pluripotent stem cell (iPSC). Induced pluripotent stem cells are differentiated somatic cells that have been induced to produce cells having pluripotency and growth ability similar to those of ES cells. Such cells are useful because they do not require the use of embryonic or fetal tissue. Such cells are also useful because they can be generated from somatic cells of a subject in need of stems cells, neural stem cells, or neural cells such as those disclosed herein. Techniques for producing iPSC are known. For example, U.S. Patent Application Publication Nos. 20090068742, 20090047263, 20080293143, and 20080233610 describe techniques for producing iPSC.

One category of stem cells are cells which are capable of self renewal and which can differentiate into cell types of the mesoderm, ectoderm, and endoderm, but which do not give rise to germ cells, sperm or egg.

Another category of stem cells are stem cells which are capable of self renewal and which can differentiate into cell types of the mesoderm, ectoderm, and endoderm, but which do not give rise to placenta cells.

Another category of stem cells is an adult stem cell which is any type of stem cell that is not derived from an embryo or fetus. Typically, these stem cells have a limited capacity to generate new cell types and are committed to a particular lineage, although adult stem cells capable of generating all three cell types have been described (for example, U.S. Patent Application Publication No 20040107453 by Furcht, et al. published Jun. 3, 2004 and PCT/US02/04652, which are both incorporated by reference at least for material related to adult stem cells and culturing adult stem cells). An example of an adult stem cell is the multipotent hematopoietic stem cell, which forms all of the cells of the blood, such as erythrocytes, macrophages, T and B cells. Cells such as these are referred to as “pluripotent hematopoietic stem cell” for its pluripotency within the hematopoietic lineage. Another example of an adult stem cell is a neural stem cell. A pluripotent adult stem cell is an adult stem cell having pluripotent capabilities (See for example, U.S. Patent Publication no. 20040107453, which is U.S. patent application Ser. No. 10/467,963).

Another category of stem cells is a blastocyst-derived stem cell which is a pluripotent stem cell which was derived from a cell which was obtained from a blastocyst prior to the, for example, 64, 100, or 150 cell stage. Blastocyst-derived stem cells can be derived from the inner cell mass of the blastocyst and are the cells commonly used in transgenic mouse work (Evans and Kaufman, (1981) Nature 292:154-156; Martin, (1981) Proc. Natl. Acad. Sci. 78:7634-7638). Blastocyst-derived stem cells isolated from cultured blastocysts can give rise to permanent cell lines that retain their undifferentiated characteristics indefinitely. Blastocyst-derived stem cells can be manipulated using any of the techniques of modern molecular biology, then re-implanted in a new blastocyst. This blastocyst can give rise to a full term animal carrying the genetic constitution of the blastocyst-derived stem cell. (Misra and Duncan, (2002) Endocrine 19:229-238). Such properties and manipulations are generally applicable to blastocyst-derived stem cells. It is understood blastocyst-derived stem cells can be obtained from pre or post implantation embryos and can be referred to as that there can be pre-implantation blastocyst-derived stem cells and post-implantation blastocyst-derived stem cells respectively.

Another category of stem cells is a gonadal ridge-derived stem cell which is a pluripotent stem cell which was derived from a cell which was obtained from, for example, a human embryo or fetus at or after the 6, 7, 8, 9, or 10 week, post ovulation, developmental stage. Alkaline phosphatase staining occurs at the 5-6 week stage. Gonadal ridge-derived stem cell can be derived from the gonadal ridge of, for example, a 6-10 week human embryo or fetus from gonadal ridge cells.

Another category of stem cells are embryo-derived stem cells which are derived from embryos of 150 cells or more up to 6 weeks of gestation. Typically embryo-derived stem cells will be derived from cells that arose from the inner cell mass cells of the blastocyst or cells which will be come gonadal ridge cells, which can arise from the inner cell mass cells, such as cells which migrate to the gonadal ridge during development.

Other sets of stem cells are embryonic stem cells, (ES cells), embryonic germ cells (EG cells), and embryonic carcinoma cells (EC cells).

Also disclosed is another category of stem cells called teratoma-derived stem cells which are stem cells which are derived from a teratocarcinoma and can be characterized by the lack of a normal karyotype. Teratocarcinomas are unusual tumors that, unlike most tumors, are comprised of a wide variety of different tissue types. Studies of teratocarcinoma suggested that they arose from primitive gonadal tissue that had escaped the usual control mechanisms. Such properties and manipulations are generally applicable to teratoma-derived stem cells.

Stem cells can also be classified by their potential for development. One category of stem cells are stem cells that can grow into an entire organism. Another category of stem cells are stem cells (which have pluripotent capabilities as defined above) that cannot grow into a whole organism, but can become any other type of cell in the body. Another category of stem cells are stem cells that can only become particular types of cells: e.g. blood cells, or bone cells. Such stem cells can be referred to herein cell type-specific stem cells or cell lineage-specific stem cells. Neural stem cells and hematopoietic stem cell are examples. Cell type-specific stem cells can be derived from any type of stem cell, and in particular, from any type of pluripotent stem cell. For example, neural stem cells can be derived from embryonic stem cells or induced pluripotent stem cells. Other categories of stem cells include totipotent, pluripotent, and multipotent stem cells. Totipotent cells are capable of developing into an organism, including all of the cells of an organism, and the extra embryonic tissue. For example, totipotent cells can differentiate into any cell type, including pluripotent, multipotent, and fully differentiated cells (i.e., cells no longer capable of differentiation into various cell types), such as, without limitation, embryonic stem cells, neural stem cells, bone marrow stem cells, hematopoietic stem cells, cardiomyocytes, neuron, astrocytes, muscle cells, and connective tissue cells.

The disclosed methods and compositions are generally described by reference to “stem cells” or “pluripotent stem cells.” However, the disclosed methods are not limited to use of stem cells and pluripotent stem cells. It is specifically contemplated that the disclosed methods and compositions can use or comprise any type or category of stem cell, such as adult stem cells, blastocyst-derived stem cells, gonadal ridge-derived stem cells, teratoma-derived stem cells, totipotent stem cells, and multipotent stem cells, or stem cells having any of the properties described herein. The use of any type or category of stem cell, both alone and in any combination, with or in the disclosed methods and compositions is specifically contemplated and described.

A “normal” stem cell refers to a stem cell (or its progeny) that does not exhibit an aberrant phenotype or have an aberrant genotype, and thus can give rise to the full range of cells that be derived from such a stem cell. In the context of a totipotent stem cell, for example, the cell could give rise to, for example, an entire, normal animal that is healthy. In contrast, an “abnormal” stem cell refers to a stem cell that is not normal, due, for example, to one or more mutations or genetic modifications or pathogens. Thus, abnormal stem cells differ from normal stem cells.

A “primate-derived stem cell” or “primate stem cell” is a stem cell obtained from a primate species, including humans and monkeys, and includes genetically modified stem cells.

“Substantially undifferentiated” means that population of stem cells (e.g., embryonic stem cells or iPSC) contains at least about 50%, preferably at least about 60%, 70%, or 80%, and even more preferably, at least about 90%, undifferentiated, stem cells. Fluorescence-activated cell sorting using labeled antibodies or reporter genes/proteins (e.g., enhanced green fluorescence protein [EGFP]) to one or more markers indicative of a desired undifferentiated state (e.g., a primordial state) can be used to determine how many cells of a given stem cell population are undifferentiated. For purposes of making this assessment, one or more of cell surface markers correlated with an undifferentiated state (e.g., Oct-4, SSEA-4, Tra-1-60, and Tra-1-81) can be detected. Telomerase reverse transcriptase (TERT) activity and alkaline phosphatase can also be assayed. In the context of, for example, embryonic stem cells or iPSC, positive and/or negative selection can be used to detect, for example, by immuno-staining or employing a reporter gene (e.g., EGFP), the expression (or lack thereof) of certain markers (e.g., Oct-4, SSEA-4, Tra-1-60, Tra-1-81, SSEA-1, SSEA-3, nestin, telomerase, Myc, p300, and Tip60 histone acetyltransferases, and alkaline phosphatase activity) or the presence of certain post-translational modifications (e.g., acetylated histones), thereby facilitating assessment of the state of self-renewal or differentiation of the cells.

1. Neural Stem Cells

Neural stem cells, which can also be referred to as neuropoietic stem cells and pluripotent neuropoietic stems cells, can form any type or neural cell. Neural stem cells can have a variety of different properties and categories of these properties. For example in some forms neural stem cells are capable of proliferating for at least 10, 15, 20, 30, or more passages in an undifferentiated state. In some forms the neural stem cells can proliferate for more than a year without differentiating. Neural stem cells can also maintain a normal karyotype while proliferating and/or differentiating. Some neural stem cells can also be cells capable of indefinite proliferation in vitro in an undifferentiated state. Some neural stem cells can also maintain a normal karyotype through prolonged culture. In the context of cell type-specific stem cells, such as neural stem cells, an undifferentiated state refers to the cell type-specific stem cell state, which technically can be considered partially differentiated or committed. This is done merely for convenience.

Some stem cells can be defined by a variety of markers. For example, neural stem cells can express one or more of Pax-6, Otx1, Otx2, Nestin, PSA-NCAM, and p75 Neurotrophin R (NTR). It is understood that some neural stem cells will express these at the mRNA level, and still others will also express them at the protein level, on for example, the cell surface or within the cell. It is understood that neural stem cells can have any combination of any neural stem cell property or category or categories and properties discussed herein.

Neural stem cells can be cultured using any culture means which promotes the properties of neural stem cells. For example, neural stem cells can be cultured in the presence of fibroblast growth factor 2 (FGF-2). The neural cells can be cultured in the presence of epidermal growth factor (EGF). The neural cells can be cryopreservable. Stem cells can also be cultured on fibroblasts and dissociated cells can be re-plated on feeder cells.

For culturing neural stem cells, feeder cells can be used (as described elsewhere herein, neural stem cells can also be cultured in the absence of feeder cells). Feeder cells can be from any source can be used. Thus, for example, neural stem cells from a given subject or species can be cultured with feeder cells from or derived from the same subject, species, genus, family, order, or class, a different species, genus, family, order, or class, or a combination. It is useful to use feeder cells from or derived from the same subject, species, genus, family, order, and/or class as the neural stem cells to be cultured. Fibroblasts are particularly useful as feeder cells for culturing neural stem cells. In particular, it is useful to use feeder cells from or derived from the same species as the neural stem cells to be cultured. As another example, neural stem cells from a given species can be cultured with feeder cells from or derived from the same species, genus, family, order, or class, a different species, genus, family, order, or class, or a combination.

One category of neural stem cells is a pluripotent neuropoietic stem cell. A pluripotent neuropoietic stem cell as used herein means a cell which can give rise to many differentiated neural cell types. Neural stem cells are capable of self-renewal. One category of neural stem cells are neural stem cells which are capable of self renewal. Another category of neural stem cells is an adult neural stem cell which is any type of neural stem cell that is not derived from an embryo or fetus. An example of an adult neural stem cell is the multipotent neuropoietic stem cell, which forms all types of neural cells. Cells such as these can be referred to as “pluripotent neuropoietic stem cell” for their pluripotency within the neuropoietic lineage.

Paired box gene 6 (aniridia, keratitis), also known as Pax-6, is a gene in humans and other animals. Pax-6 is the most researched of the Pax genes and appears throughout the literature as a “master control” gene for the development of eyes and other sensory organs, certain neural and epidermal tissues as well as other homologous structures, usually derived from ectodermal tissues. This transcription factor is most famous for its use in the interspecifically induced expression of ectopic eyes and is of medical importance because heterozygous mutants produce a wide spectrum of ocular defects such as Aniridia in humans. Pax-6 protein function is highly conserved across bilaterian species, for instance mouse Pax-6 can trigger eye development in Drosophila melanogaster.

Genomic organization of the Pax-6 locus varies considerably among species, including the number and distribution of exons, cis-regulatory elements, and transcription start sites. The first work on genomic organization was performed in quail, but the picture of the mouse locus is the most complete to date. This consists of 2 confirmed promoters (P0 and P1), 16 exons, and at least 6 enhancers. The 16 confirmed exons are numbered 0 through 13 with the additions of exon a located between exons 4 and 5, and the alternatively spliced exon 5a. Each promoter is associated with its own proximal exon (exon 0 for P0, exon 1 for P1) resulting in transcripts which are alternatively spliced in the 5′ un-translated region.

The vertebrate Pax-6 locus encodes at least three different protein isoforms, these being the canonical Pax-6, Pax-6(5a), and Pax-6(ΔPD). The canonical Pax-6 protein contains an N-terminal paired domain, connected by a linker region to a paired-type homeodomain, and a prolein/serine/threonine (P/S/T)-rich C-terminal domain. The paired domain and paired-type homeodomain each have DNA binding activities, while the P/S/T-rich domain possesses a transactivation function. Pax-6(5a) is a product of the alternatively spliced exon 5a resulting in a 14 residue insertion in the paired domain which alters the specificity of this DNA binding activity. The nucleotide sequence corresponding to the linker region encodes a set of three alternative translation start codons from which the third Pax-6 isoform originates. Collectively known as the Pax-6(ΔPD) or pairedless isoforms, these three gene products all lack a paired domain. The pairedless proteins possess molecular weights of 43, 33, or 32 kDa, depending on the particular start codon used. Pax-6 transactivation function is attributed to the variable length C-terminal P/S/T-rich domain which stretches to 153 residues in human and mouse proteins.

Of the four Drosophila Pax-6 orthologues, it is thought that the eyeless (ey) and twin of eyeless (toy) gene products share functional homology with the vertebrate canonical Pax-6 isoform, while the eyegone (eyg) and twin of eyegone (toe) gene products share functional homology with the vertebrate Pax-6(5a) isoform. Eyeless and eyegone were named for their respective mutant phenotypes.

### F. Cell Substrates

The disclosed methods involve incubation, culturing, growth and/or maintenance of cells. For this, various cell substrates can be used. For example, various culture flasks and plates can be used. Those of skill in the art are familiar with are variety of cell substrates and techniques for their use, which can be used or adapted for use with the disclosed methods.

The disclosed methods of producing neural cells can comprise, for example, different stages such as pre-incubation, incubations, culturing, and differentiation. Any suitable cell substrate can be used for each of these stages (as well as for other cell culture and growth steps or stages). However, in the disclosed methods, it is useful to use particular cell substrates for some methods. For example, it is useful to use a treated polymer substrate (such as CELLBIND™, or substrate treated with Matrigel™, Geltrex™, or fibronectin). Such cell substrates can aid in cell growth and maintenance in, for example, serum-free conditions and/or in the absence of feeder cells.

“Extracellular matrix” or “matrix” refers to one or more substances that provide substantially the same conditions for supporting cell growth as provided by an extracellular matrix synthesized by feeder cells. The matrix can be provided on a substrate, the component(s) comprising the matrix can be provided in solution, the matrix can be provided by a treated or altered substrate, or a combination.

Neuronal cell differentiation generally takes place in a culture environment comprising a suitable substrate and a nutrient medium containing differentiation agents are added. Suitable substrates include solid surfaces coated with a positive charge, such as a basic amino acid, exemplified by poly L-lysine and polyornithine. Substrates can be coated with extracellular matrix components, exemplified by fibronectin. Other permissive extracellular matrixes include Matrigel™ (extracellular matrix from Engelbreth-Holm-Swarm tumor cells), Geltrex™, laminin, and combination substrates, such as poly-L-lysine combined with fibronectin, laminin, or both. Extracellular matrix is useful, for example, when feeder cells are used. Any suitable extracellular matrix can be used with the disclosed methods and compositions.

Treated polymer substrate is useful, for example, when serum free conditions and/or no feeder cells are used. “Treated polymer substrate” refers to a polymer substrate that has been treated to make it more conducive to cell culture and growth. For example, the polymer substrate can be seeded or coated with useful molecules, or the polymer substrate can be manufactured or physically treated. Useful treated polymer substrates include those described in U.S. Patent Application Publication No. 20030180903 and/or treated by the method in U.S. Pat. No. 6,617,152 or U.S. Patent Application Publication No. 20080003663, each of which is specifically herein incorporated by reference for its description of polymer treated substrates and their use. A useful treated polymer substrate is CELLBIND™ (Corning). Matrigel™, Geltrex™, and fibronectin are examples of other coating substrates.

### G. Signaling Pathways

The disclosed methods can involve modulation of certain signaling pathways. Such signaling pathways have been discovered to be involved in neural induction and can be used to produce neural stem cells from pluripotent stem cells. For example, the disclosed methods can involve modulation of the phosphatidylinositol 3-kinase signaling pathway, the TGF-β superfamily signaling pathway, the Wnt signaling pathway, or a combination. For example, neural induction can be stimulated by activating the phosphatidylinositol 3-kinase signaling pathway in combination with inhibition of the TGF-β superfamily signaling pathway, inhibition of the Wnt signaling pathway, or a combination.

1. Phosphatidylinositol 3-kinase Signaling Pathway

Phosphatidylinositol 3-kinases (PI 3-kinases; PI3 Ks) have been linked to an extraordinarily diverse group of cellular functions, including cell growth, proliferation, differentiation, motility, survival and intracellular trafficking. Many of these functions relate to the ability of class I PI3Ks to activate protein kinase B (PKB, aka Akt). The class IA PI 3-kinase p110a is mutated in many cancers. Many of these mutations cause the kinase to be more active. The PtdIns(3,4,5)P₃ phosphatase PTEN which antagonizes PI 3-kinase signaling is absent from many tumors. Hence, PI 3-kinase activity contributes significantly to cellular transformation and the development of cancer. The p110δ and p110γ isoforms regulate different aspects of immune responses. PI 3-kinases are also a key component of the insulin signaling pathway.

AKT is activated as a result of PI3-kinase activity, because AKT requires the formation of the PtdIns(3,4,5)P3 (or “PIP3”) molecule in order to be translocated to the cell membrane. At PIP3, AKT is then phosphorylated by another kinase called phosphoinositide dependent kinase 1 (PDK1), and is thereby activated. The “PI3-k/AKT” signaling pathway has been shown to be required for an extremely diverse array of cellular activities—most notably cellular proliferation and survival. In addition to AKT and PDK1, one other related serine threonine kinase is bound at the PIP3 molecule created as a result of PI3-kinase activity, SGK.

Phosphatidylinositol 3-kinase has also been implicated in Long term potentiation (LTP). In mouse hippocampal CA1 neurons, PI3K is complexed with AMPA Receptors and compartmentalized at the postsynaptic density of glutamatergic synapses. PI3K is phosphorylated upon NMDA Receptor-dependent CaMKII activity, and it then facilitates the insertion of AMPA-R GluR1 subunits into the plasma membrane. This indicates that PI3K is required for the expression of LTP. Furthermore, PI3K inhibitors abolished the expression of LTP in rat hippocampal CAL but do not affect its induction. Notably, the dependence of late-phase LTP expression on PI3K seems to decrease over time. However, PI3K inhibitors suppressed the induction, but not the expression, of LTP in mouse hippocampal CA1. The PI3K pathway also recruits many other proteins downstream, including mTOR, GSK3β, and PSD-95. The PI3K-mTOR pathway leads to the phosphorylation of p70S6K, a kinase which facilitates translational activity, indicating that PI3K is required for the protein-synthesis phase of LTP induction instead. All PI 3-kinases are inhibited by the drugs wortmannin and LY294002, although certain member of the class II PI 3-kinase family show decreased sensitivity.

Cell signaling via 3′-phosphorylated phosphoinositides has been implicated in a variety of cellular processes, e.g., malignant transformation, growth factor signaling, inflammation, and immunity (see Rameh et al., J. Biol Chem, 274:8347-8350 (1999) for a review). The enzyme responsible for generating these phosphorylated signaling products, phosphatidylinositol 3-kinase (PI 3-kinase; PI3K), was originally identified as an activity associated with viral oncoproteins and growth factor receptor tyrosine kinases that phosphorylates phosphatidylinositol (PI) and its phosphorylated derivatives at the 3′-hydroxyl of the inositol ring (Panayotou et al., Trends Cell Biol 2:358-60 (1992)).

Evidence implicates PI3K and a lipid product of this enzyme, phosphatidylinositol (3,4,5)-triphosphate (“PtdIns(3,4,5)P₃”), as part of an important second messenger system in cellular signal transduction. The components of this PtdIns(3,4,5)P₃-based signaling system appear to be independent of the previously characterized signaling pathway based on inositol phospholipids, in which a phosphoinositidase C (PIC) hydrolyses PtdIns(4,5)P₂ to release the structurally distinct second messengers inositol (1,4,5)-triphosphate (Ins(1,4,5)P₃) and diacylglycerol.

The levels of phosphatidylinositol-3,4,5-triphosphate (PIP3), the primary product of PI 3-kinase activation, increase upon treatment of cells with a variety of agonists. PI 3-kinase activation, therefore, is involved in a range of cellular responses including cell growth, differentiation, and apoptosis (Parker et al., Current Biology, 5:577-99 (1995); Yao et al., Science, 267:2003-05 (1995)). Evidence indicates that pleckstrin-homology domain- and FYVE-finger domain-containing proteins are activated when binding to various phosphatidylinositol lipids (Sternmark et al., J Cell Sci, 112:4175-83 (1999); Lemmon et al., Trends Cell Biol, 7:237-42 (1997)). In vitro, some isoforms of protein kinase C (PKC) are directly activated by PIP3, and the PKC-related protein kinase, PKB, has been shown to be activated by PI 3-kinase (Burgering et al., Nature, 376:599-602 (1995)).

The PI 3-kinase enzyme family can been divided into three classes based on their substrate specificities. Class I PI3Ks can phosphorylate phosphatidylinositol (PI), phosphatidylinositol-4-phosphate, and phosphatidylinositol-4,5-biphosphate (PIP2) to produce phosphatidylinositol-3-phosphate (PIP), phosphatidylinositol-3,4-biphosphate, and phosphatidylinositol-3,4,5-triphosphate, respectively. Class II PI3Ks phosphorylate PI and phosphatidylinositol-4-phosphate, whereas Class III PI3Ks can only phosphorylate PI.

Furthermore, PI 3-kinase appears to be involved in a number of aspects of leukocyte activation. A p85-associated PI 3-kinase activity has been shown to physically associate with the cytoplasmic domain of CD28, which is an important co-stimulatory molecule for the activation of T-cells in response to antigen (Pages et al., Nature, 369:327-29 (1994); Rudd, Immunity, 4:527-34 (1996)). Activation of T cells through CD28 lowers the threshold for activation by antigen and increases the magnitude and duration of the proliferative response. These effects are linked to increases in the transcription of a number of genes including interleukin-2 (IL2), an important T cell growth factor (Fraser et al., Science, 251:313-16 (1991)). Mutation of CD28 such that it can no longer interact with PI 3-kinase leads to a failure to initiate IL2 production, suggesting a critical role for PI 3-kinase in T cell activation.

PI3K enzymes interact directly with, and may be co-purified with, activated forms of several receptor tyrosine kinases. Receptor tyrosine kinase associated PI3K consists of 170-200 kD heterodimers (Otsu et al., 1991, Cell 65:91-104, Pons et al., 1995, Mol. Cell. Biol. 15:4453-4465, Inukai et al., 1996, J. Biol. Chem. 271:5317-5320) comprising a catalytic subunit and an adapter (or regulatory) subunit.

PI 3-kinase is a heterodimer consisting of p85 and p110 subunits (Otsu et al., Cell, 65:91-104 (1991); Hiles et al., Cell, 70:419-29 (1992)). Four distinct Class I PI3Ks have been identified, designated PI3K α, β, δ, and γ, each consisting of a distinct 110 kDa catalytic subunit and a regulatory subunit. More specifically, three of the catalytic subunits, i.e., p110α, p110β and p110δ, each interact with the same regulatory subunit, p85; whereas p110γ interacts with a distinct regulatory subunit, p101.

2. MAPK Signaling Pathway

The mitogen-activated protein kinase (MAPK) cascade is a major signaling system that is shared by various types of cells. They are serine/threonine kinases. They can include MAPK1 1, MAPK1 4, MAPK12, and MAPK13. The mitogen-activated protein kinase 11 (MAPK1 1; also known as stress-activated protein kinase 2, SAPK2, SAPK2B, p38B, p38-2, p38Beta, P38BETA2, and PRKM1 1), mitogen-activated protein kinase 12 (MAPK1 2; also known as stress-activated protein kinase 3, SAPK3, SAPK-3, mitogen-activated protein kinase 3, ERK3, extracellular signal-regulated kinase 6, ERK6, P38GAMMA, p38gamma, and PRKM 12), mitogen-activated protein kinase 13 (MAPK13; also known as stress-activated protein kinase 4, SAPK4, MGC99536, p38delta, and PRKM13), and mitogen-activated protein kinase 14 (MAPK14; also known as stress-activated protein kinase 2A, SAPK2A, p38alpha p38, cytokine suppressive antiinflammatory drug binding protein, CSBP, CSAID-binding protein, CSBP1, CSBP2, CSPB1, MAX-interacting protein 2, EXIP, RK, Mxi2, PRKM 14, and PRKM 15). They translocate on activation (phosphorylation) into nucleus. More detail regarding MAPK1 1, MAPK12, MAPK13, and MAPK14 are described at the website ncbi.nlm nih.gov/entrez/query.fcgi?db=OMIM, which is in the Online Mendelian Inheritance in Man database (OMIM Accession No. 602898, 602399, 602899, 600289, respectively).

The MAPK signaling pathway phosphorylates/activates many different proteins including transcription factors including those that regulate expression of important cell-cycle and differentiation specific proteins. The genes regulated generally are involved in apoptosis, inflammation, cell growth, and differentiation. The proteins mediate varieties of cellular responses and biological activities including morphogenesis, cell death, stress responses, immune responses, cell proliferation, apoptosis, paraapoptosis, cell survival etc.

Activation of MAPK cascade is not restricted to immature cells, and this cascade is also activated in terminally differentiated cells such as neutrophils, indicating that the MAPK cascade also plays an important role in some functions of terminally differentiated mature cells. Activation of MAP kinase in two different cells can lead to similar or different cellular responses. The ERK cascade is activated in response to signals from receptor tyrosine kinases, hematopoietic growth factor receptors, or some heterotrimeric G-protein-coupled receptors and appears to mediate signals promoting cell proliferation or differentiation.

The stress activated protein kinase (SAPK), includes p38 and JNk, is activated in response to heat shock, hyperosmolarity, UV irradiation, protein synthesis inhibitors or inflammatory cytokines and appear to be involved in the cell responses to stresses. Activation of the distinct MAPK subtype cascade is dependent on the types of cells and the stimuli used. The functional role of each MAPK subtype may be different according to the types of cells.

ERK2 is a widely distributed protein kinase that achieves maximum activity when both Thr183 and Tyr185 are phosphorylated by the upstream MAP kinase kinase, MEK1 (Anderson et al., Nature, 1990, 343, 651; Crews et al., Science 1992, 258, 478). Upon activation, ERK2 phosphorylates many regulatory proteins, including the protein kinases Rsk90 (Bjorbaek et al., J. Biol. Chem. 1995, 270; 18848) and MAPKAP2 (Rouse et al., Cell 1994, 78; 1027), and transcription factors such as ATF2 (Raingeaud et al., Mol. Cell Biol. 1996, 16; 1247), Elk-1 (Raingeaud et al. Mol. Cell Biol. 1996, 16; 1247), c-Fos (Chen et al., Proc. Natl. Acad. Sci. USA 1993, 90; 10952), and c-Myc (Oliver et al., Proc. Soc. Exp. Biol. Med. 1995, 210; 162). ERK2 is also a downstream target of the Ras/Raf dependent pathways (Moodie et al., Science 1993, 260; 1658) and may help relay the signals from these potentially oncogenic proteins. ERK2 has been shown to play a role in the negative growth control of breast cancer cells (Frey and Mulder, Cancer Res. 1997, 57; 628) and hyperexpression of ERK2 in human breast cancer has been reported (Sivaraman et al., “J. Clin. Invest. 1997, 99; 1478). Activated ERK2 has also been implicated in the proliferation of endothelin-stimulated airway smooth muscle cells, suggesting a role for this kinase in asthma (Whelchel et al., Am. J. Respir. Cell Mol. Biol. 1997, 16; 589).

U.S. Pat. No. 6,994,981 describe modulators of para-apoptosis and related methods. EP1208748, WO 2004089929 & WO2006117567 are prior art patents based on MAPK inhibitors. U.S. Pat. No. 6,852,740 B2 describe pyrazole derivatives as p38 kinase inhibitors. WO 95/31451 describes pyrazole compositions that inhibit MAPKs, and, in particular, p38.

The part of the MAPK cascade most relevant herein is the part activated by Midkine and insulin-like growth factor-1.

3. TGF-β Superfamily Signaling Pathway

The transforming growth factor beta (TGF-β) superfamily is a large family of structurally related cell regulatory proteins that was named after its first member, TGF-β1, originally described in 1983 (Assoian R, et al. J Biol Chem 258 (11): 7155-60). Many growth factors from the TGF-β superfamily (Kingsley, Genes and Development 8, 133-146 (1994)) are relevant for a wide range of medical treatment methods and applications which in particular concern promotion of cell proliferation and tissue formation, including wound healing and tissue reproduction. Such growth factors in particular comprise members of the TGF-β (transforming growth factor; Roberts and Sporn, Handbook of Experimental Pharmacology 95 (1990), page 419-472, editors: Sporn and Roberts), the DVR-group (Hotten et al., Biochem. Biophys. Res. Comm 206 (1995), page 608-613 and further literature cited therein) including BMPs (bone morphogenetic protein; Rosen and Thies, Growth Factors in Perinatal Development (1993), page 39-58, editors: Tsang, Lemons and Balistreri) and GDFs (growth differentiation factors), the inhibin/activin (Vale et al., The Physiology of Reproduction, second edition (1994), page 1861-1878, editors: Knobil and Neill) and the GDNF protein family (Rosenthal, Neuron 22 (1999), page 201-203; Airaksinen et al. Mol Cell Neurosci 13 (1999), page 313-325).

Although the members of the TGF-β superfamily show high amino acid homologies in the mature part of the protein, in particular 7 conserved cysteines, they show considerable variations in their exact functions. Often individual growth factors of these families exhibit a plurality of functions at the same time, so that their application is of interest in various medical indications. Some of these multifunctional proteins also have survival promoting effects on neurons in addition to functions such as e.g. regulation of the proliferation and differentiation in many cell types (Roberts and Sporn; Sakurai et al., J. Biol. Chem. 269 (1994), page 14118-14122). Thus, for example, trophic effects on embryonic motoric and sensory neurons were demonstrated for TGF-β in vitro (Martinou et al., Devl. Brain Res. 52, page 175-181 (1990) and Chalazonitis et al., Dev. Biol. 152, page 121-132 (1992)). In addition, effects promoting survival are shown for dopaminergic neurons of the mid-brain for the proteins TGF-0-1, -2, -3, activin A and GDNF (glial cell line-derived neurotrophic factor), a protein which has structural similarities to TGF-β superfamily members, these effects being not mediated via astrocytes (Krieglstein et al., EMBO J. 14, page 736-742 (1995)).

The occurrence of proteins of the TGF-β superfamily in various tissuous stages and development stages corresponds with differences with regard to their exact functions as well as target sites, life span, requirements for auxiliary factors, necessary cellular physiological environment and/or resistance to degradation.

The proteins of the TGF-β superfamily exist as homodimers or heterodimers having a single disulfide bond. This disulfide bond is mediated by a specific and in most of the proteins conserved cysteine residue of the respective monomers. Interesting members of the TGF-β superfamily or active variants thereof comprise the TGF-β proteins like TGF-β1, TGF-β2, TGF-3, TGF-β4, TGF-β5 (U.S. Pat. No. 5,284,763; EP 0376785; U.S. Pat. No. 4,886,747; DNA 7 (1988), page 1-8), EMBO J. 7 (1988), page 3737-3743), Mol. Endo. 2 (1988), page 1186-1195), J. Biol. Chem. 265 (1990), page 1089-1093), OP1, OP2 and OP3 proteins (U.S. Pat. No. 5,011,691, U.S. Pat. No. 5,652,337, WO 91/05802) as well as BMP2, BMP3, BMP4 (WO 88/00205, U.S. Pat. No. 5,013,649 and WO 89/10409, Science 242 (1988), page 1528-1534), BMP5, BMP6 and BMP-7 (OP1) (Proc. Natl. Acad. Sci. 87 (1990), page 9841-9847, WO 90/11366), BMP8 (OP2) (WO 91/18098), BMP9 (WO 93/00432), BMP10 (WO 94/26893), BMP11 (WO 94/26892), BMP12 (WO 98/16035), BMP13 (WO 95/16035), BMP15 (WO 96/36710), BMP16 (WO 98/12322), BMP3b (Biochem. Biophys. Res. Comm 219 (1996), page 656-662), GDF1 (WO 92/00382 and Proc. Natl. Acad. Sci. 88 (1991), page 4250-4254), GDF8 (WO 94/21681), GDF10 (WO95/10539), GDF11 (WO 96/01845), GDF5 (CDMP1, MP52) (WO 95/04819; WO96/01316; WO 94/15949, WO 96/14335 and WO 93/16099 and Nature 368 (1994), page 639-643), GDF6 (CDMP2, BMP13) (WO 95/01801, WO 96/14335 and WO95/16035), GDF7 (CDMP3, BMP12) (WO 95/01802 and WO 95/10635), GDF14 (WO 97/36926), GFD15 (WO 99/06445), GDF16 (WO 99/06556), 60A (Proc. Natl. Acad. Sci. 88 (1991), page 9214-9218), DPP (Nature 325 (1987), page 81-84), Vgr-1 (Proc. Natl. Acad. Sci. 86 (1989), page 4554-4558) Vg-1, (Cell 51 (1987), page 861-867), dorsalin (Cell 73 (1993), page 687-702), MIS (Cell 45 (1986), page 685-698), pCL13 (WO 97/00958), BIP (WO 94/01557), inhibin a, activin βA and activin βB (EP 0222491), activin βC (MP121) (WO 96/01316), activin βE and GDF12 (WO 96/02559 and WO 98/22492), activin βD (Biochem. Biophys. Res. Comm 210 (1995), page 581-588), GDNF (Science 260 (1993), page 1130-1132, WO 93/06116), Neurturin (Nature 384 (1996), page 467-470), Persephin (Neuron 20 (1998), page 245-253, WO 97/33911), Artemin (Neuron 21 (1998), page 1291-1302), Mic-1 (Proc. Natl. Acad. Sci USA 94 (1997), page 11514-11519), Univin (Dev. Biol. 166 (1994), page 149-158), ADMP (Development 121 (1995), page 4293-4301), Nodal (Nature 361 (1993), page 543-547), Screw (Genes Dev. 8 (1994), page 2588-2601). Other useful proteins include biologically active biosynthetic constructs including biosynthetic proteins designed using sequences from two or more known morphogenetic proteins. Examples of biosynthetic constructs are disclosed in U.S. Pat. No. 5,011,691 (e.g. COP-1, COP-3, COP-4, COP-5, COP-7 and COP-16).

Most of the members of the TGF-β protein superfamily are morphogenetic proteins that are useful for treatments where regulation of differentiation and proliferation of cells or progenitor cells is of interest. This can result in replacement of damaged and/or diseased tissue like for example skeletal (bone, cartilage) tissue, connective tissue, periodontal or dental tissue, neural tissue, tissue of the sensory system, liver, pancreas, cardiac, blood vessel and renal tissue, uterine or thyroid tissue etc. Morphogenetic proteins are often useful for the treatment of ulcerative or inflammatory tissue damage and wound healing of any kind such as enhanced healing of ulcers, burns, injuries or skin grafts. Several BMP proteins which were originally discovered by their ability to induce bone formation, have been described, as also indicated above. Meanwhile, several additional functions have been found as it is also true for members of the GDFs. These proteins show a very broad field of applications and especially are in addition to their bone and cartilage growth promoting activity (see for example: WO 88/00205, WO 90/11366, WO 91/05802) useful in periodontal disease, for inhibiting periodontal and tooth tissue loss, for sealing tooth cavities, for enhancing integration of a tooth in a tooth socket (see for example: WO 96/26737, WO 94/06399, WO 95/24210), for connective tissue such as tendon or ligament (see for example: WO 95/16035), for improving survival of neural cells, for inducing growth of neural cells and repairing neural defects, for damaged CNS tissue due to stroke or trauma (see for example: WO 97/34626, WO 94/03200, WO 95/05846), for maintaining or restoring sensory perception (see for example WO 98/20890, WO 98/20889), for renal failure (see for example: WO 97/41880, WO 97/41881), for liver regeneration (see for example WO 94/06449), for regeneration of myocardium (see for example WO 98/27995), for treatment or preservation of tissues or cells for organ or tissue transplantation, for integrity of gastrointestinal lining (see for example WO 94/06420), for increasing progenitor cell population as for example hematopoietic progenitor cells by ex vivo stimulation (see for example WO 92/15323).

4. Wnt Signaling Pathway

Wnt genes encode a large family of secreted, cystein rich proteins that play key roles as intercellular signaling molecules in a wide variety of biological processes. The first Wnt gene, mouse wnt-1, was discovered as a proto-oncogene activated by integration of mouse mammary tumor virus in mammary tumors (Nusse and Varmus 1982). Consequently, the involvement of the Wnt pathway in cancer has been largely studied. Mutational analysis in mice has shown the importance of Wnts in controlling diverse developmental processes such as patterning of the body axis, central nervous system and limbs, and the regulation of inductive events during organogenesis. The Wnt family of secreted growth factors initiates signaling via the Frizzled (Fz) receptor and its coreceptor, LDL receptor-related protein 5 or 6 (LPR5 or LRP6), presumably through Fz-LPR5/LRP6 complex formation induced by Wnt.

Through the identification of the Drosophila polarity gene wingless (wg) as a wnt-1 homologue (Cabrera, Alonso et al. 1987; Perrimon and Mahowald 1987; Rijsewijk, Schuermann et al. 1987), it became clear that wnt genes are important developmental regulators. Thus, although at first glance dissimilar, biological processes like embryogenesis and carcinogenesis both rely on cell communication via identical signaling pathways. In a current model of the pathway, the secreted Wnt protein binds to Frizzle cell surface receptors and activates the cytoplasmic protein Dishevelled (Dsh). Dsh then transmits the signal to a complex of several proteins, including the protein kinase Shaggy/GSK3 (Sgg), the APC tumor supressor, the scaffold protein Axin and β-Catenin (β-Cat), the vertebrate homologue of Drosophila Armadillo. In this complex β-Cat is targeted for degradation after being phosphorylated by Sgg. After Wnt signaling and the resulting down-regulation of Sgg activity, β-Cat (or its Drosophila homologue Armadillo) escape from degradation and accumulate into the cytoplasm. Free cytoplasmic β-Cat translocates to the nucleus by a still obscure mechanism, and modulates gene transcription through binding the Tcf/Lef family of transcription factors (Grosschedl R 1999). Mutations of β-Cat itself or of negative regulatory elements, like APC and Axin, that lead to nuclear accumulation of β-Cat and consequently to constitutive activation of the Wnt pathway have been observed in many types of cancers, including colon, skin and breast cancer (Barker N 1999; Morin 1999; Potter 1999; Roose and Clevers 1999; Waltzer and Bienz 1999).

Wnt signaling pathway regulates a variety of processes including cell growth, oncogenesis, and development (Moon et al., 1997, Trends Genet. 13, 157-162: Miller et al., 1999, Oncogene 18, 7860-7872: Nusse and Varmus, 1992, Cell 69, 1073-1087: Cadigan and Nusse, 1997, Genes Dev. 11, 3286-3305: Peifer and Polakis, 2000 Science 287, 1606-1609: Polakis 2000, Genes Dev. 14, 1837-1851). Wnt signaling pathway has been intensely studied in a variety of organisms. The activation of TCF4/β-catenin mediated transcription by Wnt signal transduction has been found to play a key role in its biological functions (Molenaar et al., 1996, Cell 86, 391-399: Gat et al., 1998 Cell 95, 605-614: Orford et al., 1999 J. Cell. Biol. 146, 855-868).

In the absence of Wnt signals, tumor suppressor gene adenomatous polyposis coli (APC) simultaneously interact with the serine kinase glycogen synthase kinase (GSK)-3β and β-catenin (Su et al., 1993, Science 262, 1734-1737: Yost et al., 1996 Genes Dev. 10, 1443-1454: Hayashi et al., 1997, Proc. Natl. Acad. Sci. USA, 94, 242-247: Sakanaka et al., 1998, Proc. Natl. Acad. Sci. USA, 95, 3020-3023: Sakanaka and William, 1999, J. Biol. Chem 274, 14090-14093). Phosphorylation of APC by GSK-3β regulates the interaction of APC with β-catenin, which in turn may regulate the signaling function of β-catenin (B. Rubinfeld et al., Science 272, 1023, 1996). Wnt signaling stabilizes β-catenin allowing its translocation to the nucleus where it interacts with members of the lymphoid enhancer factor (LEF1)/T-cell factor (TCF4) family of transcription factors (Behrens et al., 1996 Nature 382, 638-642: Hsu et al., 1998, Mol. Cell. Biol. 18, 4807-4818: Roose et all., 1999 Science 285, 1923-1926).

Moreover, overexpression of several downstream mediators of Wnt signaling has been found to regulate apoptosis (Moris et al., 1996, Proc. Natl. Acad. Sci. USA, 93, 7950-7954: He et al., 1999, Cell 99, 335-345: Orford et al., 1999 J. Cell. Biol., 146, 855-868: Strovel and Sussman, 1999, Exp. Cell. Res., 253, 637-648). Overexpression of APC in human colorectal cancer cells induced apoptosis (Moris et al., 1996, Proc. Natl. Acad. Sci. USA., 93, 7950-7954), ectopic expression of β-catenin inhibited apoptosis associated with loss of attachment to extracellular matrix (Orford et al, 1999, J. Cell Biol. 146, 855-868). Inhibition of TCF4/β-catenin transcription by expression of dominant-negative mutant of TCF4 blocked Wnt-1-mediated cell survival and rendered cells sensitive to apoptotic stimuli such as anti-cancer agent (Shaoqiong Chen et al., 2001, J. Cell. Biol., 152, 1, 87-96) and APC mutation inhibits apoptosis by allowing constitutive survivin expression, a well-known anti-apoptotic protein (Tao Zhang et al., 2001, Cancer Research, 62, 8664-8667).

There are various ways to specifically inhibit Wnt signaling can be inhibited in a variety of ways. For example, the (secreted) Wnt signal can be blocked by an excess of the ligand binding domain of its receptor, Frizzled. This domain is best made as its natural fusion in the FRP/Frzb form. Alternatively, it can be expressed on the surface of target cells using a GPI anchor, which works well (Cadigan, 1998). Another way of inhibiting Wnt is to add excess of Dickkopf (Dkk) protein (Glinka, 1998). This works well in cell culture and in vivo. Dkk binds to the LRP co-receptor for Wnt. To block signaling inside cells, several workers have used dominant negative Dishevelled (Wallingford 2000). Overexpressing intact Axin, a negative regulator of the Wnt pathway, works very well (Zeng, 1997, Itoh 1998; Willert, 1999). Overexpressing full length GSK can also block Wnt signaling effectively (He, 1995). There are dominant negative forms of TCF that can be used to block Wnt signaling in the nucleus (Molenaar 1996). RNAi targeting of various component of the pathway could be used, such as LRP/Arrow, Dishevelled, This has been shown to work very well for Drosophila S2 cells (Matsubayashi 2004, Cong et al, 2004) but also in mammalian cells (Lu et al, 2004).

Secreted frizzled related protein 2 (Sfrp2) is known to modulate Wnt signaling, and cells treated with secreted frizzled related protein increase cellular beta-catenin and up-regulate expression of antiapoptotic genes. Frzb is a family of secreted glycoproteins that function to modulate signaling activity of Wnt. Frzb proteins share sequence homology with the extracellular domain of the Wnt receptor (frizzled) and are capable of binding to Wnt. Thus, Frzb functions to antagonize Wnt activity by sequestering Wnt and preventing its binding to the frizzled receptor. Two human frzb homologues of Frzb are termed hFRP-1b and hFRP-2.

Pax-6 regulates expression of SFRP-2 and Wnt-7b in the developing CNS. Wnt signaling regulates a wide range of developmental processes such as proliferation, cell migration, axon guidance, and cell fate determination. Secreted frizzled related protein-2 (SFRP-2) is expressed in several discrete neuroepithelial domains, including the diencephalon, the insertion of the eminentia thalami into the caudal telencephalon, and the pallial-subpallial boundary (PSB). Wnt-7b expression is similar to SFRP-2 expression. The head inducer Cerberus is a multifunctional antagonist of Nodal, BMP and Wnt signals.

5. Notch Signaling Pathway

Maturation of the Notch receptor involves cleavage at the prospective extracellular side during intracellular trafficking in the Golgi complex. This results in a bipartite protein, composed of a large extracellular domain linked to the smaller transmembrane and intracellular domain. Binding of ligand promotes two proteolytic processing events; as a result of proteolysis, the intracellular domain is liberated and can enter the nucleus to engage other DNA-binding proteins and regulate gene expression.

Notch and most of its ligands are transmembrane proteins, so the cells expressing the ligands typically must be adjacent to the Notch expressing cell for signaling to occur. The Notch ligands are also single-pass transmembrane proteins and are members of the DSL (Delta/Serrate/LAG-2) family of proteins. In Drosophila melanogaster there are two ligands named Delta and Serrate. In mammals, the corresponding names are Delta-like and Jagged. In mammals there are multiple Delta-like and Jagged ligands, as well as a variety of other ligands, such as F3/contactin. There has been at least one report that suggests that some cells can send out processes that allow signaling to occur between cells that are as much as four or five cell diameters apart.

The Notch extracellular domain is composed primarily of small cysteine knot motifs called EGF-like repeats. Notch 1, for example, has 36 of these repeats. Each EGF-like repeat is comprised of approximately 40 amino acids, and its structure is defined largely by six conserved cysteine residues that form three conserved disulfide bonds. Each EGF-like repeat can be modified by O-linked glycans at specific sites. An O-glucose sugar may be added between the first and second conserved cysteines, and an O-fucose may be added between the second and third conserved cysteines. These sugars are added by an as-yet-unidentified O-glucosyltransferase, and GDP-fucose Protein O-fucosyltransferase 1 (POFUT1), respectively. The addition of O-fucose by POFUT1 is absolutely necessary for Notch function, and, without the enzyme to add O-fucose, all Notch proteins fail to function properly.

The O-glucose on Notch can be further elongated to a trisaccharide with the addition of two xylose sugars by xylosyltransferases, and the O-fucose can be elongated to a tetrasaccharide by the ordered addition of an N-acetylglucosamine (GlcNAc) sugar by an N-Acetylglucosaminyltransferase called Fringe, the addition of a galactose by a galactosyltransferase, and the addition of a sialic acid by a sialyltransferase.

In mammals there are three Fringe GlcNAc-transferases, named Lunatic Fringe, Manic Fringe, and Radical Fringe. These enzymes are responsible for something called a “Fringe Effect” on Notch signaling. If Fringe adds a GlcNAc to the O-fucose sugar, then the subsequent addition of a galactose and sialic acid will occur. In the presence of this tetrasaccharide, Notch signals strongly when it interacts with the Delta ligand, but has markedly inhibited signaling when interacting with the Jagged ligand.

Once the Notch extracellular domain interacts with a ligand, an ADAM-family metalloprotease called TACE (Tumor Necrosis Factor Alpha Converting Enzyme) cleaves the Notch protein just outside the membrane. This releases the extracellular portion of Notch, which continues to interact with the ligand. The ligand plus the Notch extracellular domain is then endocytosed by the ligand-expressing cell. There may be signaling effects in the ligand-expressing cell after endocytosis. After this first cleavage, an enzyme called γ-secretase cleaves the remaining part of the Notch protein just inside the inner leaflet of the cell membrane of the Notch-expressing cell. This releases the intracellular domain of the Notch protein, which then moves to the nucleus, where it can regulate gene expression by activating the transcription factor CSL. Other proteins also participate in the intracellular portion of the Notch signaling cascade.

6. Protein Kinase A Signaling Pathway

Protein kinase A (PKA) is a family of enzymes whose activity is dependent on the level of cyclic AMP (cAMP) in the cell. PKA is also known as cAMP-dependent protein kinase. Protein kinase A has several functions in the cell, including regulation of glycogen, sugar, and lipid metabolism.

Each PKA is a holoenzyme that consists of two regulatory and two catalytic subunits. Under low levels of cAMP, the holoenzyme remains intact and is catalytically inactive. When the concentration of cAMP rises (e.g., activation of adenylate cyclases by G protein-coupled receptors coupled to Gₛ, inhibition of phosphodiesterases that degrade cAMP), cAMP binds to the two binding sites on the regulatory subunits, which leads to the release of the catalytic subunits. The free catalytic subunits can then catalyze the transfer of ATP terminal phosphates to protein substrates at serine, or threonine residues. This phosphorylation usually results in a change in activity of the substrate. Since PKAs are present in a variety of cells and act on different substrates, PKA and cAMP regulation are involved in many different pathways.

Protein kinase is bound by cAMP, which is thought to have a role in the control of cell proliferation and differentiation (see, e.g., Cho-Chung (1980) J. Cyclic Nucleotide Res. 6:163 167) There are two types of PKA, type I (PKA-I) and type II (PKA-II), both of which share a common C subunit but each containing distinct R subunits, RI and Rh, respectively (Beebe et al. in The Enzymes: Control by Phosphorylation, 17(A):43 111 (Academic, New York, 1986). The R subunit isoforms differ in tissue distribution (Oyen et al. (1988) FEBS Lett. 229:391 394; Clegg et al. (1988) Proc. Natl. Acad. Sci. (USA) 85:3703 3707) and in biochemical properties (Beebe et al. in The Enzymes: Control by Phosphorylation, 17(A): 43 111 (Academic Press, NY, 1986); Cadd et al. (1990) J. Biol. Chem. 265:19502 19506). The two general isoforms of the R subunit also differ in their subcellular localization: RI is found throughout the cytoplasm; whereas RII localizes to nuclei, nucleoli, Golgi apparatus and the microtubule-organizing center (see, e.g., Lohmann in Advances in Cyclic Nucleotide and Protein Phosphorylation Research, 18: 63 117 (Raven, N.Y., 1984; and Nigg et al. (1985) Cell 41:1039 1051).

An increase in the level of RI_{α} expression has been demonstrated in human cancer cell lines and in primary tumors, as compared with normal counterparts, in cells after transformation with the Ki-ras oncogene or transforming growth factor-α, and upon stimulation of cell growth with granulocyte-macrophage colony-stimulating factor (GM-CSF) or phorbol esters (Lohmann in Advances in Cyclic Nucleotide and Protein Phosphorylation Research, 18:63 117 (Raven, N.Y., 1984); and Cho-Chung (1990) Cancer Res. 50:7093 7100). Conversely, a decrease in the expression of RIₐ has been correlated with growth inhibition induced by site-selective cAMP analogs in a broad spectrum of human cancer cell lines (Cho-Chung (1990) Cancer Res. 50:7093 7100). It has also been determined that the expression of RI/PKA-I and RII/PKA-II has an inverse relationship during ontogenic development and cell differentiation (Lohmann in Advances in Cyclic Nucleotide and Protein Phosphorylation Research, Vol. 18, 63 117 (Raven, N.Y., 1984); Cho-Chung (1990) Cancer Res. 50:7093 7100). The RIₐ subunit of PKA has thus been hypothesized to be an ontogenic growth-inducing protein whose constitutive expression disrupts normal ontogenic processes, resulting in a pathogenic outgrowth, such as malignancy (Nesterova et al. (1995) Nature Medicine 1:528 533).

### H. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for detecting the state or condition of a cell, the kit comprising, for example, neural cells produced by the disclosed methods. As another example, disclosed are kits comprising NDF for induction of neural stem cells. The kits also can contain labels and other reagents for detection of biomolecules.

### I. Mixtures

Disclosed are mixtures formed by performing or preparing to perform the disclosed method. For example, disclosed are mixtures comprising NDF and stem cells.

Whenever the method involves mixing or bringing into contact compositions or components or reagents, performing the method creates a number of different mixtures. For example, if the method includes 3 mixing steps, after each one of these steps a unique mixture is formed if the steps are performed separately. In addition, a mixture is formed at the completion of all of the steps regardless of how the steps were performed. The present disclosure contemplates these mixtures, obtained by the performance of the disclosed methods as well as mixtures containing any disclosed reagent, composition, or component, for example, disclosed herein.

### J. Systems

Disclosed are systems useful for performing, or aiding in the performance of, the disclosed method. Systems generally comprise combinations of articles of manufacture such as structures, machines, devices, and the like, and compositions, compounds, materials, and the like. Such combinations that are disclosed or that are apparent from the disclosure are contemplated. For example, disclosed and contemplated are systems comprising a cell growth apparatus and the disclosed stem cells and/or disclosed neural cells.

### K. Data Structures and Computer Control

Disclosed are data structures used in, generated by, or generated from, the disclosed method. Data structures generally are any form of data, information, and/or objects collected, organized, stored, and/or embodied in a composition or medium. A cell growth protocol stored in electronic form, such as in RAM or on a storage disk, is a type of data structure.

The disclosed method, or any part thereof or preparation therefor, can be controlled, managed, or otherwise assisted by computer control. Such computer control can be accomplished by a computer controlled process or method, can use and/or generate data structures, and can use a computer program. Such computer control, computer controlled processes, data structures, and computer programs are contemplated and should be understood to be disclosed herein.

### Uses

The disclosed methods and compositions are applicable to numerous areas including, but not limited to, production of neural cell, neural stem cell, and differentiated neural cell. Other uses include treatment of subjects using the disclosed neural cell, neural stem cell, and differentiated neural cell. The neural stem cells can also be used as research tools. Other uses are disclosed, apparent from the disclosure, and/or will be understood by those in the art.

### Methods

Disclosed are methods for producing neural cells from stem cells. For example, the disclosed methods involve generation of neural stem cells from pluripotent stem cells. Also disclosed are methods of using neural cells and neural stem cells produced in the disclosed methods. Also disclosed are methods of treating a subject using the neural cells and neural stem cells produced in the disclosed methods. Also disclosed are methods of treating a subject using the neural cells and neural stem cells produced in the disclosed methods where the neural cells were produced from pluripontent stem cells derived from the subject. Also disclosed are methods of detecting a state or characteristic of neural cells and neural stem cells produced in the disclosed methods. Also disclosed are methods testing conditions for differentiation of neural stem cells using the neural cells and neural stem cells produced in the disclosed methods.

For example, disclosed are methods of producing neural cells, the method comprising incubating pluripotent stem cells in the presence of an amount of one or more Neural Development Factors (NDF) sufficient to generate cells expressing Pax-6, Otx2, Nestin, or a combination, thereby producing neural cells. Also disclosed are neural cells produced by one or more of the disclosed methods. Also disclosed are neural cells produced by the method comprising incubating pluripotent stem cells in the presence of an amount of one or more Neural Development Factors (NDF) sufficient to generate cells expressing Pax-6, Otx2, Nestin, or a combination.

Also disclosed are methods of treating a subject, the method comprising administering a neural cell produced by one or more of the disclosed methods. Also disclosed are methods of treating a subject, the method comprising incubating pluripotent stem cells in the presence of an amount of one or more Neural Development Factors (NDF) sufficient to generate cells expressing Pax-6, Otx2, Nestin, or a combination, thereby producing neural cells, and administering one or more of the neural cells to the subject. The stem cell can be, for example, from the same species as the subject. The stem cell can be, for example, from the subject.

Also disclosed are methods of detecting a state or characteristic of a cell, the method comprising detecting the state or characteristic in a neural cell produced by one or more of the disclosed methods. Also disclosed are methods of testing conditions for differentiation of neural stem cells, the method comprising exposing a neural cell produced by one or more of the disclosed methods to test conditions and determining if the neural stem cells differentiate into a cell type of interest. The cell type of interest can be, for example, neuron, astrocyte, oligodendrocyte, or a combination.

Also disclosed are methods of producing neural cells, the method comprising incubating pluripotent stem cells in the presence of an amount of one or more Neural Development Factors (NDF) sufficient to generate cells expressing Pax-6, Otx2, Nestin, or a combination, thereby producing neural cells, wherein the NDF activate the phosphatidylinositol 3-kinase signaling pathway, inhibit the TGF-β superfamily signaling pathway, and inhibit the Wnt signaling pathway, wherein the NDF comprise Midkine, Pleiotrophin, insulin-like growth factor-1, or a combination; A83-01, SB431542, or a combination; dorsopmorphin; and PNU-74654, Dickkopf, or a combination.

Also disclosed are methods of producing neural cells, the method comprising (a) culturing pluripotent stem cells in the presence of fibroblast growth factor 2 (FGF-2); (b) incubating the stem cells in the presence of an amount of one or more Neural Development Factors (NDF) sufficient to generate cells expressing Pax-6, Otx2, Nestin, or a combination, thereby producing neural cells, wherein the NDF activate the phosphatidylinositol 3-kinase signaling pathway, inhibit the TGF-β superfamily signaling pathway, and inhibit the Wnt signaling pathway, wherein the NDF comprise Midkine, Pleiotrophin, insulin-like growth factor-1, or a combination; A83-01, SB431542, or a combination; dorsopmorphin; and PNU-74654, Dickkopf, or a combination, wherein prior to incubating in the presence of the NDF, at the same time as incubating in the presence of the NDF is initiated, or during incubating in the presence the NDF the use of FGF-2 is discontinued; and (c) culturing the neural cells, wherein the neural cells are cultured on a treated polymer substrate, wherein the neural cells are cultured in serum free conditions, wherein the neural cells are cultured in the presence of fibroblast growth factor 2 (FGF-2). The treated polymer substrate can be, for example, CELLBIND™, or substrate treated with Matrigel™, Geltrex™, or fibronectin. The treated polymer substrate can comprise, for example, CELLBIND™, or substrate treated with Matrigel™, Geltrex™, or fibronectin. The neural cells can be cultured in the presence of epidermal growth factor (EGF). The neural cells can be cryopreservable. The stem can also be cultured in the presence of conditioned media or chemically defined media such as mouse embryonic fibroblast-conditioned media (MEF-CM), mTeSR™ or StemPro™. The stem cells can also be incubated in the presence of 20% knockout serum replacement (KSR). Use of conditioned media can be discontinued when the use of FGF-2 is discontinued.

Also disclosed are methods of producing neural cells, the method comprising incubating pluripotent stem cells in the presence of an amount of one or more Neural Development Factors (NDF) sufficient to generate cells expressing Pax-6, Otx2, Nestin, or a combination, thereby producing neural cells, wherein the NDF activate the phosphatidylinositol 3-kinase signaling pathway, inhibit the TGF-β superfamily signaling pathway, and inhibit the Wnt signaling pathway, wherein the NDF comprise Midkine, Pleiotrophin, insulin-like growth factor-1, or a combination; A83-01, SB431542, or a combination; dorsopmorphin; and PNU-74654, Dickkopf, or a combination.

Also disclosed are methods of producing neural cells, the method comprising (a) culturing pluripotent stem cells in the presence of fibroblast growth factor 2 (FGF-2); (b) incubating the stem cells in the presence of an amount of one or more Neural Development Factors (NDF) sufficient to generate cells expressing Pax-6, Otx2, Nestin, or a combination, thereby producing neural cells, wherein the NDF activate the phosphatidylinositol 3-kinase signaling pathway, inhibit the TGF-β superfamily signaling pathway, and inhibit the Wnt signaling pathway, wherein the NDF comprise Midkine, Pleiotrophin, insulin-like growth factor-1, or a combination; A83-01, SB431542, or a combination; dorsopmorphin; and PNU-74654, Dickkopf, or a combination, wherein prior to incubating in the presence of the NDF, at the same time as incubating in the presence of the NDF is initiated, or during incubating in the presence the NDF the use of FGF-2 is discontinued; and (c) culturing the neural cells, wherein the neural cells are cultured on a treated polymer substrate, wherein the neural cells are cultured in serum free conditions, wherein the neural cells are cultured in the presence of fibroblast growth factor 2 (FGF-2).

The treated polymer substrate can be, for example, CELLBIND™, or substrate treated with Matrigel™, Geltrex™, or fibronectin. The serum free conditions can comprise N2 supplement and B27 supplement. The neural cells can be cultured in the presence of epidermal growth factor (EGF). The neural cells can be cryopreservable. The stem can also be cultured in the presence of conditioned media or chemically defined media such as mouse embryonic fibroblast-conditioned media (MEF-CM), mTeSR™ or StemPro™. The stem cells can also be incubated in the presence of 20% knockout serum replacement (KSR). Use of conditioned media can be discontinued when the use of FGF-2 is discontinued.

The disclosed methods for producing neural cells from stem cells can involve incubation with NDF. The methods can further comprise pre-incubation of the stem cells prior to the incubation. The methods can further comprise culturing the neural cells produced by the incubation. The methods can further comprise a transition from pre-incubation and incubation. The methods can further comprise a transition from incubation and culturing.

The methods can further provide further differentiation of the neural cells during or after culturing. For example, neural stem cells produced during incubation can be differentiated into neural cell lineages, tissue types and/or cell types during or after culturing.

The methods can be performed wherein prior to incubating in the presence of the NDF, the stem cells are cultured in the absence of feeder cells and on an extracellular matrix. The extracellular matrix can be, for example, Matrigel™ or Geltrex™. The methods can be performed wherein prior to incubating in the presence of the NDF the stem cells are cultured on fibroblasts. The fibroblasts can be, for example, from the same species as the stem cells. The fibroblasts can be, for example, from the same subject as the stem cells. The fibroblasts can be, for example, human fibroblasts.

The methods can be performed wherein prior to incubating in the presence of the NDF, the stem cells are cultured in the presence of fibroblast growth factor 2 (FGF-2). The stem can also be cultured in the presence of conditioned media or chemically defined media such as mouse embryonic fibroblast-conditioned media (MEF-CM), mTeSR™ or StemPro™. The methods can be performed wherein prior to incubating in the presence of the NDF, at the same time as incubating in the presence of the NDF is initiated, or during incubating in the presence the NDF the use of FGF-2 is discontinued. The methods can be performed wherein the use of FGF-2 is discontinued at the same time as incubating in the presence of the NDF is initiated. The methods can be performed wherein the use of FGF-2 is discontinued at the same time as incubating in the presence of the NDF is initiated by replacing growth medium containing FGF-2 and lacking NDF with growth medium lacking FGF-2 and containing the NDF. The methods can be performed wherein prior to incubating in the presence of the NDF, at least a portion of the stem cells are cultured to a density of 1×10⁴ cells per square centimeter or greater.

The methods can be performed further comprising culturing the neural cells. The neural cells can be, for example, cultured on a treated polymer substrate. The treated polymer substrate can be, for example, CELLBIND™, or substrate treated with Matrigel™ Geltrex™, or fibronectin. The neural cells can be, for example, cultured in serum free conditions. The serum free conditions can comprise N2 supplement and B27 supplement. The neural cells can be, for example, cultured in the presence of fibroblast growth factor 2 (FGF-2). The neural cells can be cultured in the presence of epidermal growth factor (EGF). The neural cells can be cryopreservable. The neural cells can be, for example, passaged with Accutase or collagenase IV. The stem cells can be, for example, human stem cells. The stem cells can be, for example, embryonic stem cells (ESC). The stem cells can be, for example, derived from embryonic or fetal tissue. The stem cells can be, for example, derived from postfetal tissue. The stem cells can be, for example, derived from adult tissue. The stem cells can be, for example, derived from adult tissue. The stem cells can be, for example, induced pluripotent stem cells (iPSC). The stem cells can be, for example, derived from a subject in need of neural cells.

As used throughout, by a “subject” is meant an individual. Thus, the “subject” can include domesticated animals, such as cats, dogs, etc., livestock (e.g., cattle, horses, pigs, sheep, goats, etc.), laboratory animals (e.g., mouse, rabbit, rat, guinea pig, etc.) and birds. In one aspect, the subject is a mammal such as a primate or a human.

By “treatment” is meant the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventive treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Passage” and “passaging” refer to dissociating cells, and re-plating a number of cells. For example, passage may comprise detaching/dissociating the cells from the culture dish (using, for example trypsin, Accutase, or collagenase IV), dissociating aggregates of cells and re-plating a number of dissociated cells (adherent culture) and culturing the cells.

“Basal medium” refers to a solution of amino acids, vitamins, salts, and nutrients that is effective to support the growth of cells in culture, although normally these compounds will not support cell growth unless supplemented with additional compounds. The nutrients include a carbon source (e.g., a sugar such as glucose) that can be metabolized by the cells, as well as other compounds necessary for the cells' survival. These are compounds that the cells themselves can not synthesize, due to the absence of one or more of the gene(s) that encode the protein(s) necessary to synthesize the compound (e.g., essential amino acids) or, with respect to compounds which the cells can synthesize, because of their particular developmental state the gene(s) encoding the necessary biosynthetic proteins are not being expressed as sufficient levels. A number of base media are known in the art of mammalian cell culture, such as Dulbecco's Modified Eagle Media (DMEM), Knockout-DMEM (KO-DMEM), and DMEM/F12, although any base medium can be used. For growth of stem cells, any medium that can be, for example, supplemented with FGF-2, EGF, insulin, ascorbic acid, and N2 supplement and which supports the growth of stem cells in a substantially undifferentiated state can be employed.

“Conditioned medium” refers to a growth medium that is further supplemented with soluble factors derived from cells cultured in the medium. Techniques for isolating conditioned medium from a cell culture are well known in the art. As will be appreciated, conditioned medium is preferably essentially cell-free. In this context, “essentially cell-free” refers to a conditioned medium that contains fewer than about 10%, preferably fewer than about 5%, 1%, 0.1%, 0.01%, 0.001%, and 0.0001% than the number of cells per unit volume, as compared to the culture from which it was separated.

A “defined” medium refers to a biochemically defined formulation comprised solely of the biochemically-defined constituents. A defined medium can include solely constituents having known chemical compositions. A defined medium can also include constituents that are derived from known sources. For example, a defined medium can also include factors and other compositions secreted from known tissues or cells; however, the defined medium will not include the conditioned medium from a culture of such cells. Thus, a “defined medium” can, if indicated, include a particular compounds added from the culture medium, up to and including a portion of a conditioned medium that has been fractionated to remove at least one component detectable in a sample of the conditioned medium that has not been fractionated. Here, to “substantially remove” of one or more detectable components of a conditioned medium refers to the removal of at least an amount of the detectable, known component(s) from the conditioned medium so as to result in a fractionated conditioned medium that differs from an unfractionated conditioned medium in its ability to support the long-term substantially undifferentiated culture of primate stem cells. Fractionation of a conditioned medium can be performed by any method (or combination of methods) suitable to remove the detectable component(s), for example, gel filtration chromatography, affinity chromatography, immune precipitation, etc. Similarly, a “defined medium” can include serum components derived from an animal, including human serum components. In this context, “known” refers to the knowledge of one of ordinary skill in the art with reference to the chemical composition or constituent.

A cell culture is “essentially feeder-free” when it does not contain exogenously added conditioned medium taken from a culture of feeder cells nor exogenously added feeder cells in the culture, where “no exogenously added feeder cells” means that cells to develop a feeder cell layer have not been purposely introduced for that reason. Of course, if the cells to be cultured are derived from a seed culture that contained feeder cells, the incidental co-isolation and subsequent introduction into another culture of some small proportion of those feeder cells along with the desired cells (e.g., undifferentiated primate primordial stem cells) should not be deemed as an intentional introduction of feeder cells. Similarly, feeder cells or feeder-like cells that develop from stem cells seeded into the culture shall not be deemed to have been purposely introduced into the culture.

A “growth environment” is an environment in which cells (e.g., stem cells and neural stem cells) will proliferate in vitro. Features of the environment include the medium in which the cells are cultured, and a supporting structure (such as a substrate on a solid surface) if present.

Factors can include natural biomolecules that affect cell growth, cell physiology, development, and/or signaling pathways; derivatives, analogs or mimetics of such biomolecules; and compounds that that affect cell growth, cell physiology, development, and/or signaling pathways. Any or all of these types of factors (natural biomolecules, derivatives, analogs or mimetics of such biomolecules, compounds, etc.) can be used alone or in combination. Growth factors include, but are not limited to, for example, fibroblast growth factor 2 (FGF-2), acidic fibroblast growth factor (aFGF), epidermal growth factor (EGF), insulin-like growth factor-I (IGF-I), insulin-like growth factor-II (IGF-II), platelet-derived growth factor-AB (PDGF), vascular endothelial cell growth factor (VEGF), activin-A, bone morphogenic proteins (BMPs), Midkine, Pleiotrophin, insulin-like growth factor-1, Delta-1, Delta-2, Delta-3, Delta-4, Jagged-1, Jagged-2, A83-01, SB431542, dorsopmorphin, PNU-74654, Dickkopf, insulin, cytokines, chemokines, morphogents, neutralizing antibodies, other proteins, and small molecules.

Factors, such as the disclosed Neural Development Factors, can be used at any suitable concentrations. For example, factors can be used at concentrations that produce the intended effect. For example, NDF used to activate the PI3K signaling pathway can be used at concentrations that activate the PI3K signaling pathway, NDF used to activate the MAPK signaling pathway can be used at concentrations that activate the MAPK signaling pathway, NDF used to activate the PI3K and MAPK signaling pathways can be used at concentrations that activate the PI3K and MAPK signaling pathways, NDF used to inhibit the TGF-β superfamily signaling pathway can be used at concentrations that inhibit the TGF-β superfamily signaling pathway, NDF used to inhibit the Wnt signaling pathway can be used at concentrations that inhibit the Wnt signaling pathway, etc. Such concentrations are exemplified in the Examples herein, are generally known to those of skill in the art, and/or can be determined by measuring the desired effect (such as activation or inhibition of the relevant signaling pathway) using techniques describe herein or known to those of skill in the art.

In general, concentrations of protein and peptide factors can be expressed in ng/ml and concentrations of small molecule factors can be expressed in μM, although each can be exchanged and, as may be appropriate, different units, such as mg/ml or nM, for example, can be used. Some factors, such as Noggin, may need to be used at supraphysiological concentrations (200 ng/ml and above, such as 500 ng/ml for Noggin).

FGF-2, Midkine, Pleiotrophin, IGF-1, EGF, and Dickkopf, for example, can be used at concentrations of, for example, 10 ng/ml, 15 ng/ml, 20 ng/ml, 30 ng/ml, 40 ng/ml, 50 ng/ml, 60 ng/ml, 70 ng/ml, 80 ng/ml, 90 ng/ml, 100 ng/ml, 120 ng/ml, 140 ng/ml, 160 ng/ml, 180 ng/ml, or 200 ng/ml. Dorsomorphin, A83-01, SB431542, and PNU-74654, for example, can be used at concentrations of, for example, 10 nM, 20 nM, 30 nM, 40 nM, 50 nM, 60 nM, 70 nM, 80 nM, 90 nM, 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, 10 μM, 15 μM, 20 μM, 30 μM, 40 μM, 50 μM, 60 μM, 70 μM, 80 μM, 90 μM, or 100 μM.

In general, factors such as the disclosed NDF can be used at concentrations of 2 ng/ml, 3 ng/ml, 4 ng/ml, 5 ng/ml, 6 ng/ml, 7 ng/ml, 8 ng/ml, 9 ng/ml, 10 ng/ml, 15 ng/ml, 20 ng/ml, 30 ng/ml, 40 ng/ml, 50 ng/ml, 60 ng/ml, 70 ng/ml, 80 ng/ml, 90 ng/ml, 100 ng/ml, 120 ng/ml, 140 ng/ml, 160 ng/ml, 180 ng/ml, 200 ng/ml, 2 to 200 ng/ml, 3 to 200 ng/ml, 4 to 200 ng/ml, 5 to 200 ng/ml, 6 to 200 ng/ml, 7 to 200 ng/ml, 8 to 200 ng/ml, 9 to 200 ng/ml, 10 to 200 ng/ml, 15 to 200 ng/ml, 20 to 200 ng/ml, 30 to 200 ng/ml, 40 to 200 ng/ml, 50 to 200 ng/ml, 60 to 200 ng/ml, 70 to 200 ng/ml, 80 to 200 ng/ml, 90 to 200 ng/ml, 100 to 200 ng/ml, 120 to 200 ng/ml, 140 to 200 ng/ml, 160 to 200 ng/ml, 180 to 200 ng/ml, 2 to 180 ng/ml, 3 to 180 ng/ml, 4 to 180 ng/ml, 5 to 180 ng/ml, 6 to 180 ng/ml, 7 to 180 ng/ml, 8 to 180 ng/ml, 9 to 180 ng/ml, 10 to 180 ng/ml, 15 to 180 ng/ml, 20 to 180 ng/ml, 30 to 180 ng/ml, 40 to 180 ng/ml, 50 to 180 ng/ml, 60 to 180 ng/ml, 70 to 180 ng/ml, 80 to 180 ng/ml, 90 to 180 ng/ml, 100 to 180 ng/ml, 120 to 180 ng/ml, 140 to 180 ng/ml, 160 to 180 ng/ml, 2 to 160 ng/ml, 3 to 160 ng/ml, 4 to 160 ng/ml, 5 to 160 ng/ml, 6 to 160 ng/ml, 7 to 160 ng/ml, 8 to 160 ng/ml, 9 to 160 ng/ml, 10 to 160 ng/ml, 15 to 160 ng/ml, 20 to 160 ng/ml, 30 to 160 ng/ml, 40 to 160 ng/ml, 50 to 160 ng/ml, 60 to 160 ng/ml, 70 to 160 ng/ml, 80 to 160 ng/ml, 90 to 160 ng/ml, 100 to 160 ng/ml, 120 to 160 ng/ml, 140 to 160 ng/ml, 2 to 140 ng/ml, 3 to 140 ng/ml, 4 to 140 ng/ml, 5 to 140 ng/ml, 6 to 140 ng/ml, 7 to 140 ng/ml, 8 to 140 ng/ml, 9 to 140 ng/ml, 10 to 140 ng/ml, 15 to 140 ng/ml, 20 to 140 ng/ml, 30 to 140 ng/ml, 40 to 140 ng/ml, 50 to 140 ng/ml, 60 to 140 ng/ml, 70 to 140 ng/ml, 80 to 140 ng/ml, 90 to 140 ng/ml, 100 to 140 ng/ml, 120 to 140 ng/ml, 2 to 120 ng/ml, 3 to 120 ng/ml, 4 to 120 ng/ml, 5 to 120 ng/ml, 6 to 120 ng/ml, 7 to 120 ng/ml, 8 to 120 ng/ml, 9 to 120 ng/ml, 10 to 120 ng/ml, 15 to 120 ng/ml, 20 to 120 ng/ml, 30 to 120 ng/ml, 40 to 120 ng/ml, 50 to 120 ng/ml, 60 to 120 ng/ml, 70 to 120 ng/ml, 80 to 120 ng/ml, 90 to 120 ng/ml, 100 to 120 ng/ml, 2 to 100 ng/ml, 3 to 100 ng/ml, 4 to 100 ng/ml, 5 to 100 ng/ml, 6 to 100 ng/ml, 7 to 100 ng/ml, 8 to 100 ng/ml, 9 to 100 ng/ml, 10 to 100 ng/ml, 15 to 100 ng/ml, 20 to 100 ng/ml, 30 to 100 ng/ml, 40 to 100 ng/ml, 50 to 100 ng/ml, 60 to 100 ng/ml, 70 to 100 ng/ml, 80 to 100 ng/ml, 90 to 100 ng/ml, 2 to 90 ng/ml, 3 to 90 ng/ml, 4 to 90 ng/ml, 5 to 90 ng/ml, 6 to 90 ng/ml, 7 to 90 ng/ml, 8 to 90 ng/ml, 9 to 90 ng/ml, 10 to 90 ng/ml, 15 to 90 ng/ml, 20 to 90 ng/ml, 30 to 90 ng/ml, 40 to 90 ng/ml, 50 to 90 ng/ml, 60 to 90 ng/ml, 70 to 90 ng/ml, 80 to 90 ng/ml, 2 to 80 ng/ml, 3 to 80 ng/ml, 4 to 80 ng/ml, 5 to 80 ng/ml, 6 to 80 ng/ml, 7 to 80 ng/ml, 8 to 80 ng/ml, 9 to 80 ng/ml, 10 to 80 ng/ml, 15 to 80 ng/ml, 20 to 80 ng/ml, 30 to 80 ng/ml, 40 to 80 ng/ml, 50 to 80 ng/ml, 60 to 80 ng/ml, 70 to 80 ng/ml, 2 to 70 ng/ml, 3 to 70 ng/ml, 4 to 70 ng/ml, 5 to 70 ng/ml, 6 to 70 ng/ml, 7 to 70 ng/ml, 8 to 70 ng/ml, 9 to 70 ng/ml, 10 to 70 ng/ml, 15 to 70 ng/ml, 20 to 70 ng/ml, 30 to 70 ng/ml, 40 to 70 ng/ml, 50 to 70 ng/ml, 60 to 70 ng/ml, 2 to 60 ng/ml, 3 to 60 ng/ml, 4 to 60 ng/ml, 5 to 60 ng/ml, 6 to 60 ng/ml, 7 to 60 ng/ml, 8 to 60 ng/ml, 9 to 60 ng/ml, 10 to 60 ng/ml, 15 to 60 ng/ml, 20 to 60 ng/ml, 30 to 60 ng/ml, 40 to 60 ng/ml, 50 to 60 ng/ml, 2 to 50 ng/ml, 3 to 50 ng/ml, 4 to 50 ng/ml, 5 to 50 ng/ml, 6 to 50 ng/ml, 7 to 50 ng/ml, 8 to 50 ng/ml, 9 to 50 ng/ml, 10 to 50 ng/ml, 15 to 50 ng/ml, 20 to 50 ng/ml, 30 to 50 ng/ml, 40 to 50 ng/ml, 2 to 40 ng/ml, 3 to 40 ng/ml, 4 to 40 ng/ml, 5 to 40 ng/ml, 6 to 40 ng/ml, 7 to 40 ng/ml, 8 to 40 ng/ml, 9 to 40 ng/ml, 10 to 40 ng/ml, 15 to 40 ng/ml, 20 to 40 ng/ml, 30 to 40 ng/ml, 2 to 30 ng/ml, 3 to 30 ng/ml, 4 to 30 ng/ml, 5 to 30 ng/ml, 6 to 30 ng/ml, 7 to 30 ng/ml, 8 to 30 ng/ml, 9 to 30 ng/ml, 10 to 30 ng/ml, 15 to 30 ng/ml, 20 to 30 ng/ml, 2 to 20 ng/ml, 3 to 20 ng/ml, 4 to 20 ng/ml, 5 to 20 ng/ml, 6 to 20 ng/ml, 7 to 20 ng/ml, 8 to 20 ng/ml, 9 to 20 ng/ml, 10 to 20 ng/ml, 15 to 20 ng/ml, 2 to 15 ng/ml, 3 to 15 ng/ml, 4 to 15 ng/ml, 5 to 15 ng/ml, 6 to 15 ng/ml, 7 to 15 ng/ml, 8 to 15 ng/ml, 9 to 15 ng/ml, 10 to 15 ng/ml, 2 to 10 ng/ml, 3 to 10 ng/ml, 4 to 10 ng/ml, 5 to 10 ng/ml, 6 to 10 ng/ml, 7 to 10 ng/ml, 8 to 10 ng/ml, 9 to 10 ng/ml, 2 to 9 ng/ml, 3 to 9 ng/ml, 4 to 9 ng/ml, 5 to 9 ng/ml, 6 to 9 ng/ml, 7 to 9 ng/ml, 8 to 9 ng/ml, 2 to 8 ng/ml, 3 to 8 ng/ml, 4 to 8 ng/ml, 5 to 8 ng/ml, 6 to 8 ng/ml, 7 to 8 ng/ml, 2 to 7 ng/ml, 3 to 7 ng/ml, 4 to 7 ng/ml, 5 to 7 ng/ml, 6 to 7 ng/ml, 2 to 6 ng/ml, 3 to 6 ng/ml, 4 to 6 ng/ml, 5 to 6 ng/ml, 2 to 5 ng/ml, 3 to 5 ng/ml, 4 to 5 ng/ml, 2 to 4 ng/ml, 3 to 4 ng/ml, or 2 to 3 ng/ml.

Factors such as the disclosed NDF can be used at concentrations of 10 nM, 20 nM, 30 nM, 40 nM, 50 nM, 60 nM, 70 nM, 80 nM, 90 nM, 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, 10 μM, 15 μM, 20 μM, 30 μM, 40 μM, 50 μM, 60 μM, 70 μM, 80 μM, 90 μM, 100 μM, 120 μM, 140 μM, 160 μM, 180 μM, 200 μM, 10 to 200 nM, 20 to 200 nM, 30 to 200 nM, 40 to 200 nM, 50 to 200 nM, 60 to 200 nM, 70 to 200 nM, 80 to 200 nM, 90 to 200 nM, 0.1 to 200 μM, 0.2 to 200 μM, 0.3 to 200 μM, 0.4 to 200 μM, 0.5 to 200 μM, 0.6 to 200 μM, 0.7 to 200 μM, 0.8 to 200 μM, 0.9 to 200 μM, 1 to 200 μM, 2 to 200 μM, 3 to 200 μM, 4 to 200 μM, 5 to 200 μM, 6 to 200 μM, 7 to 200 μM, 8 to 200 μM, 9 to 200 μM, 10 to 200 μM, 15 to 200 μM, 20 to 200 μM, 30 to 200 μM, 40 to 200 μM, 50 to 200 μM, 60 to 200 μM, 70 to 200 μM, 80 to 200 μM, 90 to 200 μM, 100 to 200 μM, 120 to 200 μM, 140 to 200 μM, 160 to 200 μM, 180 to 200 μM, 10 to 180 nM, 20 to 180 nM, 30 to 180 nM, 40 to 180 nM, 50 to 180 nM, 60 to 180 nM, 70 to 180 nM, 80 to 180 nM, 90 to 180 nM, 0.1 to 180 μM, 0.2 to 180 μM, 0.3 to 180 μM, 0.4 to 180 μM, 0.5 to 180 μM, 0.6 to 180 μM, 0.7 to 180 μM, 0.8 to 180 μM, 0.9 to 180 μM, 1 to 180 μM, 2 to 180 μM, 3 to 180 μM, 4 to 180 μM, 5 to 180 μM, 6 to 180 μM, 7 to 180 μM, 8 to 180 μM, 9 to 180 μM, 10 to 180 μM, 15 to 180 μM, 20 to 180 μM, 30 to 180 μM, 40 to 180 μM, 50 to 180 μM, 60 to 180 μM, 70 to 180 μM, 80 to 180 μM, 90 to 180 μM, 100 to 180 μM, 120 to 180 μM, 140 to 180 μM, 160 to 180 μM, 10 to 160 nM, 20 to 160 nM, 30 to 160 nM, 40 to 160 nM, 50 to 160 nM, 60 to 160 nM, 70 to 160 nM, 80 to 160 nM, 90 to 160 nM, 0.1 to 160 μM, 0.2 to 160 μM, 0.3 to 160 μM, 0.4 to 160 μM, 0.5 to 160 μM, 0.6 to 160 μM, 0.7 to 160 μM, 0.8 to 160 μM, 0.9 to 160 μM, 1 to 160 μM, 2 to 160 μM, 3 to 160 μM, 4 to 160 μM, 5 to 160 μM, 6 to 160 μM, 7 to 160 μM, 8 to 160 μM, 9 to 160 μM, 10 to 160 μM, 15 to 160 μM, 20 to 160 μM, 30 to 160 μM, 40 to 160 μM, 50 to 160 μM, 60 to 160 μM, 70 to 160 μM, 80 to 160 μM, 90 to 160 μM, 100 to 160 μM, 120 to 160 μM, 140 to 160 μM, 10 to 140 nM, 20 to 140 nM, 30 to 140 nM, 40 to 140 nM, 50 to 140 nM, 60 to 140 nM, 70 to 140 nM, 80 to 140 nM, 90 to 140 nM, 0.1 to 140 μM, 0.2 to 140 μM, 0.3 to 140 μM, 0.4 to 140 μM, 0.5 to 140 μM, 0.6 to 140 μM, 0.7 to 140 μM, 0.8 to 140 μM, 0.9 to 140 μM, 1 to 140 μM, 2 to 140 μM, 3 to 140 μM, 4 to 140 μM, 5 to 140 μM, 6 to 140 μM, 7 to 140 μM, 8 to 140 μM, 9 to 140 μM, 10 to 140 μM, 15 to 140 μM, 20 to 140 μM, 30 to 140 μM, 40 to 140 μM, 50 to 140 μM, 60 to 140 μM, 70 to 140 μM, 80 to 140 μM, 90 to 140 μM, 100 to 140 μM, 120 to 140 μM, 10 to 120 nM, 20 to 120 nM, 30 to 120 nM, 40 to 120 nM, 50 to 120 nM, 60 to 120 nM, 70 to 120 nM, 80 to 120 nM, 90 to 120 nM, 0.1 to 120 μM, 0.2 to 120 μM, 0.3 to 120 μM, 0.4 to 120 μM, 0.5 to 120 μM, 0.6 to 120 μM, 0.7 to 120 μM, 0.8 to 120 μM, 0.9 to 120 μM, 1 to 120 μM, 2 to 120 μM, 3 to 120 μM, 4 to 120 μM, 5 to 120 μM, 6 to 120 μM, 7 to 120 μM, 8 to 120 μM, 9 to 120 μM, 10 to 120 μM, 15 to 120 μM, 20 to 120 μM, 30 to 120 μM, 40 to 120 μM, 50 to 120 μM, 60 to 120 μM, 70 to 120 μM, 80 to 120 μM, 90 to 120 μM, 100 to 120 μM, 10 to 100 nM, 20 to 100 nM, 30 to 100 nM, 40 to 100 nM, 50 to 100 nM, 60 to 100 nM, 70 to 100 nM, 80 to 100 nM, 90 to 100 nM, 0.1 to 100 μM, 0.2 to 100 μM, 0.3 to 100 μM, 0.4 to 100 μM, 0.5 to 100 μM, 0.6 to 100 μM, 0.7 to 100 μM, 0.8 to 100 μM, 0.9 to 100 μM, 1 to 100 μM, 2 to 100 μM, 3 to 100 μM, 4 to 100 μM, 5 to 100 μM, 6 to 100 μM, 7 to 100 μM, 8 to 100 μM, 9 to 100 μM, 10 to 100 μM, 15 to 100 μM, 20 to 100 μM, 30 to 100 μM, 40 to 100 μM, 50 to 100 μM, 60 to 100 μM, 70 to 100 μM, 80 to 100 μM, 90 to 100 μM, 10 to 90 nM, 20 to 90 nM, 30 to 90 nM, 40 to 90 nM, 50 to 90 nM, 60 to 90 nM, 70 to 90 nM, 80 to 90 nM, 9 to 90 nM, 0.1 to 90 μM, 0.2 to 90 μM, 0.3 to 90 μM, 0.4 to 90 μM, 0.5 to 90 μM, 0.6 to 90 μM, 0.7 to 90 μM, 0.8 to 90 μM, 0.9 to 90 μM, 1 to 90 μM, 2 to 90 μM, 3 to 90 μM, 4 to 90 μM, 5 to 90 μM, 6 to 90 μM, 7 to 90 μM, 8 to 90 μM, 9 to 90 μM, 10 to 90 μM, 15 to 90 μM, 20 to 90 μM, 30 to 90 μM, 40 to 90 μM, 50 to 90 μM, 60 to 90 μM, 70 to 90 μM, 80 to 90 μM, 10 to 80 nM, 20 to 80 nM, 30 to 80 nM, 40 to 80 nM, 50 to 80 nM, 60 to 80 nM, 70 to 80 nM, 8 to 80 nM, 9 to 80 nM, 0.1 to 80 μM, 0.2 to 80 μM, 0.3 to 80 μM, 0.4 to 80 μM, 0.5 to 80 μM, 0.6 to 80 μM, 0.7 to 80 μM, 0.8 to 80 μM, 0.9 to 80 μM, 1 to 80 μM, 2 to 80 μM, 3 to 80 μM, 4 to 80 μM, 5 to 80 μM, 6 to 80 μM, 7 to 80 μM, 8 to 80 μM, 9 to 80 μM, 10 to 80 μM, 15 to 80 μM, 20 to 80 μM, 30 to 80 μM, 40 to 80 μM, 50 to 80 μM, 60 to 80 μM, 70 to 80 μM, 10 to 70 nM, 20 to 70 nM, 30 to 70 nM, 40 to 70 nM, 50 to 70 nM, 60 to 70 nM, 7 to 70 nM, 8 to 70 nM, 9 to 70 nM, 0.1 to 70 μM, 0.2 to 70 μM, 0.3 to 70 μM, 0.4 to 70 μM, 0.5 to 70 μM, 0.6 to 70 μM, 0.7 to 70 μM, 0.8 to 70 μM, 0.9 to 70 μM, 1 to 70 μM, 2 to 70 μM, 3 to 70 μM, 4 to 70 μM, 5 to 70 μM, 6 to 70 μM, 7 to 70 μM, 8 to 70 μM, 9 to 70 μM, 10 to 70 μM, 15 to 70 μM, 20 to 70 μM, 30 to 70 μM, 40 to 70 μM, 50 to 70 μM, 60 to 70 μM, 10 to 60 nM, 20 to 60 nM, 30 to 60 nM, 40 to 60 nM, 50 to 60 nM, 6 to 60 nM, 7 to 60 nM, 8 to 60 nM, 9 to 60 nM, 0.1 to 60 μM, 0.2 to 60 μM, 0.3 to 60 μM, 0.4 to 60 μM, 0.5 to 60 μM, 0.6 to 60 μM, 0.7 to 60 μM, 0.8 to 60 μM, 0.9 to 60 μM, 1 to 60 μM, 2 to 60 μM, 3 to 60 μM, 4 to 60 μM, 5 to 60 μM, 6 to 60 μM, 7 to 60 μM, 8 to 60 μM, 9 to 60 μM, 10 to 60 μM, 15 to 60 μM, 20 to 60 μM, 30 to 60 μM, 40 to 60 μM, 50 to 60 μM, 10 to 50 nM, 20 to 50 nM, 30 to 50 nM, 40 to 50 nM, 5 to 50 nM, 6 to 50 nM, 7 to 50 nM, 8 to 50 nM, 9 to 50 nM, 0.1 to 50 μM, 0.2 to 50 μM, 0.3 to 50 μM, 0.4 to 50 μM, 0.5 to 50 μM, 0.6 to 50 μM, 0.7 to 50 μM, 0.8 to 50 μM, 0.9 to 50 μM, 1 to 50 μM, 2 to 50 μM, 3 to 50 μM, 4 to 50 μM, 5 to 50 μM, 6 to 50 μM, 7 to 50 μM, 8 to 50 μM, 9 to 50 μM, 10 to 50 μM, 15 to 50 μM, 20 to 50 μM, 30 to 50 μM, 40 to 50 μM, 10 to 40 nM, 20 to 40 nM, 30 to 40 nM, 4 to 40 nM, 5 to 40 nM, 6 to 40 nM, 7 to 40 nM, 8 to 40 nM, 9 to 40 nM, 0.1 to 40 μM, 0.2 to 40 μM, 0.3 to 40 μM, 0.4 to 40 μM, 0.5 to 40 μM, 0.6 to 40 μM, 0.7 to 40 μM, 0.8 to 40 μM, 0.9 to 40 μM, 1 to 40 μM, 2 to 40 μM, 3 to 40 μM, 4 to 40 μM, 5 to 40 μM, 6 to 40 μM, 7 to 40 μM, 8 to 40 μM, 9 to 40 μM, 10 to 40 μM, 15 to 40 μM, 20 to 40 μM, 30 to 40 μM, 10 to 30 nM, 20 to 30 nM, 3 to 30 nM, 4 to 30 nM, 5 to 30 nM, 6 to 30 nM, 7 to 30 nM, 8 to 30 nM, 9 to 30 nM, 0.1 to 30 μM, 0.2 to 30 μM, 0.3 to 30 μM, 0.4 to 30 μM, 0.5 to 30 μM, 0.6 to 30 μM, 0.7 to 30 μM, 0.8 to 30 μM, 0.9 to 30 μM, 1 to 30 μM, 2 to 30 μM, 3 to 30 μM, 4 to 30 μM, 5 to 30 μM, 6 to 30 μM, 7 to 30 μM, 8 to 30 μM, 9 to 30 μM, 10 to 30 μM, 15 to 30 μM, 20 to 30 μM, 10 to 20 nM, 2 to 20 nM, 3 to 20 nM, 4 to 20 nM, 5 to 20 nM, 6 to 20 nM, 7 to 20 nM, 8 to 20 nM, 9 to 20 nM, 0.1 to 20 μM, 0.2 to 20 μM, 0.3 to 20 μM, 0.4 to 20 μM, 0.5 to 20 μM, 0.6 to 20 μM, 0.7 to 20 μM, 0.8 to 20 μM, 0.9 to 20 μM, 1 to 20 μM, 2 to 20 μM, 3 to 20 μM, 4 to 20 μM, 5 to 20 μM, 6 to 20 μM, 7 to 20 μM, 8 to 20 μM, 9 to 20 μM, 10 to 20 μM, 15 to 20 μM, 10 to 15 nM, 2 to 15 nM, 3 to 15 nM, 4 to 15 nM, 5 to 15 nM, 6 to 15 nM, 7 to 15 nM, 8 to 15 nM, 9 to 15 nM, 0.1 to 15 μM, 0.2 to 15 μM, 0.3 to 15 μM, 0.4 to 15 μM, 0.5 to 15 μM, 0.6 to 15 μM, 0.7 to 15 μM, 0.8 to 15 μM, 0.9 to 15 μM, 1 to 15 μM, 2 to 15 μM, 3 to 15 μM, 4 to 15 μM, 5 to 15 μM, 6 to 15 μM, 7 to 15 μM, 8 to 15 μM, 9 to 15 μM, 10 to 15 μM, 1 to 10 nM, 2 to 10 nM, 3 to 10 nM, 4 to 10 nM, 5 to 10 nM, 6 to 10 nM, 7 to 10 nM, 8 to 10 nM, 9 to 10 nM, 0.1 to 10 μM, 0.2 to 10 μM, 0.3 to 10 μM, 0.4 to 10 μM, 0.5 to 10 μM, 0.6 to 10 μM, 0.7 to 10 μM, 0.8 to 10 μM, 0.9 to 10 μM, 1 to 10 μM, 2 to 10 μM, 3 to 10 μM, 4 to 10 μM, 5 to 10 μM, 6 to 10 μM, 7 to 10 μM, 8 to 10 μM, 9 to 10 μM, 0.1 to 9 μM, 0.2 to 9 μM, 0.3 to 9 μM, 0.4 to 9 μM, 0.5 to 9 μM, 0.6 to 9 μM, 0.7 to 9 μM, 0.8 to 9 μM, 0.9 to 9 μM, 1 to 9 μM, 2 to 9 μM, 3 to 9 μM, 4 to 9 μM, 5 to 9 μM, 6 to 9 μM, 7 to 9 μM, 8 to 9 μM, 0.1 to 8 μM, 0.2 to 8 μM, 0.3 to 8 μM, 0.4 to 8 μM, 0.5 to 8 μM, 0.6 to 8 μM, 0.7 to 8 μM, 0.8 to 8 μM, 0.9 to 8 μM, 1 to 8 μM, 2 to 8 μM, 3 to 8 μM, 4 to 8 μM, 5 to 8 μM, 6 to 8 μM, 7 to 8 μM, 0.1 to 7 μM, 0.2 to 7 μM, 0.3 to 7 μM, 0.4 to 7 μM, 0.5 to 7 μM, 0.6 to 7 μM, 0.7 to 7 μM, 0.8 to 7 μM, 0.9 to 7 μM, 1 to 7 μM, 2 to 7 μM, 3 to 7 μM, 4 to 7 μM, 5 to 7 μM, 6 to 7 μM, 0.1 to 6 μM, 0.2 to 6 μM, 0.3 to 6 μM, 0.4 to 6 μM, 0.5 to 6 μM, 0.6 to 6 μM, 0.7 to 6 μM, 0.8 to 6 μM, 0.9 to 6 μM, 1 to 6 μM, 2 to 6 μM, 3 to 6 μM, 4 to 6 μM, 5 to 6 μM, 0.1 to 5 μM, 0.2 to 5 μM, 0.3 to 5 μM, 0.4 to 5 μM, 0.5 to 5 μM, 0.6 to 5 μM, 0.7 to 5 μM, 0.8 to 5 μM, 0.9 to 5 μM, 1 to 5 μM, 2 to 5 μM, 3 to 5 μM, 4 to 5 μM, 0.1 to 4 μM, 0.2 to 4 μM, 0.3 to 4 μM, 0.4 to 4 μM, 0.5 to 4 μM, 0.6 to 4 μM, 0.7 to 4 μM, 0.8 to 4 μM, 0.9 to 4 μM, 1 to 4 μM, 2 to 4 μM, 3 to 4 μM, 0.1 to 3 μM, 0.2 to 3 μM, 0.3 to 3 μM, 0.4 to 3 μM, 0.5 to 3 μM, 0.6 to 3 μM, 0.7 to 3 μM, 0.8 to 3 μM, 0.9 to 3 μM, 1 to 3 μM, 2 to 3 μM, 0.1 to 2 μM, 0.2 to 2 μM, 0.3 to 2 μM, 0.4 to 2 μM, 0.5 to 2 μM, 0.6 to 2 μM, 0.7 to 2 μM, 0.8 to 2 μM, 0.9 to 2 μM, 1 to 2 μM, 0.1 to 1 μM, 0.2 to 1 μM, 0.3 to 1 μM, 0.4 to 1 μM, 0.5 to 1 μM, 0.6 to 1 μM, 0.7 to 1 μM, 0.8 to 1 μM, 0.9 to 1 μM, 0.1 to 0.9 μM, 0.2 to 0.9 μM, 0.3 to 0.9 μM, 0.4 to 0.9 μM, 0.5 to 0.9 μM, 0.6 to 0.9 μM, 0.7 to 0.9 μM, 0.8 to 0.9 μM, 0.1 to 0.8 μM, 0.2 to 0.8 μM, 0.3 to 0.8 μM, 0.4 to 0.8 μM, 0.5 to 0.8 μM, 0.6 to 0.8 μM, 0.7 to 0.8 μM, 0.1 to 0.7 μM, 0.2 to 0.7 μM, 0.3 to 0.7 μM, 0.4 to 0.7 μM, 0.5 to 0.7 μM, 0.6 to 0.7 μM, 0.1 to 0.6 μM, 0.2 to 0.6 μM, 0.3 to 0.6 μM, 0.4 to 0.6 μM, 0.5 to 0.6 μM, 0.1 to 0.5 μM, 0.2 to 0.5 μM, 0.3 to 0.5 μM, 0.4 to 0.5 μM, 0.1 to 0.4 μM, 0.2 to 0.4 μM, 0.3 to 0.4 μM, 0.1 to 0.3 μM, 0.2 to 0.3 μM, or 0.1 to 0.2 μM.

“Isotonic” refers to a solution having essentially the same tonicity (i.e., effective osmotic pressure equivalent) as another solution with which it is compared. In the context of cell culture, an “isotonic” medium is one in which cells can be cultured without an appreciable net flow of water across the cell membranes.

A solution having “low osmotic pressure” refers to a solution having an osmotic pressure of less than about 300 milli-osmols per kilogram (“mOsm/kg”).

A “non-essential amino acid” refers to an amino acid species that need not be added to a culture medium for a given cell type, typically because the cell synthesizes, or is capable of synthesizing, the particular amino acid species. While differing from species to species, non-essential amino acids for primates are known to include L-alanine, L-asparagine, L-aspartic acid, L-glutamic acid, glycine, L-proline, and L-serine.

A cell culture is “essentially serum-free” when it does not contain exogenously added serum, where no “exogenously added feeder cells” means that serum has not been purposely introduced into the medium. Of course, if the cells being cultured produce some or all of the components of serum, of if the cells to be cultured are derived from a seed culture grown in a medium that contained serum, the incidental co-isolation and subsequent introduction into another culture of some small amount of serum (e.g., less than about 1%) should not be deemed as an intentional introduction of serum. The serum free conditions can comprise N2 supplement and B27 supplement.

In some forms of the methods, the cells can be cultured in a culture vessel that contains a substrate such as feeder cells, such as allogeneic feeder cells; an extracellular matrix; a suitable surface; a mixture of factors that adequately activate or inhibit the signal transduction pathways required for, for example, undifferentiated growth (for stem cells), neural induction (to produce neural stem cells), maintenance of neural stem cells, or differentiation (of neural cells, for example); a solution-borne matrix sufficient to support growth of the cells in solution; or a combination. Thus, in addition to the components of the solution phase of culture media, the growth environment can include a substrate such as feeder cells, such as allogeneic feeder cells, and an extracellular matrix, such as laminin, Matrigel™, or Geltrex™.

Useful feeder cells for mammalian cells and mammalian stem cells include, for example, mammalian fibroblasts and stromal cells. Useful feeder cells for primate cells and primate stem cells include, for example, primate fibroblasts and stromal cells. In some forms of the methods, the feeder cells and stem cells, neural stem cells, and neural cells can be allogeneic. In the context of human cells and human stem cells, particularly useful feeder cells include human fibroblasts, human stromal cells, and fibroblast-like cells derived from human stem cells, such as human embryonic stem cells. If living feeder cells are used, as opposed to a synthetic or purified extracellular matrix or a matrix prepared from lysed cells, the cells can be mitotically inactivated (e.g., by irradiation or chemically) to prevent their further growth during the culturing of the cells. Inactivation is usefully performed before seeding the cells into the culture vessel to be used. The cells or stem cells can then be grown on the plate in addition to the feeder cells. Alternatively, the feeder cells can be first grown to confluence and then inactivated to prevent their further growth. If desired, the feeder cells can be stored frozen in liquid nitrogen or at −140° C. prior to use. If desired such a feeder cell layer can be lysed using any suitable technique prior to the addition of the cells or stem cells so as to leave only an extracellular matrix.

Not wishing to be bound to any theory, it is believed that the use of such feeder cells, or an extracellular matrix derived from feeder cells, provides one or more substances necessary to promote the growth of stem cells (e.g., primate primordial stem cells) and/or prevent or decrease the rate of differentiation of such cells. Such substances are believed to include membrane-bound and/or soluble cell products that are secreted into the surrounding medium by the feeder cells. Thus, those skilled in the art will recognize that additional cell lines can be used with the cell culture media to equivalent effect, and that such additional cell lines can be identified using standard methods and materials, for example, by culturing over time (e.g., several passages), for example, substantially undifferentiated stem cells on such feeder cells in a culture medium and determining whether the stem cells remain substantially undifferentiated over the course of the analysis.

When purified components from extracellular matrices are used, such components can include, for example, those provided by the extracellular matrix of a suitable feeder cell layer.

Components of extracellular matrices that can be used include laminin, or products that contain laminin, such as Matrigel™, or other molecules that activate the laminin receptor and/or its downstream signaling pathway. Other extracellular matrix components include fibronectin, collagen, and gelatin. In addition, one or more substances produced by the feeder cells, or contained in an extracellular matrix produced by a feeder cell line, can be identified and used to make a substrate that obviates the need for feeder cells. Alternatively, these components can be prepared in soluble form so as to allow the growth and maintenance of, for example, undifferentiated of stem cells in suspension culture. Thus, extracellular matrix can be added to the fluid phase of a culture at the time of passaging the cells or as part of a regular feeding, as well as preparing the substrate prior to addition of the fluid components of the culture.

Any suitable culture vessel can be adapted to culture the disclosed cells and stem cells. For example, vessels having a substrate suitable for matrix attachment include tissue culture plates (including multi-well plates), pre-coated (e.g., gelatin—pre-coated) plates, T-flasks, roller bottles, gas permeable containers, and bioreactors. To increase efficiency and cell density, vessels (e.g., stirred tanks) that employ suspended particles (e.g., plastic beads or other microcarriers) that can serve as a substrate for attachment of feeder cells or an extracellular matrix can be employed. The cells can be cultured on treated polymer substrate, such as CELLBIND™ (Corning), or substrate treated with Matrigel™, Geltrex™, or fibronectin. In other forms of the disclosed methods, the disclosed cells can be cultured in suspension by providing the matrix components in soluble form. As will be appreciated, fresh medium can be introduced into any of these vessels by batch exchange (replacement of spent medium with fresh medium), fed-batch processes (i.e., fresh medium is added without removal of spent medium), or ongoing exchange in which a proportion of the medium is replaced with fresh medium on a continuous or periodic basis.

### A. Producing Neural Cells

Disclosed are methods for producing neural cells from stem cells. For example, the disclosed methods involve generation of neural stem cells from pluripotent stem cells. For example, disclosed are methods of producing neural cells, the method comprising incubating pluripotent stem cells in the presence of an amount of one or more Neural Development Factors (NDF) sufficient to generate cells expressing Pax-6, Otx2, Nestin, or a combination, thereby producing neural cells. Also disclosed are neural cells produced by one or more of the disclosed methods. Also disclosed are neural cells produced by the method comprising incubating pluripotent stem cells in the presence of an amount of one or more Neural Development Factors (NDF) sufficient to generate cells expressing Pax-6, Otx2, Nestin, or a combination.

Also disclosed are methods of producing neural cells, the method comprising incubating pluripotent stem cells in the presence of an amount of one or more Neural Development Factors (NDF) sufficient to generate cells expressing Pax-6, Otx2, Nestin, or a combination, thereby producing neural cells, wherein the NDF activate the phosphatidylinositol 3-kinase signaling pathway, inhibit the TGF-β superfamily signaling pathway, and inhibit the Wnt signaling pathway, wherein the NDF comprise Midkine, Pleiotrophin, insulin-like growth factor-1, or a combination; A83-01, SB431542, or a combination; dorsopmorphin; and PNU-74654, Dickkopf, or a combination. The stem cells can also be incubated in the presence of 20% knockout serum replacement (KSR).

For example, pluripotent stem cells can be incubated in the presence of 40 to 150 ng/ml of Midkine, 0.05 to 2 μM A83-01, 0.05 to 2 μM dorsopmorphin, and 0.05 to 2 μM PNU-74654. As another example, example, pluripotent stem cells can be incubated in the presence of 80 to 120 ng/ml of Midkine, 0.1 to 0.4 μM A83-01, 0.1 to 0.4 μM dorsopmorphin, and 0.1 to 0.4 μM PNU-74654. As another example, example, pluripotent stem cells can be incubated in the presence of 100 ng/ml of Midkine, 0.2 μM A83-01, 0.2 μM dorsopmorphin, and 0.2 μM PNU-74654. As another example, pluripotent stem cells can be incubated in the presence of 40 to 150 ng/ml of IGF-1, 0.05 to 2 μM A83-01, 0.05 to 2 μM dorsopmorphin, and 0.05 to 2 μM PNU-74654. As another example, example, pluripotent stem cells can be incubated in the presence of 80 to 120 ng/ml of IGF-1, 0.1 to 0.4 μM A83-01, 0.1 to 0.4 μM dorsopmorphin, and 0.1 to 0.4 μM PNU-74654. As another example, example, pluripotent stem cells can be incubated in the presence of 100 ng/ml of IGF-1, 0.2 μM A83-01, 0.2 μM dorsopmorphin, and 0.2 μM PNU-74654. Other factors can be used, for example, in combination with, or instead of, these factors and at similar concentrations.

Also disclosed are methods of producing neural cells, the method comprising (a) culturing pluripotent stem cells in the presence of fibroblast growth factor 2 (FGF-2); (b) incubating the stem cells in the presence of an amount of one or more Neural Development Factors (NDF) sufficient to generate cells expressing Pax-6, Otx2, Nestin, or a combination, thereby producing neural cells, wherein the NDF activate the phosphatidylinositol 3-kinase signaling pathway, inhibit the TGF-β superfamily signaling pathway, and inhibit the Wnt signaling pathway, wherein the NDF comprise Midkine, Pleiotrophin, insulin-like growth factor-1, or a combination; A83-01, SB431542, or a combination; dorsopmorphin; and PNU-74654, Dickkopf, or a combination, wherein prior to incubating in the presence of the NDF, at the same time as incubating in the presence of the NDF is initiated, or during incubating in the presence the NDF the use of FGF-2 is discontinued; and (c) culturing the neural cells, wherein the neural cells are cultured on a treated polymer substrate, wherein the neural cells are cultured in serum free conditions, wherein the neural cells are cultured in the presence of fibroblast growth factor 2 (FGF-2). The treated polymer substrate can be, for example, CELLBIND™, or substrate treated with Matrigel™, Geltrex™, or fibronectin. The treated polymer substrate can comprise, for example, CELLBIND™, or substrate treated with Matrigel™, Geltrex™, or fibronectin. The serum free conditions can comprise N2 supplement and B27 supplement. The neural cells can be cultured in the presence of epidermal growth factor (EGF). The neural cells can be cryopreservable. The stem can also be cultured in the presence of conditioned media or chemically defined media such as mouse embryonic fibroblast-conditioned media (MEF-CM), mTeSR™ or StemPro™. The stem cells can also be incubated in the presence of 20% knockout serum replacement (KSR).

Also disclosed are methods of producing neural cells, the method comprising (a) culturing pluripotent stem cells in the presence of fibroblast growth factor 2 (FGF-2); (b) incubating the stem cells in the presence of an amount of one or more Neural Development Factors (NDF) sufficient to generate cells expressing Pax-6, Otx2, Nestin, or a combination, thereby producing neural cells, wherein the NDF activate the phosphatidylinositol 3-kinase signaling pathway, activate the MAPK signaling pathway, inhibit the TGF-β superfamily signaling pathway, and inhibit the Wnt signaling pathway, wherein the NDF comprise Midkine, Pleiotrophin, insulin-like growth factor-1, or a combination; A83-01, SB431542, or a combination; dorsopmorphin; and PNU-74654, Dickkopf, or a combination, wherein prior to incubating in the presence of the NDF, at the same time as incubating in the presence of the NDF is initiated, or during incubating in the presence the NDF the use of FGF-2 is discontinued; and (c) culturing the neural cells, wherein the neural cells are cultured on a treated polymer substrate, wherein the neural cells are cultured in serum free conditions, wherein the neural cells are cultured in the presence of fibroblast growth factor 2 (FGF-2).

Also disclosed are methods of producing neural cells, the method comprising (a) incubating pluripotent stem cells in the presence of an amount of one or more Neural Development Factors (NDF) sufficient to generate cells expressing Pax-6, Otx2, Nestin, or a combination, thereby producing neural cells, wherein the NDF activate the phosphatidylinositol 3-kinase signaling pathway, activate the MAPK signaling pathway, inhibit the TGF-β superfamily signaling pathway, and inhibit the Wnt signaling pathway, wherein the NDF comprise Midkine, Pleiotrophin, insulin-like growth factor-1, or a combination; A83-01, SB431542, or a combination; dorsopmorphin; and PNU-74654, Dickkopf, or a combination; and (b) culturing the neural cells, wherein the neural cells are cultured on a treated polymer substrate, wherein the neural cells are cultured in serum free conditions, wherein the neural cells are cultured in the presence of fibroblast growth factor 2 (FGF-2).

Also disclosed are methods of producing neural cells, the method comprising culturing neural stem cells, wherein the neural stem cells are cultured on a treated polymer substrate, wherein the neural stem cells are cultured in serum free conditions, wherein the neural stem cells are cultured in the presence of fibroblast growth factor 2 (FGF-2), wherein the neural stem cells were produced by incubating pluripotent stem cells in the presence of an amount of one or more Neural Development Factors (NDF) sufficient to generate cells expressing Pax-6, Otx2, Nestin, or a combination, thereby producing the neural stem cells, wherein the NDF activate the phosphatidylinositol 3-kinase signaling pathway, activate the MAPK signaling pathway, inhibit the TGF-β superfamily signaling pathway, and inhibit the Wnt signaling pathway, wherein the NDF comprise Midkine, Pleiotrophin, insulin-like growth factor-1, or a combination; A83-01, SB431542, or a combination; dorsopmorphin; and PNU-74654, Dickkopf, or a combination.

Also disclosed are methods of producing neural cells, the method comprising incubating pluripotent stem cells in the presence of an amount of one or more Neural Development Factors (NDF) sufficient to generate cells expressing Pax-6, Otx2, Nestin, or a combination, thereby producing neural cells, wherein the NDF activate the phosphatidylinositol 3-kinase signaling pathway, inhibit the TGF-β superfamily signaling pathway, and inhibit the Wnt signaling pathway, wherein the NDF comprise Midkine, Pleiotrophin, insulin-like growth factor-1, or a combination; A83-01, SB431542, or a combination; dorsopmorphin; and PNU-74654, Dickkopf, or a combination.

Also disclosed are methods of producing neural cells, the method comprising incubating pluripotent stem cells in the presence of an amount of one or more Neural Development Factors (NDF) sufficient to generate cells expressing Pax-6, Otx2, Nestin, or a combination, thereby producing neural cells, wherein the NDF activate the phosphatidylinositol 3-kinase signaling pathway, activate the MAPK signaling pathway, inhibit the TGF-β superfamily signaling pathway, and inhibit the Wnt signaling pathway, wherein the NDF comprise Midkine, Pleiotrophin, insulin-like growth factor-1, or a combination; A83-01, SB431542, or a combination; dorsopmorphin; and PNU-74654, Dickkopf, or a combination.

Also disclosed are methods of producing neural cells, the method comprising (a) culturing pluripotent stem cells in the presence of fibroblast growth factor 2 (FGF-2); (b) incubating the stem cells in the presence of an amount of one or more Neural Development Factors (NDF) sufficient to generate cells expressing Pax-6, Otx2, Nestin, or a combination, thereby producing neural cells, wherein the NDF activate the phosphatidylinositol 3-kinase signaling pathway, inhibit the TGF-β superfamily signaling pathway, and inhibit the Wnt signaling pathway, wherein the NDF comprise Midkine, Pleiotrophin, insulin-like growth factor-1, or a combination; A83-01, SB431542, or a combination; dorsopmorphin; and PNU-74654, Dickkopf, or a combination, wherein prior to incubating in the presence of the NDF, at the same time as incubating in the presence of the NDF is initiated, or during incubating in the presence the NDF the use of FGF-2 is discontinued; and (c) culturing the neural cells, wherein the neural cells are cultured on a treated polymer substrate, wherein the neural cells are cultured in serum free conditions, wherein the neural cells are cultured in the presence of fibroblast growth factor 2 (FGF-2).

Also disclosed are methods of producing neural cells, the method comprising (a) culturing pluripotent stem cells in the presence of fibroblast growth factor 2 (FGF-2); (b) incubating the stem cells in the presence of an amount of one or more Neural Development Factors (NDF) sufficient to generate cells expressing Pax-6, Otx2, Nestin, or a combination, thereby producing neural cells, wherein the NDF activate the phosphatidylinositol 3-kinase signaling pathway, activate the MAPK signaling pathway, inhibit the TGF-β superfamily signaling pathway, and inhibit the Wnt signaling pathway, wherein the NDF comprise Midkine, Pleiotrophin, insulin-like growth factor-1, or a combination; A83-01, SB431542, or a combination; dorsopmorphin; and PNU-74654, Dickkopf, or a combination, wherein prior to incubating in the presence of the NDF, at the same time as incubating in the presence of the NDF is initiated, or during incubating in the presence the NDF the use of FGF-2 is discontinued; and (c) culturing the neural cells, wherein the neural cells are cultured on a treated polymer substrate, wherein the neural cells are cultured in serum free conditions, wherein the neural cells are cultured in the presence of fibroblast growth factor 2 (FGF-2).

For example, pluripotent stem cells (of any type and form any source) can be cultured in the presence of fibroblast growth factor 2 (FGF-2). For this purpose, FGF-2 can be used at a concentration of, for example, 40 to 150 ng/ml, 80 to 120 ng/ml, or 100 ng/ml. The cultured stem cells can then be incubated in the presence of Neural Development Factors (NDF) Midkine, Pleiotrophin, insulin-like growth factor-1, or a combination; A83-01, SB431542, or a combination; dorsopmorphin; and PNU-74654, Dickkopf, or a combination The NDF should be used at concentrations sufficient to activate the phosphatidylinositol 3-kinase signaling pathway, activate the MAPK signaling pathway, inhibit the TGF-β superfamily signaling pathway, and inhibit the Wnt signaling pathway. For this purpose, the Midkine, Pleiotrophin, and insulin-like growth factor-1 can be used at concentrations of, for example, 40 to 150 ng/ml, 80 to 120 ng/ml, or 100 ng/ml; and A83-01, SB431542, dorsopmorphin, PNU-74654, and Dickkopf can be used at concentrations of, for example, 0.05 to 2 μM, 0.1 to 0.4 μM, or 0.2 μM. The result should be the generation of cells expressing Pax-6, Otx2, Nestin, or a combination, which are markers of neural cells. Prior to incubating in the presence of the NDF, at the same time as incubating in the presence of the NDF is initiated, or during incubating in the presence the NDF the use of FGF-2 should be discontinued. The resulting neural cells can then be cultured. For example, the neural cells can be cultured on a treated polymer substrate, in serum free conditions, and in the presence of FGF-2. For this purpose, FGF-2 can be used at a concentration of, for example, 40 to 150 ng/ml, 80 to 120 ng/ml, or 100 ng/ml.

The treated polymer substrate can be, for example, CELLBIND™, or substrate treated with Matrigel™, Geltrex™, or fibronectin. The serum free conditions can comprise N2 supplement and B27 supplement. The neural cells can be cultured in the presence of epidermal growth factor (EGF). The neural cells can be cryopreservable. The stem can also be cultured in the presence of conditioned media or chemically defined media such as mouse embryonic fibroblast-conditioned media (MEF-CM), mTeSR™ or StemPro™. The stem cells can also be incubated in the presence of 20% knockout serum replacement (KSR).

1. Pre-Incubation

The disclosed method of producing neural cells uses stem cells, such as pluripotent stem cells. The stem cells can be pre-incubated prior to neural induction. The methods can be performed wherein prior to incubating in the presence of the NDF, the stem cells are cultured in the absence of feeder cells and on an extracellular matrix. The extracellular matrix can be, for example, Matrigel™ or Geltrex™. The methods can be performed wherein prior to incubating in the presence of the NDF the stem cells are cultured on fibroblasts. The fibroblasts can be, for example, from the same species as the stem cells. The fibroblasts can be, for example, from the same subject as the stem cells. The fibroblasts can be, for example, human fibroblasts.

The methods can be performed wherein prior to incubating in the presence of the NDF, the stem cells are cultured in the presence of fibroblast growth factor 2 (FGF-2). The stem can also be cultured in the presence of conditioned media or chemically defined media such as mouse embryonic fibroblast-conditioned media (MEF-CM), mTeSR™ or StemPro™. The methods can be performed wherein prior to incubating in the presence of the NDF, at the same time as incubating in the presence of the NDF is initiated, or during incubating in the presence the NDF the use of FGF-2 is discontinued. The methods can be performed wherein the use of FGF-2 is discontinued at the same time as incubating in the presence of the NDF is initiated. The methods can be performed wherein the use of FGF-2 is discontinued at the same time as incubating in the presence of the NDF is initiated by replacing growth medium containing FGF-2 and lacking NDF with growth medium lacking FGF-2 and containing the NDF. Use of conditioned media can be discontinued when the use of FGF-2 is discontinued. The methods can be performed wherein prior to incubating in the presence of the NDF, at least a portion of the stem cells are cultured to a density of 1×10³, 1×10⁴, 1×10⁵ cells per square centimeter or greater and/or to a density of at least 1×10³, 1×10⁴, 1×10⁵ cells per square centimeter.

2. Incubation

Disclosed are methods for producing neural cells from stem cells by incubating the stem cells under conditions disclosed herein. For example, disclosed are methods of producing neural cells, the method comprising incubating pluripotent stem cells in the presence of an amount of one or more Neural Development Factors (NDF) sufficient to generate cells expressing Pax-6, Otx2, Nestin, or a combination, thereby producing neural cells.

Also disclosed are methods of producing neural cells, the method comprising incubating pluripotent stem cells in the presence of an amount of one or more Neural Development Factors (NDF) sufficient to generate cells expressing Pax-6, Otx2, Nestin, or a combination, thereby producing neural cells, wherein the NDF activate the phosphatidylinositol 3-kinase signaling pathway, inhibit the TGF-β superfamily signaling pathway, and inhibit the Wnt signaling pathway, wherein the NDF comprise Midkine, Pleiotrophin, insulin-like growth factor-1, or a combination; A83-01, SB431542, or a combination; dorsopmorphin; and PNU-74654, Dickkopf, or a combination.

Also disclosed are methods of producing neural cells, the method comprising (a) culturing pluripotent stem cells in the presence of fibroblast growth factor 2 (FGF-2); (b) incubating the stem cells in the presence of an amount of one or more Neural Development Factors (NDF) sufficient to generate cells expressing Pax-6, Otx2, Nestin, or a combination, thereby producing neural cells, wherein the NDF activate the phosphatidylinositol 3-kinase signaling pathway, inhibit the TGF-β superfamily signaling pathway, and inhibit the Wnt signaling pathway, wherein the NDF comprise Midkine, Pleiotrophin, insulin-like growth factor-1, or a combination; A83-01, SB431542, or a combination; dorsopmorphin; and PNU-74654, Dickkopf, or a combination, wherein prior to incubating in the presence of the NDF, at the same time as incubating in the presence of the NDF is initiated, or during incubating in the presence the NDF the use of FGF-2 is discontinued; and (c) culturing the neural cells, wherein the neural cells are cultured on a treated polymer substrate, wherein the neural cells are cultured in serum free conditions, wherein the neural cells are cultured in the presence of fibroblast growth factor 2 (FGF-2). The treated polymer substrate can be, for example, CELLBIND™, or substrate treated with Matrigel™, Geltrex™, or fibronectin. The treated polymer substrate can comprise, for example, CELLBIND™, or substrate treated with Matrigel™, Geltrex™, or fibronectin. The serum free conditions can comprise N2 supplement and B27 supplement. The neural cells can be cultured in the presence of epidermal growth factor (EGF). The neural cells can be cryopreservable. The stem can also be cultured in the presence of conditioned media or chemically defined media such as mouse embryonic fibroblast-conditioned media (MEF-CM), mTeSR™ or StemPro™. The stem cells can also be incubated in the presence of 20% knockout serum replacement (KSR).

Also disclosed are methods of producing neural cells, the method comprising incubating pluripotent stem cells in the presence of an amount of one or more Neural Development Factors (NDF) sufficient to generate cells expressing Pax-6, Otx2, Nestin, or a combination, thereby producing neural cells, wherein the NDF activate the phosphatidylinositol 3-kinase signaling pathway, inhibit the TGF-β superfamily signaling pathway, and inhibit the Wnt signaling pathway, wherein the NDF comprise Midkine, Pleiotrophin, insulin-like growth factor-1, or a combination; A83-01, SB431542, or a combination; dorsopmorphin; and PNU-74654, Dickkopf, or a combination.

Also disclosed are methods of producing neural cells, the method comprising incubating pluripotent stem cells in the presence of an amount of one or more Neural Development Factors (NDF) sufficient to generate cells expressing Pax-6, Otx2, Nestin, or a combination, thereby producing neural cells, wherein the NDF activate the phosphatidylinositol 3-kinase signaling pathway, activate the Notch signaling pathway, inhibit the TGF-β superfamily signaling pathway, and inhibit the Wnt signaling pathway, wherein the NDF comprise Midkine, Pleiotrophin, insulin-like growth factor-1, or a combination; Delta-1, Delta-2, Delta-3, Delta-4, Jagged-1, Jagged-2, or a combination; A83-01, SB431542, or a combination; dorsopmorphin; and PNU-74654, Dickkopf, or a combination.

Also disclosed are methods of producing neural cells, the method comprising (a) culturing pluripotent stem cells in the presence of fibroblast growth factor 2 (FGF-2); (b) incubating the stem cells in the presence of an amount of one or more Neural Development Factors (NDF) sufficient to generate cells expressing Pax-6, Otx2, Nestin, or a combination, thereby producing neural cells, wherein the NDF activate the phosphatidylinositol 3-kinase signaling pathway, inhibit the TGF-β superfamily signaling pathway, and inhibit the Wnt signaling pathway, wherein the NDF comprise Midkine, Pleiotrophin, insulin-like growth factor-1, or a combination; A83-01, SB431542, or a combination; dorsopmorphin; and PNU-74654, Dickkopf, or a combination, wherein prior to incubating in the presence of the NDF, at the same time as incubating in the presence of the NDF is initiated, or during incubating in the presence the NDF the use of FGF-2 is discontinued; and (c) culturing the neural cells, wherein the neural cells are cultured on a treated polymer substrate, wherein the neural cells are cultured in serum free conditions, wherein the neural cells are cultured in the presence of fibroblast growth factor 2.

Also disclosed are methods of producing neural cells, the method comprising (a) culturing pluripotent stem cells in the presence of fibroblast growth factor 2 (FGF-2); (b) incubating the stem cells in the presence of an amount of one or more Neural Development Factors (NDF) sufficient to generate cells expressing Pax-6, Otx2, Nestin, or a combination, thereby producing neural cells, wherein the NDF activate the phosphatidylinositol 3-kinase signaling pathway, activate the Notch signaling pathway, inhibit the TGF-β superfamily signaling pathway, and inhibit the Wnt signaling pathway, wherein the NDF comprise Midkine, Pleiotrophin, insulin-like growth factor-1, or a combination; Delta-1, Delta-2, Delta-3, Delta-4, Jagged-1, Jagged-2, or a combination; A83-01, SB431542, or a combination; dorsopmorphin; and PNU-74654, Dickkopf, or a combination, wherein prior to incubating in the presence of the NDF, at the same time as incubating in the presence of the NDF is initiated, or during incubating in the presence the NDF the use of FGF-2 is discontinued; and (c) culturing the neural cells, wherein the neural cells are cultured on a treated polymer substrate, wherein the neural cells are cultured in serum free conditions, wherein the neural cells are cultured in the presence of fibroblast growth factor 2 (FGF-2).

The treated polymer substrate can be, for example, CELLBIND™, or substrate treated with Matrigel™, Geltrex™, or fibronectin. The serum free conditions can comprise N2 supplement and B27 supplement. The neural cells can be cultured in the presence of epidermal growth factor (EGF). The neural cells can be cryopreservable. The stem can also be cultured in the presence of conditioned media or chemically defined media such as mouse embryonic fibroblast-conditioned media (MEF-CM), mTeSR™ or StemPro™. The stem cells can also be incubated in the presence of 20% knockout serum replacement (KSR).

The disclosed methods for producing neural cells from stem cells can involve incubation with NDF. The NDF can, for example, activate the phosphatidylinositol 3-kinase (PI3K) signaling pathway. The NDF can, for example, activate the mitogen-activated protein kinase (MAPK) signaling pathway. The NDF can, for example, activate the phosphatidylinositol 3-kinase signaling pathway and can activate the MAPK signaling pathway. The NDF can, for example, activate the phosphatidylinositol 3-kinase signaling pathway and the MAPK signaling pathway. The NDF can, for example, activate the phosphatidylinositol 3-kinase signaling pathway and the MAPK signaling pathway in a balanced manner. The NDF can, for example, inhibit the TGF-β superfamily signaling pathway. The NDF can, for example, inhibit the Wnt signaling pathway. The NDF can, for example, activate the phosphatidylinositol 3-kinase signaling pathway, inhibit the TGF-β superfamily signaling pathway, and inhibit the Wnt signaling pathway. The NDF can, for example, activate the phosphatidylinositol 3-kinase signaling pathway, inhibit the TGF-β superfamily signaling pathway, inhibit the Wnt signaling pathway, or any combination of these. The NDF can, for example, activate the phosphatidylinositol 3-kinase signaling pathway, activate the MAPK signaling pathway, inhibit the TGF-β superfamily signaling pathway, and inhibit the Wnt signaling pathway. The Wnt signaling pathway can be, for example, inhibited after the β-catenin destruction complex. The NDF can, for example, activate the Notch signaling pathway. The NDF can, for example, activate the phosphatidylinositol 3-kinase signaling pathway, activate the Notch signaling pathway, inhibit the TGF-β superfamily signaling pathway, and inhibit the Wnt signaling pathway. The NDF can, for example, activate the phosphatidylinositol 3-kinase signaling pathway, activate the Notch signaling pathway, inhibit the TGF-β superfamily signaling pathway, inhibit the Wnt signaling pathway, or any combination of these.

The NDF can comprise, for example, an activator of the phosphatidylinositol 3-kinase signaling pathway. The NDF can comprise, for example, an activator of the MAPK signaling pathway. The NDF can comprise, for example, an activator of the phosphatidylinositol 3-kinase signaling pathway and the MAPK signaling pathway. The NDF can comprise, for example, Midkine, Pleiotrophin, insulin-like growth factor-1, or a combination. The NDF can comprise, for example, Midkine, insulin-like growth factor-1, or a combination. The NDF can comprise, for example, Midkine. The NDF can comprise, for example, an activator of the phosphatidylinositol 3-kinase signaling pathway and an activator of the MAPK signaling pathway. The NDF can comprise, for example, an inhibitor of the TGF-β superfamily signaling pathway. The NDF can comprise, for example, A83-01, SB431542, or a combination. The NDF can comprise, for example, dorsomorphin. The NDF can comprise, for example, dorsomorphin and A83-01. The NDF can comprise, for example, an inhibitor of the Wnt signaling pathway. The NDF can comprise, for example, PNU-74654, Dickkopf, or a combination. The NDF can comprise, for example, an activator of the phosphatidylinositol 3-kinase signaling pathway, an inhibitor of the TGF-β superfamily signaling pathway, and an inhibitor of the Wnt signaling pathway. The NDF can comprise, for example, an activator of the phosphatidylinositol 3-kinase signaling pathway, an inhibitor of the TGF-β superfamily signaling pathway, an inhibitor of the Wnt signaling pathway, or any combination of these. The NDF can comprise, for example, Midkine, Pleiotrophin, insulin-like growth factor-1, or a combination; A83-01, SB431542, or a combination; dorsopmorphin; and PNU-74654, Dickkopf, or a combination. The NDF can comprise, for example, Midkine, Pleiotrophin, insulin-like growth factor-1, or a combination; A83-01, SB431542, or a combination; dorsopmorphin; PNU-74654, Dickkopf, or a combination; or any combination of these. The NDF can comprise, for example, Midkine, A83-01, dorsopmorphin, and PNU-74654. The NDF can comprise, for example, insulin-like growth factor-1, A83-01, dorsopmorphin, and PNU-74654.

The NDF can comprise, for example, an activator of the Notch signaling pathway. The NDF can comprise, for example, Delta-1, Delta-2, Delta-3, Delta-4, Jagged-1, Jagged-2, or a combination. The NDF can comprise, for example, an activator of the phosphatidylinositol 3-kinase signaling pathway, an activator of the Notch signaling pathway, an inhibitor of the TGF-β superfamily signaling pathway, and an inhibitor of the Wnt signaling pathway. The NDF can comprise, for example, an activator of the phosphatidylinositol 3-kinase signaling pathway, an activator of the Notch signaling pathway, an inhibitor of the TGF-β superfamily signaling pathway, an inhibitor of the Wnt signaling pathway, or any combination of these. The NDF can comprise, for example, Midkine, Pleiotrophin, insulin-like growth factor-1, or a combination; Delta-1, Delta-2, Delta-3, Delta-4, Jagged-1, Jagged-2, or a combination; A83-01, SB431542, or a combination; dorsopmorphin; and PNU-74654, Dickkopf, or a combination. The NDF can comprise, for example, Midkine, Pleiotrophin, insulin-like growth factor-1, or a combination; Delta-1, Delta-2, Delta-3, Delta-4, Jagged-1, Jagged-2, or a combination; A83-01, SB431542, or a combination; dorsopmorphin; PNU-74654, Dickkopf, or a combination; or any combination of these.

The NDF can consist of, for example, an activator of the phosphatidylinositol 3-kinase signaling pathway. The NDF can consist of, for example, an activator of the MAPK signaling pathway. The NDF can consist of, for example, an activator of the phosphatidylinositol 3-kinase signaling pathway and the MAPK signaling pathway. The NDF can consist of, for example, Midkine, Pleiotrophin, insulin-like growth factor-1, or a combination. The NDF can consist of, for example, Midkine, insulin-like growth factor-1, or a combination. The NDF can consist of, for example, Midkine. The NDF can consist of, for example, an activator of the phosphatidylinositol 3-kinase signaling pathway and an activator of the MAPK signaling pathway. The NDF can consist of, for example, an inhibitor of the TGF-β superfamily signaling pathway. The NDF can consist of, for example, A83-01, SB431542, or a combination. The NDF can consist of, for example, dorsomorphin. The NDF can consist of, for example, dorsomorphin and A83-01. The NDF can consist of, for example, an inhibitor of the Wnt signaling pathway. The NDF can consist of, for example, PNU-74654, Dickkopf, or a combination. The NDF can consist of, for example, an activator of the phosphatidylinositol 3-kinase signaling pathway, an inhibitor of the TGF-β superfamily signaling pathway, and an inhibitor of the Wnt signaling pathway. The NDF can consist of, for example, an activator of the phosphatidylinositol 3-kinase signaling pathway, an inhibitor of the TGF-β superfamily signaling pathway, an inhibitor of the Wnt signaling pathway, or any combination of these. The NDF can consist of, for example, Midkine, Pleiotrophin, insulin-like growth factor-1, or a combination; A83-01, SB431542, or a combination; dorsopmorphin; and PNU-74654, Dickkopf, or a combination. The NDF can consist of, for example, Midkine, Pleiotrophin, insulin-like growth factor-1, or a combination; A83-01, SB431542, or a combination; dorsopmorphin; PNU-74654, Dickkopf, or a combination; or any combination of these. The NDF can consist of, for example, Midkine, A83-01, dorsopmorphin, and PNU-74654. The NDF can consist of, for example, insulin-like growth factor-1, A83-01, dorsopmorphin, and PNU-74654.

The NDF can consist of, for example, an activator of the Notch signaling pathway. The NDF can consist of, for example, Delta-1, Delta-2, Delta-3, Delta-4, Jagged-1, Jagged-2, or a combination. The NDF can consist of, for example, an activator of the phosphatidylinositol 3-kinase signaling pathway, an activator of the Notch signaling pathway, an inhibitor of the TGF-β superfamily signaling pathway, and an inhibitor of the Wnt signaling pathway. The NDF can consist of, for example, an activator of the phosphatidylinositol 3-kinase signaling pathway, an activator of the Notch signaling pathway, an inhibitor of the TGF-β superfamily signaling pathway, an inhibitor of the Wnt signaling pathway, or any combination of these. The NDF can consist of, for example, Midkine, Pleiotrophin, insulin-like growth factor-1, or a combination; Delta-1, Delta-2, Delta-3, Delta-4, Jagged-1, Jagged-2, or a combination; A83-01, SB431542, or a combination; dorsopmorphin; and PNU-74654, Dickkopf, or a combination. The NDF can consist of, for example, Midkine, Pleiotrophin, insulin-like growth factor-1, or a combination; Delta-1, Delta-2, Delta-3, Delta-4, Jagged-1, Jagged-2, or a combination; A83-01, SB431542, or a combination; dorsopmorphin; PNU-74654, Dickkopf, or a combination; or any combination of these.

The NDF can consist essentially of, for example, an activator of the phosphatidylinositol 3-kinase signaling pathway. The NDF can consist essentially of, for example, an activator of the MAPK signaling pathway. The NDF can consist essentially of, for example, an activator of the phosphatidylinositol 3-kinase signaling pathway and the MAPK signaling pathway. The NDF can consist essentially of, for example, Midkine, Pleiotrophin, insulin-like growth factor-1, or a combination. The NDF can consist essentially of, for example, Midkine, insulin-like growth factor-1, or a combination. The NDF can consist essentially of, for example, Midkine. The NDF can consist essentially of, for example, an activator of the phosphatidylinositol 3-kinase signaling pathway and an activator of the MAPK signaling pathway. The NDF can consist essentially of, for example, an inhibitor of the TGF-β superfamily signaling pathway. The NDF can consist essentially of, for example, A83-01, SB431542, or a combination. The NDF can consist essentially of, for example, dorsomorphin. The NDF can consist essentially of, for example, dorsomorphin and A83-01. The NDF can consist essentially of, for example, an inhibitor of the Wnt signaling pathway. The NDF can consist essentially of, for example, PNU-74654, Dickkopf, or a combination. The NDF can consist essentially of, for example, an activator of the phosphatidylinositol 3-kinase signaling pathway, an inhibitor of the TGF-β superfamily signaling pathway, and an inhibitor of the Wnt signaling pathway. The NDF can consist essentially of, for example, an activator of the phosphatidylinositol 3-kinase signaling pathway, an inhibitor of the TGF-β superfamily signaling pathway, an inhibitor of the Wnt signaling pathway, or any combination of these. The NDF can consist essentially of, for example, Midkine, Pleiotrophin, insulin-like growth factor-1, or a combination; Delta-1, Delta-2, Delta-3, Delta-4, Jagged-1, Jagged-2, or a combination; A83-01, SB431542, or a combination; dorsopmorphin; and PNU-74654, Dickkopf, or a combination. The NDF can consist essentially of, for example, Midkine, Pleiotrophin, insulin-like growth factor-1, or a combination; Delta-1, Delta-2, Delta-3, Delta-4, Jagged-1, Jagged-2, or a combination; A83-01, SB431542, or a combination; dorsopmorphin; PNU-74654, Dickkopf, or a combination; or any combination of these. The NDF can consist essentially of, for example, Midkine, A83-01, dorsopmorphin, and PNU-74654. The NDF can consist essentially of, for example, insulin-like growth factor-1, A83-01, dorsopmorphin, and PNU-74654.

The NDF can consist essentially of, for example, an activator of the Notch signaling pathway. The NDF can consist essentially of, for example, Delta-1, Delta-2, Delta-3, Delta-4, Jagged-1, Jagged-2, or a combination. The NDF can consist essentially of, for example, an activator of the phosphatidylinositol 3-kinase signaling pathway, an activator of the Notch signaling pathway, an inhibitor of the TGF-β superfamily signaling pathway, and an inhibitor of the Wnt signaling pathway. The NDF can consist essentially of, for example, an activator of the phosphatidylinositol 3-kinase signaling pathway, an activator of the Notch signaling pathway, an inhibitor of the TGF-β superfamily signaling pathway, an inhibitor of the Wnt signaling pathway, or any combination of these. The NDF can consist essentially of, for example, Midkine, Pleiotrophin, insulin-like growth factor-1, or a combination; Delta-1, Delta-2, Delta-3, Delta-4, Jagged-1, Jagged-2, or a combination; A83-01, SB431542, or a combination; dorsopmorphin; and PNU-74654, Dickkopf, or a combination. The NDF can consist essentially of, for example, Midkine, Pleiotrophin, insulin-like growth factor-1, or a combination; Delta-1, Delta-2, Delta-3, Delta-4, Jagged-1, Jagged-2, or a combination; A83-01, SB431542, or a combination; dorsopmorphin; PNU-74654, Dickkopf, or a combination; or any combination of these.

The NDF can, for example, activate the Notch signaling pathway. The NDF can comprise, for example, Delta-1, Delta-2, Delta-3, Delta-4, Jagged-1, Jagged-2, or a combination. The NDF can, for example, activate the protein kinase signaling pathway. The NDF can comprise, for example, Forskolin, dibutyryl cAMP, or a combination. The NDF can, for example, activate tyrosine kinase anaplastic lymphoma kinase (ALK). The NDF can, for example, activate insulin-like growth factor (IGF) receptor. The NDF can, for example, activate phosphatidylinositol 3-kinase. The NDF can, for example, inhibit Activin receptor-like kinase 5 (ALK5). The NDF can, for example, inhibit Activin receptor-like kinase 4 (ALK4). The NDF can, for example, inhibit Activin receptor-like kinase 7 (ALK7). The NDF can, for example, inhibit ALK5, ALK4, and ALK7. The NDF can, for example, inhibit protein phosphatase 2A (PP2A). The NDF can, for example, inhibit adenosine monophosphate-activated protein kinase (AMPK). The NDF can, for example, inhibit Bone morphogenic protein (BMP) receptor. The NDF can, for example, inhibit interaction between β-catenin and T cell factor (TCF). The NDF can, for example, activate protein-tyrosine phosphatasζ (PTPζ). The NDF can, for example, inhibit SMAD2, SMAD3, SMAD4, or a combination. The NDF can, for example, activate Notch. The NDF can, for example, inhibit SMAD1, SMAD5, SMAD8, or a combination. The NDF can, for example, inhibit Wnt binding to Frizzled. The NDF can, for example, inhibit lipoprotein receptor-related protein (LRP) binding to Frizzled. The NDF can, for example, inhibit β-catenin stabilization. The NDF can, for example, inhibit β-catenin binding to T cell factor (TCT). The NDF can, for example, activate insulin-like growth factor-1 receptor (IGF-1R). The NDF can, for example, activate insulin receptor substrate-1 (IRS-1).

3. Culturing

The disclosed neural stem cells can be cultured. Thus, the disclosed methods can be performed further comprising culturing and multiplying the neural cells. The neural cells can be, for example, cultured on a treated polymer substrate. The treated polymer substrate can be, for example, CELLBIND™, or substrate treated with Matrigel™, Geltrex™, or fibronectin. The treated polymer substrate can comprise, for example, CELLBIND™, or substrate treated with Matrigel™, Geltrex™, or fibronectin. The neural cells can be, for example, cultured in serum free conditions. The serum free conditions can comprise N2 supplement and B27 supplement. The neural cells can be, for example, cultured in the presence of fibroblast growth factor 2 (FGF-2). The neural cells can be, for example, passaged with Accutase or collagenase IV. The stem cells can be, for example, human stem cells. The stem cells can be, for example, embryonic stem cells (ESC). The stem cells can be, for example, derived from embryonic or fetal tissue. The stem cells can be, for example, derived from postfetal tissue. The stem cells can be, for example, derived from adult tissue. The stem cells can be, for example, derived from adult tissue. The stem cells can be, for example, induced pluripotent stem cells (iPSC). The stem cells can be, for example, derived from a subject in need of neural cells. Neural stem cells obtain from other sources and by other methods can also be cultured according to the disclosed method.

The neural stems cells can be cultured to achieve neural patterning. Neural stem cells, such as the neural stem cells produced by the disclosed methods, can be, for example, directly plated on special cell plates, such as CELLBIND™ (Corning), or regular dishes coated with, for example, Matrigel™, Geltrex™, or fibronectin, and patterned to precursors of, for example, dopamine neurons (with, for example, sonic hedgehog and fibroblast growth factor 8) or motor neurons (with, for example, retinoid acid and sonic hedgehog). Since the disclosed methods generate highly pure adherently growing neural stem cell cultures, no additional cell enrichment strategies are necessary. The cells can also be cultured on other treated polymer substrates such as or substrate treated with Matrigel™, Geltrex™, or fibronectin.

Disclosed are methods of culturing neural stem cells, the method comprising culturing neural stem cells, wherein the neural stem cells are cultured on a treated polymer substrate, wherein the neural stem cells are cultured in serum free conditions, wherein the neural stem cells are cultured in the presence of fibroblast growth factor 2 (FGF-2), wherein the neural stem cells were produced by incubating pluripotent stem cells in the presence of an amount of one or more Neural Development Factors (NDF) sufficient to generate cells expressing Pax-6, Otx2, Nestin, or a combination, thereby producing the neural stem cells, wherein the NDF activate the phosphatidylinositol 3-kinase signaling pathway, activate the MAPK signaling pathway, inhibit the TGF-β superfamily signaling pathway, and inhibit the Wnt signaling pathway, wherein the NDF comprise Midkine, Pleiotrophin, insulin-like growth factor-1, or a combination; A83-01, SB431542, or a combination; dorsopmorphin; and PNU-74654, Dickkopf, or a combination.

Disclosed are methods of using neural cells and neural stem cells produced in the disclosed methods. Also disclosed are methods of treating a subject using the neural cells and neural stem cells produced in the disclosed methods. Also disclosed are methods of treating a subject using the neural cells and neural stem cells produced in the disclosed methods where the neural cells were produced from pluripontent stem cells derived from the subject. Also disclosed are methods of detecting a state or characteristic of neural cells and neural stem cells produced in the disclosed methods. Also disclosed are methods testing conditions for differentiation of neural stem cells using the neural cells and neural stem cells produced in the disclosed methods.

Also disclosed are methods of treating a subject, the method comprising administering a neural cell produced by one or more of the disclosed methods. Also disclosed are methods of treating a subject, the method comprising incubating pluripotent stem cells in the presence of an amount of one or more Neural Development Factors (NDF) sufficient to generate cells expressing Pax-6, Otx2, Nestin, or a combination, thereby producing neural cells, and administering one or more of the neural cells to the subject. The stem cell can be, for example, from the same species as the subject. The stem cell can be, for example, from the subject.

Also disclosed are methods of detecting a state or characteristic of a cell, the method comprising detecting the state or characteristic in a neural cell produced by one or more of the disclosed methods. Also disclosed are methods of testing conditions for differentiation of neural stem cells, the method comprising exposing a neural cell produced by one or more of the disclosed methods to test conditions and determining if the neural stem cells differentiate into a cell type of interest. The cell type of interest can be, for example, neuron, astrocyte, oligodendrocyte, or a combination.

The disclosed neural stem cells can be used to prepare populations of differentiated neural cells of various commercially and therapeutically important tissue types. In general, this can be accomplished by expanding the neural stem cells to the desired number. Thereafter, they can be induced to differentiate according to any of a variety of differentiation strategies. For example, highly enriched populations of cells of the neural lineage can be generated by changing the cells to a culture medium containing one or more neurotrophins (such as neurotrophin 3, neurotrophin 4, brain-derived neurotrophic factor, or glial cell-derived factor), one or more mitogens (such as epidermal growth factor, FGF-2, PDGF, IGF 1, and erythropoietin), or one or more vitamins (such as retinoic acid, ascorbic acid). The disclosed neural stem cells have the capacity to generate both neuronal cells (including, for example, mature neurons) and glial cells (including, for example, radial glial cells expressing brain lipid-binding protein (BLBP), astrocytes, and oligodendrocytes, such as oligodendrocytes expressing the surface marker RIP).

As will be appreciated, differentiated cells derived from neural stem cells can be also be used for tissue reconstitution or regeneration in a subject, such as in a human patient, in need thereof. The cells can be administered in a manner that permits them to graft to the intended tissue site and reconstitute or regenerate the functionally deficient area. For instance, neural stem cells or neural precursor cells can be transplanted directly into parenchymal or intrathecal sites of the central nervous system, according to the disease being treated. The efficacy of neural cell transplants can be assessed in. for example, a rat model for acutely injured spinal cord, as described by McDonald, et al. ((1999) Nat. Med., vol. 5:1410) and Kim, et al. ((2002) Nature, vol. 418:50). Successful transplants will show transplant-derived cells present in the lesion 2-5 weeks later, differentiated into astrocytes, oligodendrocytes, and/or neurons, and migrating along the spinal cord from the lesioned end, and an improvement in gait, coordination, and weight-bearing.

For therapeutic application, cells prepared according to the disclosed methods (be they totipotent or pluripotent cells or differentiated cells derived therefrom) can typically be supplied in the form of a pharmaceutical composition comprising an isotonic excipient, and are prepared under conditions that are sufficiently sterile for human administration. For general principles in medicinal formulation of cell compositions, see “Cell Therapy: Stem Cell Transplantation, Gene Therapy, and Cellular Immunotherapy,” by Morstyn & Sheridan eds, Cambridge University Press, 1996; and “Hematopoietic Stem Cell Therapy,” E. D. Ball, J. Lister & P. Law, Churchill Livingstone, 2000. The cells can be packaged in a device or container suitable for distribution or clinical use, optionally accompanied by information relating to use of the cells in tissue regeneration or for restoring a therapeutically important metabolic function.

The disclosed methods can include assays for neural cell differentiation (for example, neurogenesis or neuronal differentiation), where production or generation of neurons or differentiation of neural stem cells is detected and/or quantified. The disclosed methods can comprise detecting and/or quantifying one or more neural cell-specific markers. The disclosed methods can comprise monitoring levels of neural cell differentiation for one or more particular neural cell sub-types or lineages, or levels of neural cell in general, depending on the markers selected. Generation of neural cells of defined lineages can be assayed, by, for example, detecting and/or quantifying lineage-specific markers. The disclosed methods can comprise, for example, contacting the cells with an antibody to a cell marker and determining binding, wherein the presence of the marker (and hence antibody binding) indicates that the cell is of a particular cell type, sub-type, lineage or sub-lineage. The disclosed methods can comprise, for example, determining or quantifying levels of antibody binding, and thereby determining or quantifying levels of differentiation, the stage of differentiation of the cells, and/or the percentage of cells of a particular type, sub-type, lineage or sub-lineage or at a particular stage of differentiation. Suitable markers are known to the person skilled in the art.

The disclosed methods for detecting neural cell differentiation are suitable for determining markers that can be used to identify stem cells, neural stem cells, and/or neural cells at particular stages of differentiation, or to identify the type or sub-type of the cell, and thus indicate the differentiation state of the cell or the cell type or sub-type. For example, assay methods can comprise inducing or allowing differentiation of stem cells to produce neural stem cells, and/or culturing neural stem cells to produce differentiated neural cells; comparing expression levels of proteins in cells at one stage of differentiation with expression levels of proteins in cells at a second stage of differentiation; and/or identifying proteins whose level of expression differs in cells at the first and second stages of differentiation. A difference in expression levels indicates that the protein may be used as a marker to indicate the differentiation state, type or sub-type of the cell and/or to distinguish cells at the first and second differentiation states. Expression levels can be compared using any appropriate method, which the skilled person can determine. Expression of proteins expressed at the cell surface can be compared, by, for example, contacting cells or a cell extract with a surface expression library of antibodies and determining binding. For example, the method can comprise comparing expression of proteins in neural stem cells with stem cells.

The difference in expression levels can be, for example, at least 1.2-fold, at least 1.5-fold, at least 1.6-fold, at least 1.8-fold, at least 2-fold, at least 3-fold, at least 5-fold, at least 10-fold, or more. Expression can be detected in cells at the first stage of differentiation and not detected at all in cells at a second stage of differentiation.

4. Differentiation

Disclosed are methods of producing differentiated neural cells. The method can comprise, for example, incubating pluripotent stem cells in the presence of an amount of one or more Neural Development Factors (NDF) sufficient to generate cells expressing Pax-6, Otx2, Nestin, or a combination, thereby producing neural cells, and differentiating the neural cells into differentiated neural cells. The neural cells can be differentiated into differentiated neural cells by, for example, incubating the neural cells under differentiation conditions. The differentiation conditions can be chosen based on the type of differentiated neural cells desired or sought. Also disclosed are methods for differentiation of neural stem cells, the method comprising exposing a neural cell produced by one or more of the disclosed methods to differentiation conditions. The differentiation conditions can produce a variety of differentiated neural cells or predominantely a single type or class if differentiated neural cell. A variety of differentiation conditions are known and can be used to produce differentiated neural cells. In addition, differentiation conditions identified in methods disclosed herein can be used to produce differentiated neural cells. The desired differentiated neural cell can be referred to a the cell type of interest. The cell type of interest can be, for example, neuron, astrocyte, oligodendrocyte, or a combination. The differentiated neural cells can comprise, for example, pyramidal neurons, such as cortical pyramidal neurons. The differentiated neural cells can comprise, for example, neurons of the ventral mesencephalon (substantia nigra). The differentiated neural cells can comprise, for example, dopamine neurons, neurons that express dopamine, neurons that express molecules required for dopamine synthesis, neurons that express molecules resulting from dopamine metabolism, or a combination. The differentiated neural cells can comprise, for example, motor neurons. The differentiated neural cells can comprise, for example, spinal ventral horn motor neurons. The neural cells can comprise, for example, glial cells, including, for example, radial glial cells expressing brain lipid-binding protein (BLBP). The neural cells can comprise, for example, retinal pigment epithelium expressing, for example, the transcription factors OTX2, microphthalmia-associated transcription factor (MITE), and the tight-junction protein ZO-1. The neural cells can comprise, for example, oligodendrocytes expressing, for example, the surface marker RIP (2′,3′-cyclic nucleotide 3′-phosphodiesterase (CNPase)). The neural cells can comprise, for example, interneurons. The differentiated neural cells can comprise, for example, GABA neurons, neurons that express GABA, neurons that express molecules required for GABA synthesis, neurons that express molecules resulting from GABA metabolism, or a combination. The differentiated neural cells can comprise, for example, glutamate neurons, neurons that express glutamate, neurons that express molecules required for glutamate synthesis, neurons that express molecules resulting from glutamate metabolism, or a combination.

The differentiated neural cells can comprise, for example, catecholaminergic neurons, neurons that express molecules required for synthesis of catecholaminergically active molecules, neurons that express molecules resulting from metabolism of catecholaminergically active molecules, or a combination. The differentiated neural cells can comprise, for example, serotoninergic neurons, neurons that express serotoninergically active molecules, neurons that express molecules required for synthesis of serotoninergically active molecules, neurons that express molecules resulting from metabolism of serotoninergically active molecules, or a combination. The differentiated neural cells can comprise, for example, cholinergic neurons, neurons that express cholinergically active molecules, neurons that express molecules required for synthesis of cholinergically active molecules, neurons that express molecules resulting from metabolism of cholinergically active molecules, or a combination.

The differentiated neural cells can be, for example, tyrosine hydroxylase+neurons, Oligo2+/HB9+ neurons, large glumatergic pyramidal-like neurons.

Disclosed are methods of producing differentiated neural cells, the method comprising inducing neural stem cells to differentiate into differentiated neural cells, wherein the differentiated neural stem cells are a desired type of differentiated neural cell, wherein the neural stem cells were produced by incubating pluripotent stem cells in the presence of an amount of one or more Neural Development Factors (NDF) sufficient to generate cells expressing Pax-6, Otx2, Nestin, or a combination, thereby producing the neural stem cells, wherein the NDF activate the phosphatidylinositol 3-kinase signaling pathway, activate the MAPK signaling pathway, inhibit the TGF-β superfamily signaling pathway, and inhibit the Wnt signaling pathway, wherein the NDF comprise Midkine, Pleiotrophin, insulin-like growth factor-1, or a combination; A83-01, SB431542, or a combination; dorsopmorphin; and PNU-74654, Dickkopf, or a combination.

### B. Treating

The disclosed methods and cells can be used in, as part of, or to produce materials for, treating subjects in need. For example, the disclosed neural cells and differentiated neural cells can be used to treat subjects and to provide compounds, compositions, and components for treating subjects. For example, the disclosed differentiated neural cells can be implanted in or administered to subjects in need of such cells. The pluripotent stem cells used in the disclosed methods can be from any source. Where the neural cells and differentiated neural cells are to be implanted in or administered to a subject, it is useful to use pluripotent stem cells from the subject to produce the neural cells and differentiated neural cells. For example, it is useful to use iPSCs from the subject to produce the neural cells and differentiated neural cells.

Disclosed are methods of treating a subject, the method comprising (a) producing induced pluripotent stem cells from a cell of the subject, (b) culturing the pluripotent stem cells in the presence of fibroblast growth factor 2 (FGF-2); (c) incubating the stem cells in the presence of an amount of one or more Neural Development Factors (NDF) sufficient to generate cells expressing Pax-6, Otx2, Nestin, or a combination, thereby producing neural cells, wherein the NDF activate the phosphatidylinositol 3-kinase signaling pathway, activate the MAPK signaling pathway, inhibit the TGF-β superfamily signaling pathway, and inhibit the Wnt signaling pathway, wherein the NDF comprise Midkine, Pleiotrophin, insulin-like growth factor-1, or a combination; A83-01, SB431542, or a combination; dorsopmorphin; and PNU-74654, Dickkopf, or a combination, wherein prior to incubating in the presence of the NDF, at the same time as incubating in the presence of the NDF is initiated, or during incubating in the presence the NDF the use of FGF-2 is discontinued; (d) culturing the neural cells, wherein the neural cells are cultured on a treated polymer substrate, wherein the neural cells are cultured in serum free conditions, wherein the neural cells are cultured in the presence of fibroblast growth factor 2 (FGF-2); (e) inducing the neural cells to differentiate into differentiated neural cells, wherein the differentiated neural cells are a desired type of differentiated neural cell; and (f) implanting in or administering to the subject the differentiated neural cells.

Also disclosed are methods of treating a subject, the method comprising (a) culturing pluripotent stem cells in the presence of fibroblast growth factor 2 (FGF-2); (b) incubating the stem cells in the presence of an amount of one or more Neural Development Factors (NDF) sufficient to generate cells expressing Pax-6, Otx2, Nestin, or a combination, thereby producing neural cells, wherein the NDF activate the phosphatidylinositol 3-kinase signaling pathway, activate the MAPK signaling pathway, inhibit the TGF-β superfamily signaling pathway, and inhibit the Wnt signaling pathway, wherein the NDF comprise Midkine, Pleiotrophin, insulin-like growth factor-1, or a combination; A83-01, SB431542, or a combination; dorsopmorphin; and PNU-74654, Dickkopf, or a combination, wherein prior to incubating in the presence of the NDF, at the same time as incubating in the presence of the NDF is initiated, or during incubating in the presence the NDF the use of FGF-2 is discontinued; (c) culturing the neural cells, wherein the neural cells are cultured on a treated polymer substrate, wherein the neural cells are cultured in serum free conditions, wherein the neural cells are cultured in the presence of fibroblast growth factor 2 (FGF-2); (d) inducing the neural cells to differentiate into differentiated neural cells, wherein the differentiated neural cells are a desired type of differentiated neural cell; and (e) implanting in or administering to the subject the differentiated neural cells. The pluripotent stem cells can be induced pluripotent stem cells produced from a cell of the subject.

Also disclosed are methods of treating a subject, the method comprising (a) incubating pluripotent stem cells in the presence of an amount of one or more Neural Development Factors (NDF) sufficient to generate cells expressing Pax-6, Otx2, Nestin, or a combination, thereby producing neural cells, wherein the NDF activate the phosphatidylinositol 3-kinase signaling pathway, activate the MAPK signaling pathway, inhibit the TGF-β superfamily signaling pathway, and inhibit the Wnt signaling pathway, wherein the NDF comprise Midkine, Pleiotrophin, insulin-like growth factor-1, or a combination; A83-01, SB431542, or a combination; dorsopmorphin; and PNU-74654, Dickkopf, or a combination; (b) culturing the neural cells, wherein the neural cells are cultured on a treated polymer substrate, wherein the neural cells are cultured in serum free conditions, wherein the neural cells are cultured in the presence of fibroblast growth factor 2 (FGF-2); (c) inducing the neural cells to differentiate into differentiated neural cells, wherein the differentiated neural cells are a desired type of differentiated neural cell; and (d) implanting in or administering to the subject the differentiated neural cells. The pluripotent stem cells can be induced pluripotent stem cells produced from a cell of the subject.

Also disclosed are methods of treating a subject, the method comprising (a) culturing neural stem cells, wherein the neural stem cells are cultured on a treated polymer substrate, wherein the neural stem cells are cultured in serum free conditions, wherein the neural stem cells are cultured in the presence of fibroblast growth factor 2 (FGF-2), wherein the neural stem cells were produced by incubating pluripotent stem cells in the presence of an amount of one or more Neural Development Factors (NDF) sufficient to generate cells expressing Pax-6, Otx2, Nestin, or a combination, thereby producing the neural stem cells, wherein the NDF activate the phosphatidylinositol 3-kinase signaling pathway, activate the MAPK signaling pathway, inhibit the TGF-β superfamily signaling pathway, and inhibit the Wnt signaling pathway, wherein the NDF comprise Midkine, Pleiotrophin, insulin-like growth factor-1, or a combination; A83-01, SB431542, or a combination; dorsopmorphin; and PNU-74654, Dickkopf, or a combination; (b) inducing the neural cells to differentiate into differentiated neural cells, wherein the differentiated neural cells are a desired type of differentiated neural cell; and (c) implanting in or administering to the subject the differentiated neural cells. The pluripotent stem cells can be induced pluripotent stem cells produced from a cell of the subject.

Also disclosed are methods of treating a subject, the method comprising (a) inducing neural stem cells to differentiate into differentiated neural cells, wherein the differentiated neural stem cells are a desired type of differentiated neural cell, wherein the neural stem cells were produced by incubating pluripotent stem cells in the presence of an amount of one or more Neural Development Factors (NDF) sufficient to generate cells expressing Pax-6, Otx2, Nestin, or a combination, thereby producing the neural stem cells, wherein the NDF activate the phosphatidylinositol 3-kinase signaling pathway, activate the MAPK signaling pathway, inhibit the TGF-β superfamily signaling pathway, and inhibit the Wnt signaling pathway, wherein the NDF comprise Midkine, Pleiotrophin, insulin-like growth factor-1, or a combination; A83-01, SB431542, or a combination; dorsopmorphin; and PNU-74654, Dickkopf, or a combination; and (b) implanting in or administering to the subject the differentiated neural cells. The pluripotent stem cells can be induced pluripotent stem cells produced from a cell of the subject.

Also disclosed are methods of treating a subject, the method comprising implanting in or administering to the subject differentiated neural cells, wherein the differentiated neural cells were produced by inducing neural stem cells to differentiate into the differentiated neural cells, wherein the neural stem cells were produced by incubating pluripotent stem cells in the presence of an amount of one or more Neural Development Factors (NDF) sufficient to generate cells expressing Pax-6, Otx2, Nestin, or a combination, thereby producing the neural stem cells, wherein the NDF activate the phosphatidylinositol 3-kinase signaling pathway, activate the MAPK signaling pathway, inhibit the TGF-β superfamily signaling pathway, and inhibit the Wnt signaling pathway, wherein the NDF comprise Midkine, Pleiotrophin, insulin-like growth factor-1, or a combination; A83-01, SB431542, or a combination; dorsopmorphin; and PNU-74654, Dickkopf, or a combination, wherein the pluripotent stem cells were induced pluripotent stem cells produced from a cell of the subject. The pluripotent stem cells can be induced pluripotent stem cells produced from a cell of the subject.

### C. Detection of Markers and Identification of Cell Types

The disclosed methods can produce a population of cells in which at least 80%, at least 85%, at least 90%, at least 95%, at least 96%, at least 97%, at least 98%, at least 99%, or 100% of cells are neural stem cells. The disclosed methods can comprise identifying at least 80%, at least 85%, at least 90%, at least 95%, at least 96%, at least 97%, at least 98%, at least 99%, or 100% of cells as neural stem cells. The disclosed methods can comprise identifying cells as neural stem cells, neural cells, and/or differentiated neural cells. The disclosed methods can comprise determining, observing or confirming that at least 80%, at least 85%, at least 90%, at least 95% of cells, at least 96%, at least 97%, at least 98%, at least 99%, or 100%, and identifying at least 80%, at least 85%, at least 90%, at least 95%, at least 96%, at least 97%, at least 98%, at least 99%, or 100% of cells are neural stem cells.

Cell lineage and/or cell-type can be determined by observing cell morphology by, for example, microscopic inspection. The disclosed methods can comprise observing neural stem cell morphology and/or neural cell morphology in the cells generated. Neural cell lineage can be determined by observing neural cell morphology.

Cells generated in the disclosed methods can also be identified through detection of markers, typically cell-surface markers or transcription factors recognized by antibodies. The disclosed methods can comprise detecting the presence of one or more markers, whose presence indicates that the cell is a particular lineage or sublineage, or a particular cell type or sub-type. The skilled person knows markers that may be identified and used as an indication of lineage or cell type.

For example, the disclosed methods can comprise detecting the presence of the marker Pax-6, Otx2, Nestin, or a combination, on the cells and identifying the cells as neural stem cells. Other markers that can be detected later include Sox1, BLBP as a radial glial marker, RIP as an oligodendrocyte marker, OTX2, MITE, ZO-1, or a combination as retinal pigment epithelium markers, and p75, GluR1, synaptophysin, Trks (e.g. TrkA, TrkB, TrkC) and APP, which are present on certain neural cells. Combinations of markers can also be detected. For example, combinations of Pax-6, Otx2, and/or Nestin and be detected.

The disclosed methods can comprise detecting a high percentage of cells expressing neural stem cell markers, for example, at least 80%, at least 85%, at least 90%, at least 95%, at least 96%, at least 97%, at least 98%, at least 99%, or 100% of cells, and/or identifying at least 80%, at least 85%, at least 90%, at least 95%, at least 96%, at least 97%, at least 98%, at least 99%, or 100% of cells as neural stem cells.

Cultures of the disclosed stem cells, neural stem cells, and differentiated neural cells can also be used in drug discovery processes, as well as for testing pharmaceutical compounds for potential unintended activities, as might cause adverse reactions if the compound was administered to a subject or patient. Assessment of the activity of pharmaceutical test compounds generally involves combining the cells with the test compound, determining any resulting change, and then correlating the effect of the compound with the observed change. The screening can be done, for example, either because the compound is designed to have a pharmacological effect on certain cell types, or because a compound designed to have effects elsewhere may have unintended side effects. Two or more drugs (or other test compounds) can also be tested in combination (by combining with the cells either simultaneously or sequentially) to detect possible drug-drug interaction effects. In some applications, compounds are screened initially for potential toxicity. See generally “In vitro Methods in Pharmaceutical Research,” Academic Press, 1997. Cytotoxicity can be determined by the effect on cell viability, survival, morphology, on the expression or release of certain markers, receptors or enzymes, and/or on DNA synthesis or repair, measured by [³H]-thymidine or BrdU incorporation. The cells can be, for example, feeder-free and serum-free.

### EXAMPLES

While crucial progress has recently been made in defining the factors that induce and maintain pluripotency (Takahashi et al. Cell 131:861-872, 2007), knowledge regarding controlled differentiation of hESCs into the earliest emerging precursor cells of the three germ layers (ectoderm, mesoderm, endoderm) remains incomplete even after a decade of research with hESCs (Thomson et al. Science 282:1145-1147, 1998). The application of patient-specific stem cell lines for disease modeling and clinical therapies necessitates mechanistic insights into the early fate choices of pluripotent cells and a better handle on controlled differentiation. Currently, the most widely used methods for differentiating hESCs into neural precursor cells (NPCs) include EB formation as an in vitro model of gastrulation, co-culture with murine stromal cell lines (i.e. PA6, MS5), or high concentrations of the bone morphogenetic protein (BMP) antagonist Noggin (Zhang et al. Nat. Biotechnol 19:1129-1133, 2001; Reubinoff et al. Nat. Biotechnol. 19:1134-1140, 2001; Pera et al. J Cell Sci. 117:1269-1280, 2004; Chambers et al. Nat. Biotechnol. 27:275-280, 2009; Pankratz, M. T. Stem Cells 25:1511-1520, 2007; Pruszak et al. Stem Cells 25:2257-2268, 2007). These empirical differentiation strategies are uncontrolled, generate a heterogeneous mixture of cells, and require cell-sorting strategies to enrich for certain cell populations (Pruszak et al. Stem Cells 25:2257-2268, 2007). Neural induction during early human embryo development and in pluripotent stem cells (which are regarded as models of human embryogenesis) remains poorly understood, and, consequently, a comprehensive strategy that would allow large-scale production exclusively of neural cells has not been developed.

In searching for candidate molecules with potential neural-inducing activity, a new family of developmentally regulated heparin-binding growth and differentiation factors which consists of only two members, pleiotrophin (PTN) and midkine (MK) were focused on. PTN function has recently been studied in hESCs and an anti-apoptotic and survival-promoting effect was reported (Soh et al. Stem Cells 25:3029-3037, 2007). MK expression by hESC has been noted earlier (Dvash et al. Hum. Reprod. 19:2875-2883, 2004; Bendall et al. Nature 448:1015-1021, 2007) but its potential role has not been studied. MK, also called “neurite growth-promoting factor 2”, is a 13-kDa cysteine-rich secreted protein that is highly conserved in evolution and was originally discovered as a retinoic acid (RA)-inducible gene in embryonal carcinoma (EC) cells (Kadomatsu et al. BBRC 151:1312-1318, 1988; Yokota et al. J. Biochem. 123:339-346, 1998). When directly applied to EC cells, MK mimics the effects of RA regarding neural differentiation. In mouse embryos, MK is strongly expressed at sites of epithelial-mesenchymal interactions in various organs and the neural tube reaching maximum expression levels during mid-gestation (hence the name “midkine”). Depending on the biological context, MK can exhibit anti-apoptotic, mitogenic, angiogenic, neurotrophic, and tumorigenic activities. MK is absent or weakly expressed in adult tissues but can be re-expressed by various malignant tumors, after injury, during inflammation and neurodegeneration (Kadomatsu et al. Cancer Lett. 204:127-143, 2004, Stoica et al. JBC 277:35990-35998, 2002). Interestingly, MK is also expressed by Engelbreth-Holm-Swarm tumors and is present in Matrigel (Futaki et al. JBC 278:50691-50701, 2003), a basement membrane matrix that is widely used to attach and culture hESCs and hiPSCs under feeder-free conditions. MK's downstream signaling system activates the PI3K (phosphatidylinositol 3-kinase) pathway followed by MAPK/ERK (mitogen-activated protein kinase/extracellular signal-regulated kinase) (Kadomatsu et al. Cancer Lett. 204:127-143, 2004). The orchestrated balance of these two signaling pathways in concert with inhibition of TGFβ and WNT signaling is pivotal to complete and invariant neural instruction of human pluripotent cells.

### A. Results

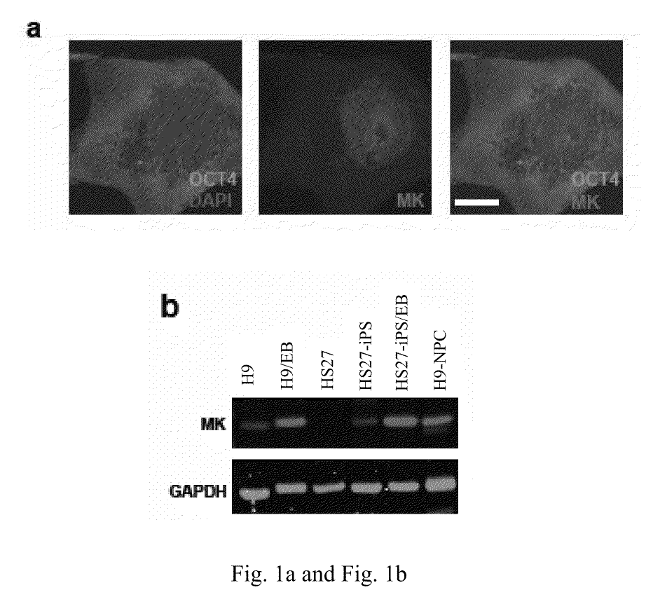

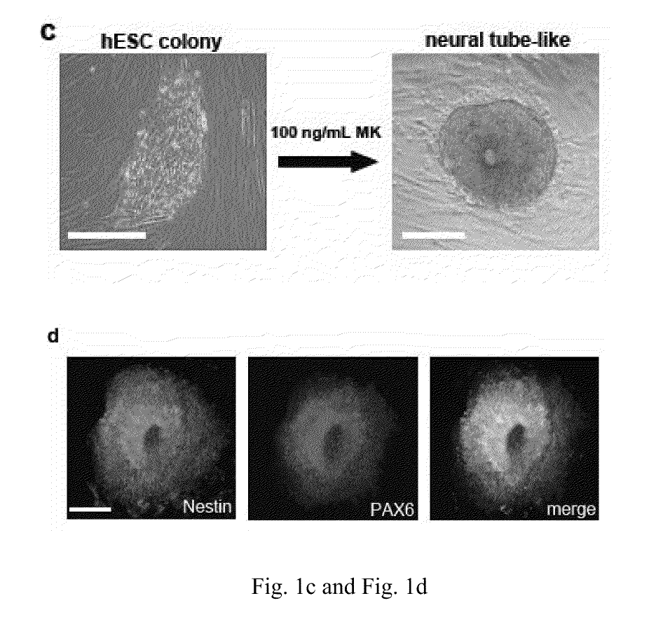

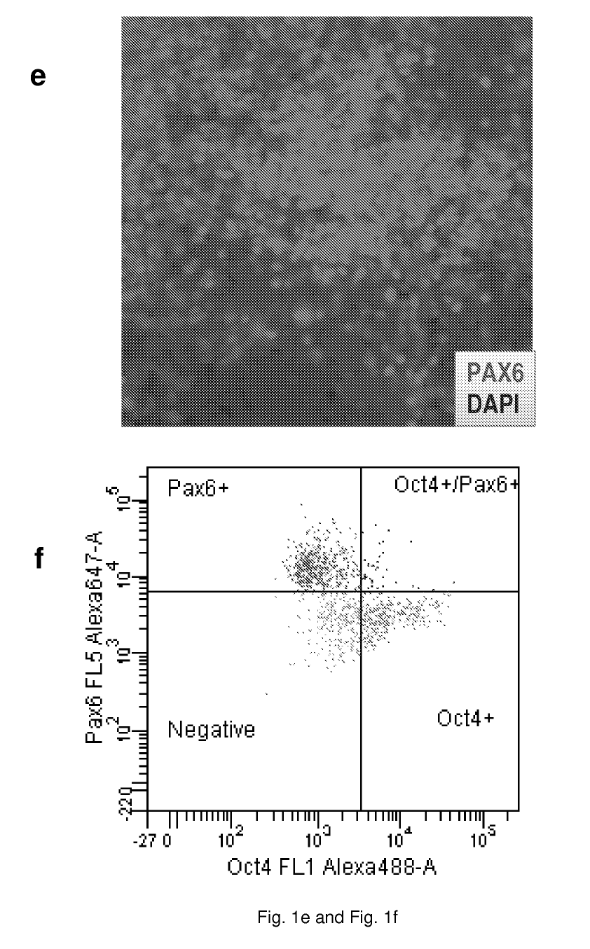

MK was found to be constitutively expressed by a number of widely used hESC cell lines such as H1, H9, H14, BG02, HSF6, and HUES13 (FIGS. 1a, b, FIG. 6 & data not shown). Since native human foreskin fibroblasts (HS27) did not to express MK (FIG. 1b, FIG. 6), the question of if reprogramming of somatic cells, such as these fibroblasts, to pluripotency might induce MK expression was addressed. Following a published protocol (Takahashi et al. Cell 131:861-872, 2007) and using retroviral transduction of the four transcription factors OCT4, SOX2, KLF4, and c-Myc (FIG. 7), 2 hiPSC lines were derived from HS27 fibroblasts. These HS27-hiPSCs as well as other hiPSCs derived from fibroblasts from normal and diseased individuals (Table 1) constitutively expressed MK similar to established hESC lines (FIG. 6). Receptor-type protein tyrosine phosphatase zeta (PTPz) and anaplastic lymphoma kinase (ALK) are part of a receptor complex mediating MK signaling (Kadomatsu et al. Cancer Lett. 204:127-143, 2004; Stoica et al. JBC 277:35990-35998, 2002). Using immunocytochemistry, it was confirmed that both receptors were expressed by hESCs and the newly-generated hiPSCs (FIG. 7). Together, these findings indicate that autocrine MK contributes to the maintenance of the pluripotent state in hESCs and hiPSCs.

TABLE 1
Description and source of hiPSC lines tested in the
present study for neural induction with MK + DAP.

| | | | | iPS cell |
|---|---|---|---|---|
| Fibroblast Type | Source | Order No. | Disease type | line |
| 1. Foreskin (HS27) | ATCC | CRL-1634 | Apparently Healthy | #36.2 |
| 2. Fetal lung | Coriell | AG04432 | Apparently Healthy | #44.1 |
| 3. Adult Skin | Coriell | AG02101 | Apparently Healthy | #45.1 |
| 4. Skin | Coriell | GM03813 | Spinal Muscular | #34.1 |
| | | | Atrophy | |
| 5. Skin | Coriell | GM09677 | Spinal Muscular | #33.2 |
| | | | Atrophy | |
| 6. Skin | Coriell | GM11270 | Rett Syndrome | #32.1 |
| 7. Skin | Coriell | GM17880 | Rett Syndrome | #29.3 |

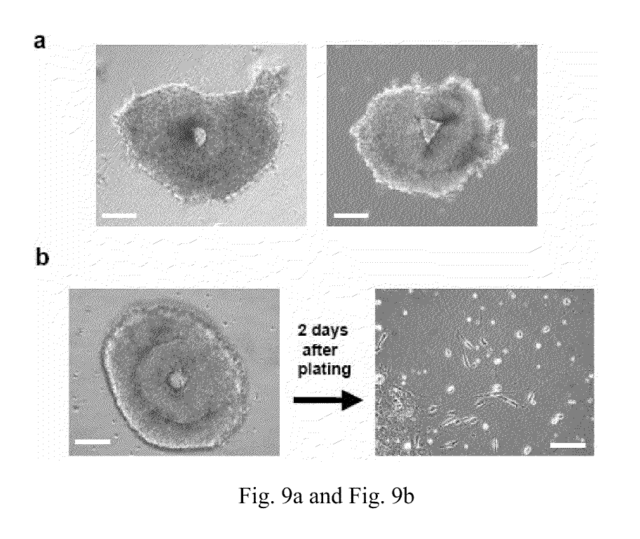

Careful microscopic analysis of spontaneously differentiating hESC colonies revealed that immunoreactivity for MK was consistently increased in regions where Oct4 expression was low or absent indicating an association of MK with differentiation (FIG. 1a). To confirm that increased MK levels correlate with differentiation, EBs from hESCs and HS27-iPSCs were generated and quantitatively analyzed MK expression. Immunoblotting showed that MK was strongly upregulated (6-10 fold) by EBs derived from hESCs and hiPSCs (FIG. 1b). To test if exogenously added MK may display neural-inducing activities, hESCs were cultured in the presence of recombinant MK protein using two different assays. First, manually passaged hESCs (H9 cells) were plated on HS27 fibroblasts and grown in the presence of 100 ng/mL MK. Notably, under these conditions, 5-10% of solitary growing hESC colonies directly differentiated into prominent neural tube-like (NTL) structures within 6-8 days (FIGS. 1c, d and FIG. 9). These NTLs expressed the typical neuroectodermal markers PAX6 and Nestin (FIG. 1d) and, when single NTLs were manually picked under microscopic guidance and attached to laminin-coated dishes, neuronal cells started migrating out from these structures (FIG. 9). These NTL structures, which were interpreted as morphological correlates of neuralization, were never observed when MK was applied together with FGF-2 or when MK was replaced by Notch ligands (delta-like 1, delta-like 4, Jagged1), the BMP antagonist Noggin, insulin-like growth factor 1 (IGF-1) or Forskolin (data not shown). Second, MK-induced differentiation was investigated under feeder-free conditions. Manually passaged H9-hESCs cells were plated on Matrigel and grown in 100 ng/mL MK for 6 days. Using fluorescence-activated cell sorting (FACS) and immunostainings, 20-40% of total cells were found to be differentiated into PAX6⁺ cells (FIGS. 1e, f). Taken together, these experiments indicate that enhanced MK signaling via autocrine upregulation and/or recombinant protein delivery exhibit neural-inducing activity on hESCs.

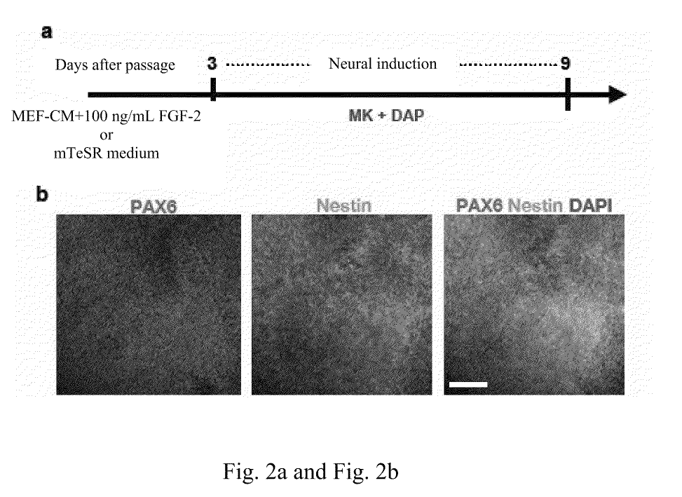

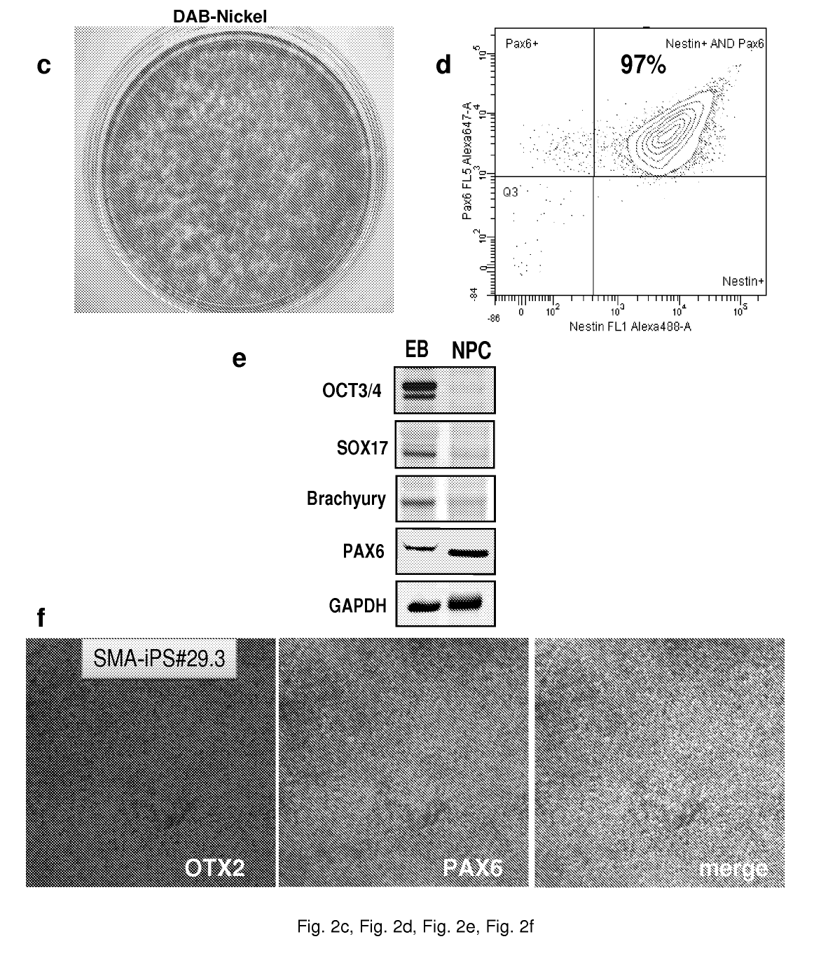

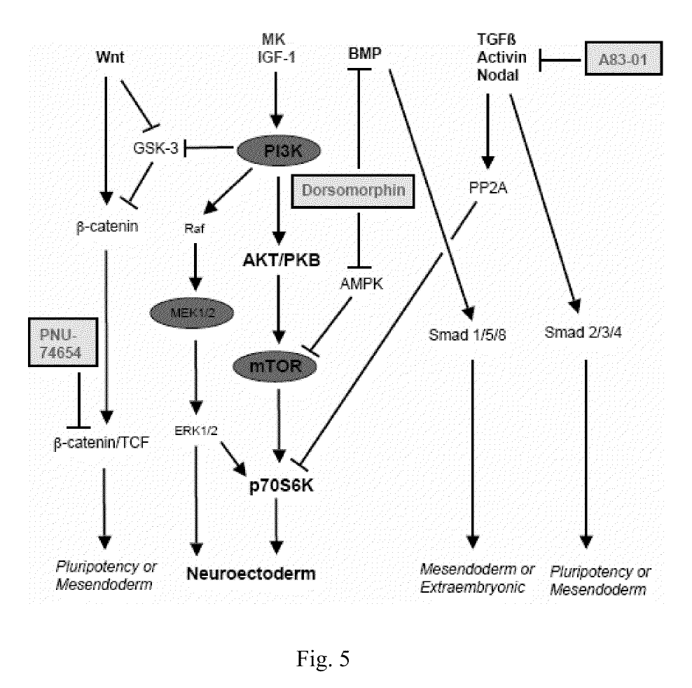

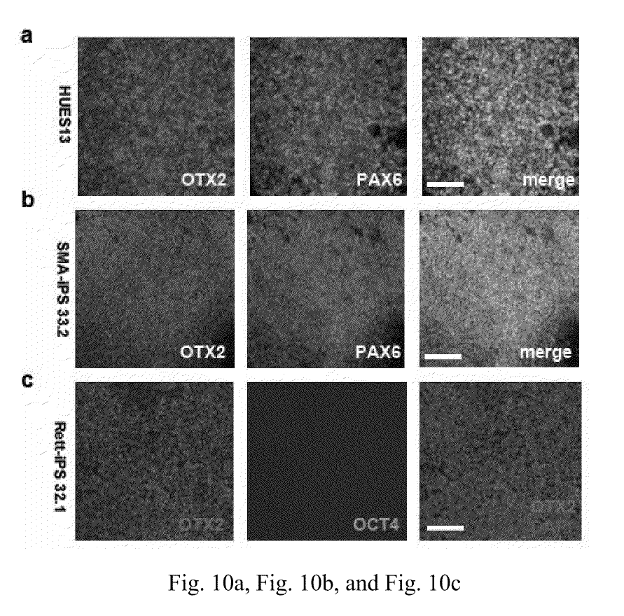

It was next hypothesized that the fraction of cells entering the neural lineage might be increased when applying MK together with small molecules that simultaneously block signaling pathways of self-renewal and non-neural fate choice. Members of the TGFβ superfamily (Activin, Nodal, TGF(3, and BMP) as well as WNT signaling play important roles in maintaining pluripotency and/or promoting mesendoderm differentiation. Specifically, Activin, Nodal, and TGFβ support pluripotent growth while BMP promotes non-neural fate choice of hESCs (Sumi et al. Development 135:2969-2979, 2008, Vallier et al. J. Cell. Sci. 118:4495-4509, 2005; Xu et al. Nat. Methods 2:185-190, 2005; Smith et al. Dev. Biol. 313:107-117, 2008; Xu et al. Cell Stem Cell 3:196-206, 2008). WNT signaling is part of the transcriptional core circuitry that maintains pluripotency, supports the reprogramming process of somatic cells into iPSCs, and antagonizes neural differentiation (Marson et al. Cell Stem Cell 3:132-135, 2008; Aubert et al. Nat. Biotechnol. 20:1240-1245, 2002) (see FIG. 5, Schematic). By using only 2 small molecules, Dorsomorphin (DM) and A81-01, it is possible to block the receptors (Activin receptor-like kinases, ALKs) of all members of the TGFβ superfamily. A81-01 was shown be to be more potent that SB431542 (another inhibitor of ALK 5, 4, 7) and is thereby capable of efficiently blocking the activities of Activin, Nodal, and TGFβ (Tojo et al. Cancer Sci 96:791-800, 2005). DM, also known as Compound C and inhibitor of the AMP-activated protein kinase (AMPK), was recently identified as also a potent inhibitor of BMP receptors (Yu et al. Nat. Chem. Biol. 4:33-41, 2008). In contrast to Noggin, DM can block BMP type I receptors without interfering with their MAPK domain (Yu et al. Nat. Chem. Biol. 4:33-41, 2008). Another advantage of DM over Noggin would be its applicability for large-scale experiments, since recombinant Noggin is required at supraphysiological concentrations (500 ng/mL) for neural differentiation of hESCs and iPSCs (Chambers et al. Nat. Biotechnol. 27:275-280, 2009). WNT signaling can be blocked with the small molecule PNU-74654 disrupting protein-protein interaction of O-catenin and TCF (T cell factor) in the nucleus (Trosset et al. Proteins 64:60-67, 2006). Manually passaged hESCs were first plated on Matrigel and grown for 2 days in the presence of MEF-CM and high FGF-2 (100 ng/mL) or chemically defined mTeSR media. Under these conditions, hESCs and hiPSCs typically recover from the stress of cell passaging. On day 3, differentiation was initiated by switching to a medium consisting of DMEM/F12, 20% Knockout Serum Replacement (KSR), non-essential amino acids, mercapto-ethanol and the cocktail of 100 ng/mL MK and the three inhibitors DM, A 81-01, and PNU-74654 (designated by the acronym “DAP”; 2 μM each) (FIG. 2a). Medium was changed daily and cultures were analyzed on differentiation day 6. Immunocytochemistry for PAX6 and Nestin revealed that virtually every cell in a nearly confluent dish was co-labeled for both proteins (FIGS. 2b, c). This dramatic result was confirmed by quantitative FACS analysis documenting that ˜97% of total cells were co-labeled for PAX6 and Nestin (FIG. 2d). Immunoblotting for various lineage markers demonstrated the high purity of these neural cultures and the absence of non-neural lineages, in contrast to EBs differentiated for 6 days (FIG. 2e). To further characterize the identity of these cells, OTX2 was used as another typical marker of early neuroectodermal cells. In fact, PAX6 and OTX2 expression was largely overlapping in these cultures (FIG. 20, consistent with previous reports indicating that the earliest neuroectodermal NPCs display an anterior forebrain identity (Chambers et al. Nat. Biotechnol. 27:275-280, 2009; Pankratz et al. Stem Cells 25:1511-1520, 2007). The robustness and high efficiency of this neural induction mechanism with various hiPSC lines was confirmed (Table 1, FIG. 2f and FIG. 10). Large and pure populations of PAX6⁺/OTX2⁺/Nestin⁺ neuroectodermal cells were similarly produced in only 6 days from hiPSCs as they had from the hESCs. Time-course experiments showed that OTX2 is the earliest neuroectodermal marker expressed before the onset of PAX6 (FIG. 11). Indeed, the remaining ˜3% of cells that were not yet PAX6-positive were OTX2-positive transitional cells en route to committed NPC lineage (FIG. 11c).

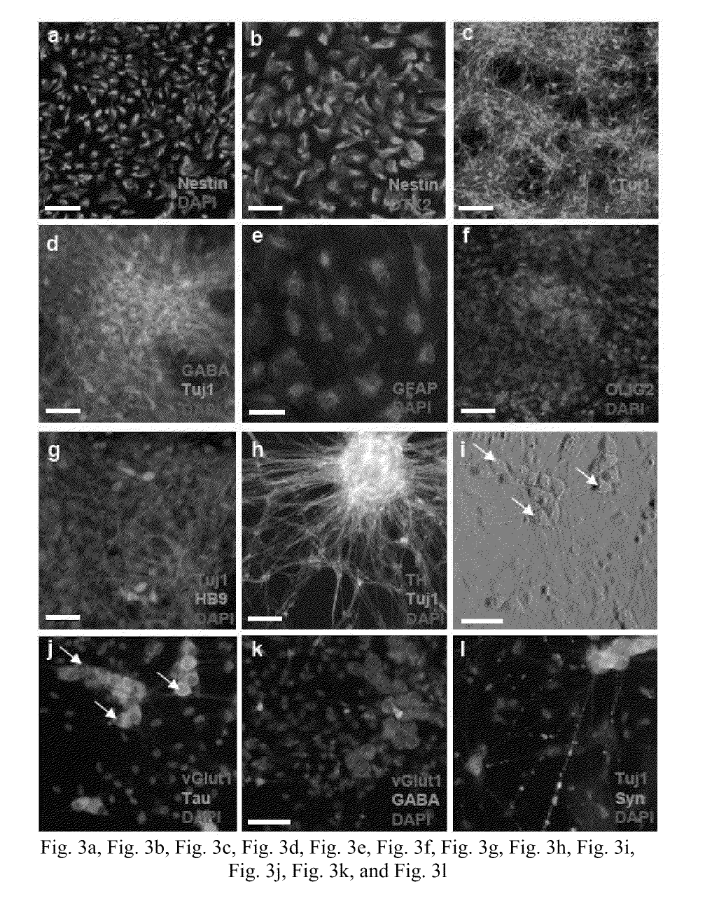

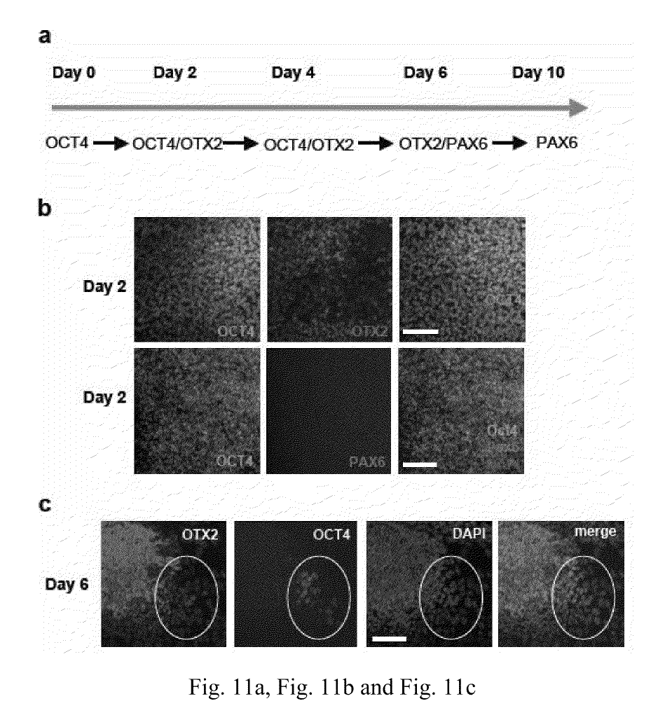

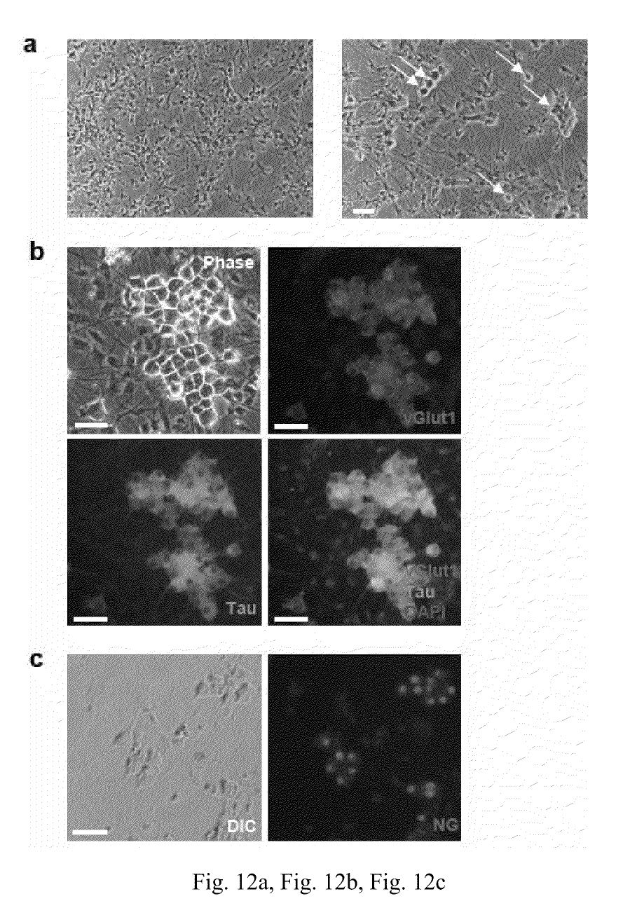

Next, whether these primordial NPCs could be further expanded and differentiated into more mature neural cells was addressed. On day 6, cells derived from hESCs and hiPSCs were passaged and plated on Matrigel-coated dishes. NPCs were grown in DMEM/F12 supplemented with N2B27. Using FGF-2, highly homogeneous and adherently growing Nestin⁺ cell populations could be expanded and cryopreserved (FIGS. 3a, b). Withdrawing FGF-2 and exposure to brain-derived neurotrophic factor (BDNF) and glial-derived neurotrophic factor (GDNF) spontaneously generated neuronal and glial cells as detected with cell-type specific markers (FIG. 3c-e). In order to generate specific neuronal phenotypes, early NPCs (passage 1-2) were exposed to morphogens known to support differentiation into dopamine and motor neurons (MNs) (Chambers et al. Nat. Biotechnol. 27:275-280, 2009). After 1-2 weeks of RA and sonic hedgehog (SHH) treatment, cells expressing OLIG2, an early marker of MN progenitors were obtained (FIG. 30. After applying BDNF, GDNF, and IGF-1 for 2 weeks, the emergence of the MN marker HB9 was noted (FIG. 3 g). To test if the same NPCs could also generate dopamine neurons, cells were exposed to FGF-8 and SHH and further differentiated with BDNF, GDNF, TGF131. Expression of tyrosine hydroxylase (TH), the rate-limiting enzyme of dopamine synthesis, was confirmed with a specific antibody (FIG. 3h). However, most surprisingly, the spontaneous emergence of cells with the typical in vitro appearance of young pyramidal-like neurons was observed when NPCs were briefly expanded in FGF-2 and epidermal growth factor (EGF) and differentiated in BDNF and GDNF (FIG. 3i-1 & FIG. 12). At 1 month, these large phase-bright neuronal cells (soma size 25-45 μM) with prominent nucleoli (FIG. 3i) expressed Tau protein and, importantly, vesicular glutamate transporter (vGLUT1)(FIG. 3j, FIG. 12b). Moreover, these large presumably glutamatergic neurons were devoid of the inhibitory neurotransmitter GABA (FIG. 3k) and expressed the calmodulin-binding protein neurogranin, a marker only expressed by forebrain principal neurons but not interneurons (Singec et al. J. Comp. Neurol. 479:30-42, 2004) (FIG. 12c). At this early stage of differentiation, some neurites of these large neurons were decorated by punctate immunostaining for the presynaptic marker synaptophysin (FIG. 31). Cells resembling such young neocortical pyramidal neurons have been derived only from mouse ESCs (Gaspard et al. Nature 455:351-357, 2008) but not from human pluripotent stem cells. The representative pyramidal-like neurons depicted in FIGS. 3i-1 and FIG. 12 were derived from hiPSCs (line 34.1). Taken together, these experiments indicate not only that primordial NPCs generated by controlled neural induction are responsive to appropriate developmental factors, but also that, if such neural induction comes under experimental control, an array of specific, more mature neuronal cell types from across the rostro-caudal CNS can be generated in a dish.

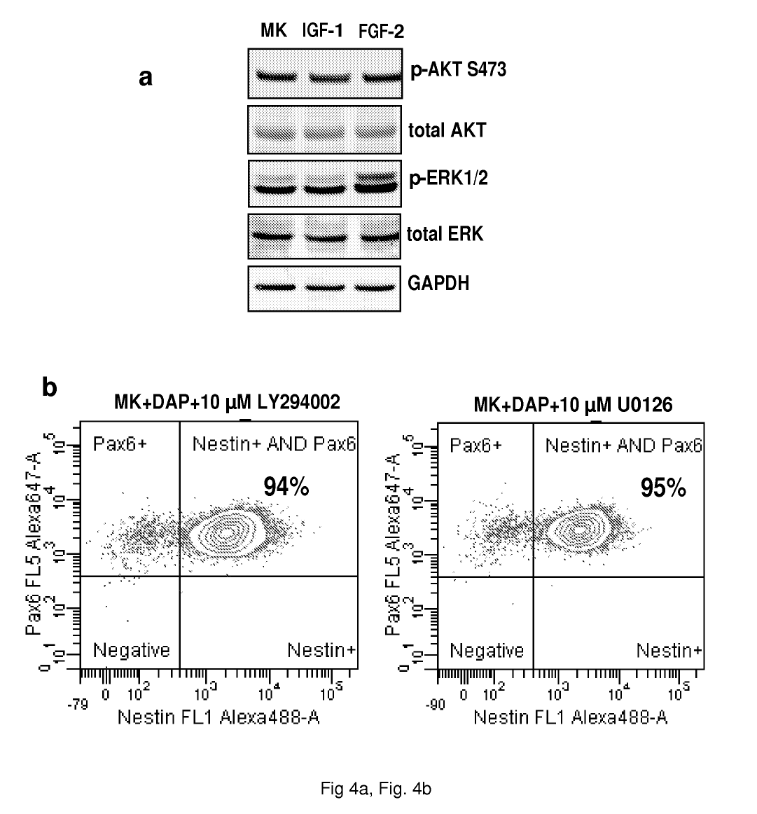

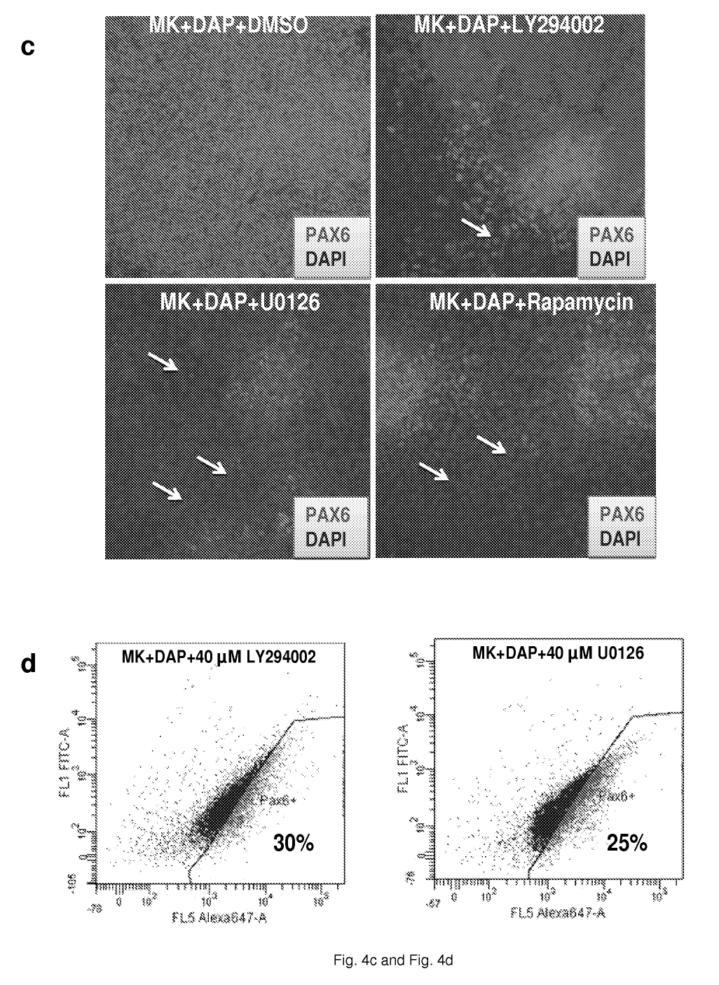

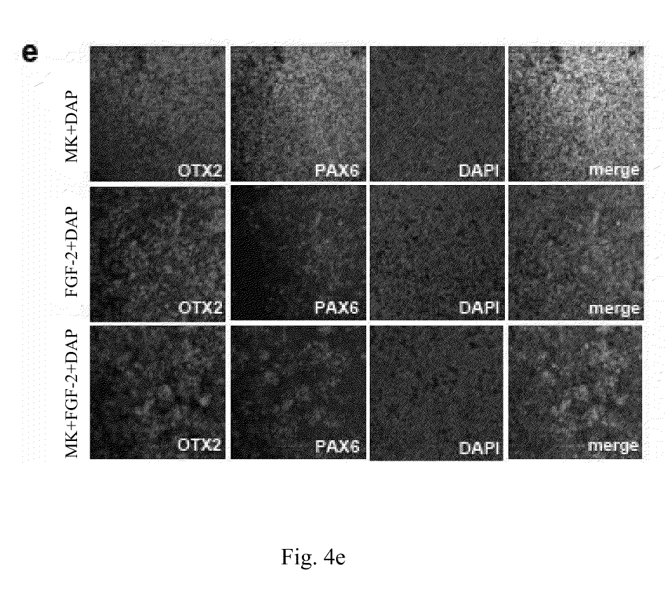

The signal transduction pathway(s) involved in this highly efficient neural conversion was addressed. The PI3K/MAPK pathways were focused on since MK-induced activation of both pathways has been reported (Kadomatsu et al. Cancer Lett. 204:127-143, 2004; Stoica et al. JBC 277:35990-35998, 2002). Since both pathways are already active in undifferentiated hESCs and necessary for their growth (Bendall et al. Nature 448:1015-1021, 2007; Vallier et al. J. Cell. Sci. 118:4495-4509, 2005; Xu et al. Nat. Methods 2:185-190, 2005), cells were exposed to high concentrations (100 ng/mL) of MK, IGF-1, and FGF-2 for 24 hr and phosphorylation levels of AKT and ERK1/2 were compared (FIG. 4a). These experiments indicated that MK as well as IGF-1 signal through both the PI3K pathway and the MAPK pathway; importantly, however, MAPK activation is at a moderate level compared to FGF-2 (FIG. 4a).

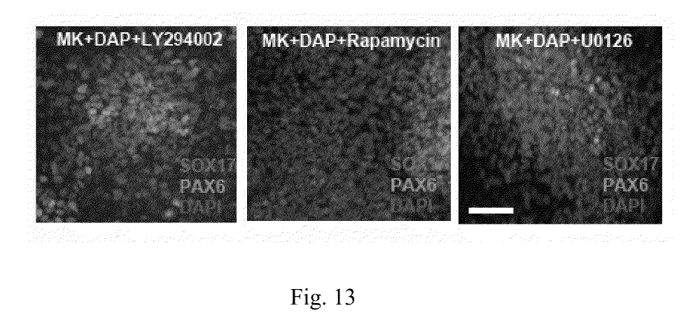

To better understand the role of both pathways in the context of the neural induction process, chemical inhibitors known to block key components of PI3K/MAPK were used. LY294002 is a specific inhibitor of PI3K (McLean et al. Stem Cells 25:29-38, 2007) and Rapamycin selectively blocks the mammalian target of Rapamycin (mTOR), an important downstream kinase of the PI3K pathway (Chen et al. Oncogene 19:3750-3756, 2000). The compound U0126 is a highly selective and potent inhibitor of MAPK signaling by blocking MEK1/2 (Sumi et al. Development 135:2969-2979, 2008; McLean et al. Stem Cells 25:29-38, 2007). These chemical compounds were applied together with 100 ng/mL MK+DAP. The control group received the equivalent volume of DMSO (vehicle) in addition to the cocktail of 100 ng/mL MK+DAP. When using only 10 μM of LY294002 or U0126, significant reductions were not observed in the proportion of NPCs generated from hESCs in response to MK+DAP: ˜95% of total H9 hESCs nevertheless robustly differentiated into PAX6⁺/Nestin⁺ cells as previously observed (compare FIG. 4b with FIG. 2d). However, previous reports indicated that blocking the PI3K pathway in hESCs more efficiently with a higher concentration, 50 μM LY294002, promoted endodermal differentiation (McLean et al. Stem Cells 25:29-38, 2007). Indeed, when 40 μM of either LY294002 or U0126 were used, PAX6 expression in the MK+DAP-treated hESC cultures was dramatically reduced (FIGS. 4c, d). This effect was also obtained when mTOR alone was blocked by even low concentrations of Rapamycin (20-40 nM) (FIG. 4c). Consistent with reports that PI3K pathway blockade supports endodermal differentiation (McLean et al. Stem Cells 25:29-38, 2007; Chen et al. Oncogene 19:3750-3756, 2000), the endodermal marker SOX17 was found to be upregulated in a fraction of PAX6-negative cells when the PI3K or the MAPK pathways were chemically suppressed (FIG. 13). In conclusion, because PAX6 expression could be blocked with any of the 3 inhibitors at similar rates, these experiments indicate that cooperative dual action of the PI3K and MAPK pathways is integral for neural induction.

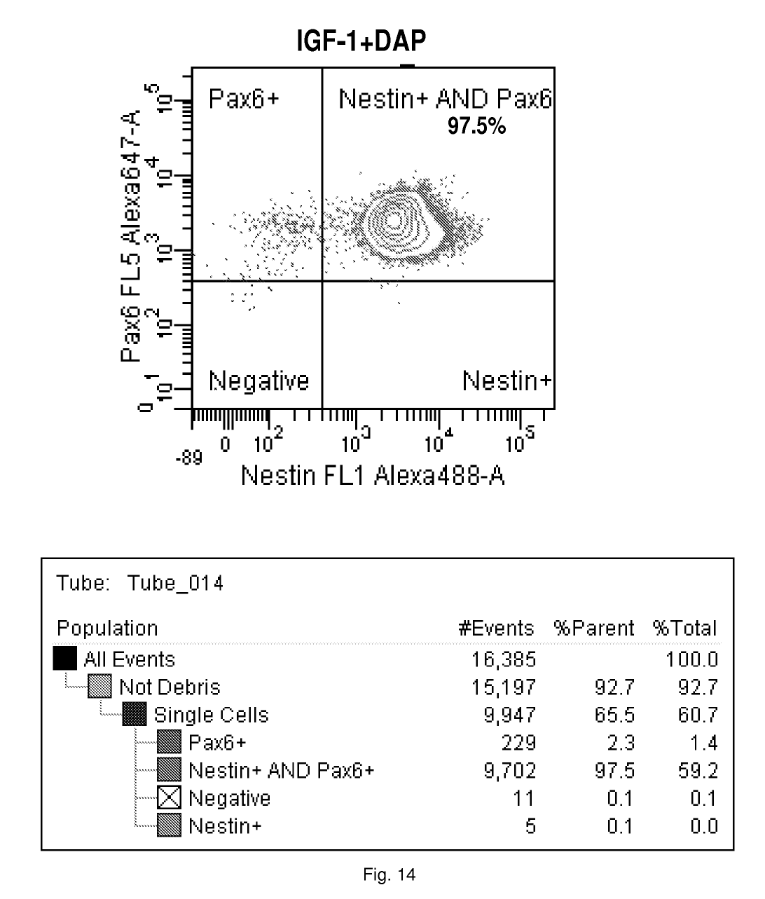

Having established above that modulating the balance between MAPK and PI3K by loss-of-MAPK activity compromised neural induction, it was next determined whether creating an imbalance through excess MAPK activity would affect neuralization. Because FGF-2 is a more potent activator of the MAPK pathway than MK (FIG. 4a), H9 hESCs were treated for 6 days with 100 ng/mL FGF-2+DAP vs. 100 ng/mL MK+DAP (according to FIG. 2a) and compared for OTX2 and PAX6 expression. Importantly, a clear difference in PAX6 expression was found (FIG. 4e). In cultures treated with FGF-2, less abundant PAX6-expressing cells was consistently observed as compared to MK-stimulated cultures. For confirmation, IGF-1—which activates MAPK to a similar degree as MK but less than FGF-2—was added to DAP; high numbers (97.5%) of PAX6-expressing cells were again generated (FIG. 14). These experiments indicated that, under these conditions, both MK and IGF-1 display similar neural differentiation properties and promote PAX6 expression. The MAPK pathway is necessary for neural induction, as shown by chemical inhibition with U0126 (FIGS. 4c, d), but excessive phosphorylation of ERK1/2, as produced by FGF signaling, appears to suppress PAX6 expression (FIGS. 4a, e). The conclusion that FGF-2 impedes neuralization is supported by the finding that MK+FGF-2+DAP also resulted in the generation of low numbers of PAX6-expressing cells (FIG. 4e). Thus, precisely regulated and balanced activation levels of PI3K and MAPK are pivotal parameters during neuralization.

### B. Discussion

Here the key pathway interactions underlying the neural induction of human pluripotent cells have been elucidated. Based on these findings, reliable and uniform differentiate of hESCs and hiPSCs specifically and exclusively into highly pure NPC populations in less than one week is possible. This neural induction paradigm was established by simultaneous activation of these neural-promoting pathways while blocking those that promote pluripotency and non-neural lineage differentiation. Under these conditions, neural induction is not only robust, but also rapid and technically easy, indicating that this method could become a standardized technique in the stem cell field. It is intriguing to note that, unlike previously reported protracted protocols for neural induction, the rapid six day time frame observed approximately minors the emergence of neurectoderm in vivo during human embryo development. Rapidly available populations of NPCs will be instrumental for further dissecting the precise molecular pathways and defining the factors necessary for generating terminally-differentiated neural cells at high purity, including the production of interneurons and pyramidal neurons for modeling human corticogenesis in vitro.

The PI3K and MAPK pathways play important roles during mouse ESC differentiation (Chen et al. Oncogene 19:3750-3756, 2000; Kunath et al. Development 134:2895-902, 2007). In hESCs, signaling through both pathways is key for maintaining the pluripotent state (Bendall et al. Nature 448:1015-1021, 2007; Vallier et al. J. Cell. Sci. 118:4495-4509, 2005). Surprisingly, both PI3K and MAPK pathways were also found to be crucial for human neural lineage entry with requisite concomitant blockade of TGFβ superfamily and WNT signaling (mediators of pluripotency and mesendodermal differentiation). However, equally critical was the balance between MAPK and PI3K activity. Excessive gain-of-MAPK activity (FGF-2) was as potent as loss-of-MAPK activity (U0126) in abrogating neuralization.

The high efficiency of neuralization reported here might also be explained by the fact that DM, A83-01, and PNU-74654 act in multiple synergistic ways with regard to PI3K/MAPK signaling (FIG. 5). DM not only inhibits BMP receptors but also blocks AMPK thereby disinhibiting mTOR (Gwinn et al. Mol. Cell 30:214-226, 2008). Similarly, through inhibition of ALK4, ALK5, and ALK7, A83-01 may indirectly reduce the activity of the phosphatase PP2A and lead to disinhibition of p70S6K, an important downstream kinase of the PI3K pathway (FIG. 5). Activation of PI3K is known to inhibit GSK313 and would, therefore, support WNT signaling. Thus, blocking WNT signaling further downstream at the level of β-catenin/TCF with PNU-74654 can antagonize this effect representing another synergistic component.

Based on the findings presented here, neural induction is a controlled active process involving the upregulation of autocrine factors such as MK and the orchestrated balanced cross-talk between a set of well-defined pathways, the affirmative activation of some and inhibition of others. An instructive feedforward mechanism for neuralization of human cells is proposed rather than the default model that has heretofore been presumed for animal cells (Stern, C. D. Curr. Opin. Cell Biol. 18:692-7, 2006).

### C. Methods

1. Cell Culture and Reagents

Human ESC lines H1, H9, H14, BG02, HSF-6, and HUES13 were cultured on matrigel (BD) or Geltrex (Invitrogen) in the presence of MEF-CM (DMEM/F12, 20% Knockout Serum Replacement, non-essential amino acids, 2-mercaptoethanol). In some experiments, hESCs and iPSCs were cultured in mTeSR medium (Stem Cell Technologies). Neural cells were grown in N2B27 supplement (Gibco). The following reagents were used: MK (Millipore), FGF-2 (NIH/NCI), BDNF (Peprotech), GDNF (Peprotech), FGF-8 (Peprotech), SHH (Peprotech and R&D Systems), Noggin (Peprotech), IGF-1 (Peprotech), DLL1, DLL4, Jagged1 (R&D), all-trans RA (Sigma), Forskolin (Sigma), A 81-01 (Tocris), Dorsomorphin (Sigma), PNU-74654 (Sigma), Rapamycin (Sigma), LY294002 (Tocris), U0126 (Tocris).

2. Human iPS Cell Generation

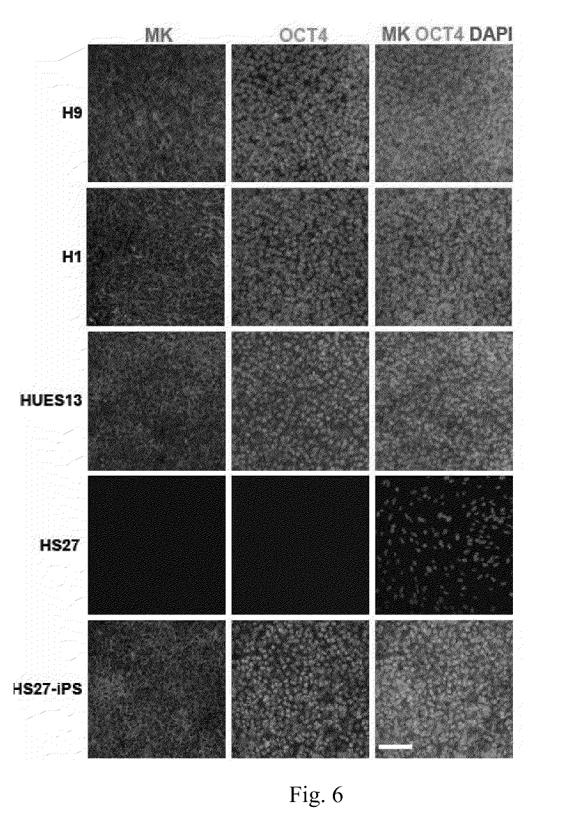

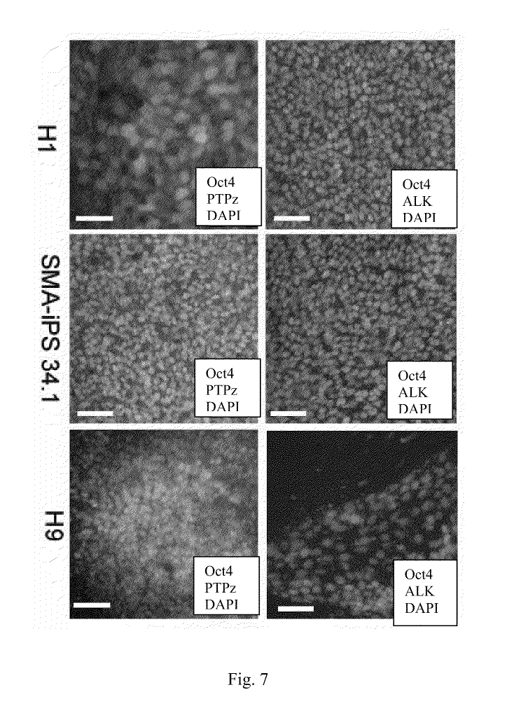

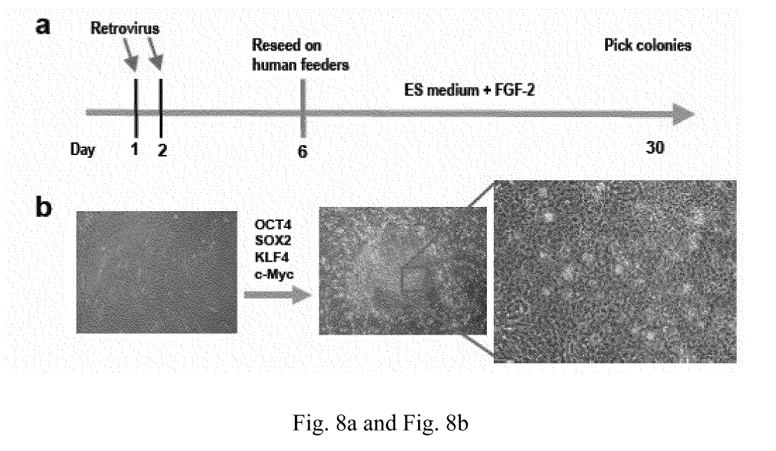

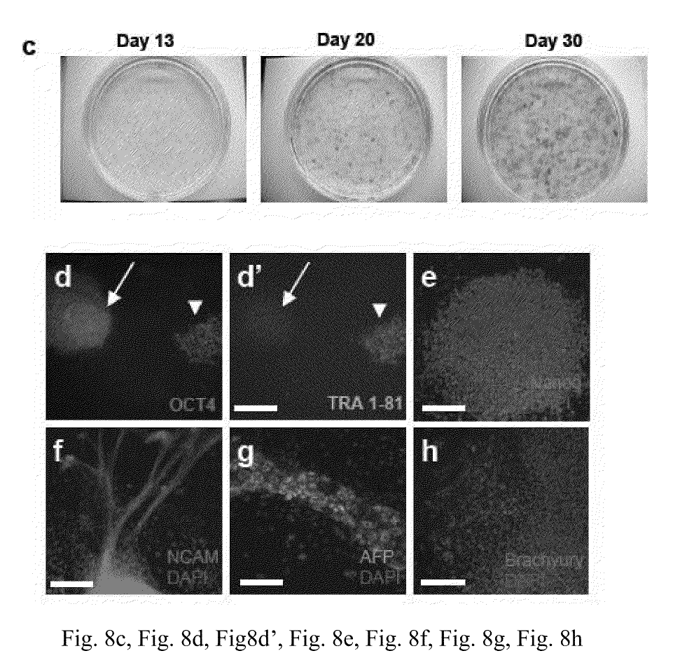

Plasmids expressing OCT4, SOX2, KLF4, and c-MYC were purchased from Addgene. We modified a published retrovirus-based protocol (Takahashi et al. Cell 131:861-872, 2007) in order to generate iPSCs from various human fibroblasts (Table 1). Briefly, we used human HS27 foreskin fibroblasts (ATTC) as feeders instead of MEFs when transduced fibroblasts were re-seeded on day 6 (see also FIG. 8). PLAT-A cells were used to generate retroviral supernatant. The source of the fibroblast cell lines used for reprogramming experiments is detailed in Table 1.

3. Immunocytochemistry

Cells were fixed with 4% paraformaldehyde and stained with following antibodies: anti-mouse OCT-4 (1:200, Santa Cruz, C-10), rabbit Nanog (1:200, Santa Cruz, H-155), mouse TRA 1-81 (1:200, Millipore), alpha-Fetoprotein (1:50, Santa Cruz, AFP-11), goat MK (1:100, R&D Systems), goat SOX17 (1:100, R&D), goat Brachyury (1:100, R&D), PAX6 (1:300, Covance), mouse and rabbit Tuj1 (1:500, Covance), goat OTX2 (1:1000, Neuromics), mouse Nestin (1:400, Millipore), rabbit OLIG2 (1:50, Santa Cruz, H-68), mouse HB9 (1:50, 81.5C10, Developmental Studies Hybridoma Bank), rabbit GFAP (1:1000, DAKO), rabbit and sheep TH (1:500, Pel-Freez), rabbit PTPz (1:50, Santa Cruz), goat ALK (1:50, Santa Cruz). Light-microscopic immunostaining for PAX6 with the DAB method was performed according to the manufacturer (Vector).

4. FACS Analysis

FACS analysis on fixed cells was performed according to Pankratz, M. T. Stem Cells 25:1511-1520, 2007. Permeabilized cells were incubated in primary antibodies (OCT4, PAX6, Nestin) over night. Secondary antibodies were donkey anti-mouse Alexa 488 and donkey anti-rabbit Alexa 647 (Invitrogen). Cells were analyzed on a FACSCanto (BD Biosciences) modified with an ND1 filter in front of the forward scatter photodiode. Alexa 488 was excited 488 nm and emission was detected through a 530/30 bandpass filter. Alexa 647 was excited at 635 nm and emission was detected through a 660/20 bandpass filter. FACSDiVa software (version 5) was used for acquisition and analysis.

5. Western Blot

Cell lysates were prepared in RIPA buffer containing protease and phosphatase inhibitors (Pierce). 4-12% gradient gels (NuPage) were used with Invitrogen's SureLock Mini-Cell and gel-to-membrane transfer was performed with the XCell Blot Module (Invitrogen). The following primary antibodies were used: goat MK (R&D), mouse and rabbit GAPDH (1:1000, Santa Cruz), p-AKT 5473 and total AKT (1:1000, Cell Signaling Technologies), phospho-ERK1/2 and total ERK1/2 (1:1000, Cell Signaling Technologies). The following secondary antibodies were used: donkey anti-mouse Alexa 680 (Invitrogen), donkey anti-rabbit Alexa 680, donkey anti-rabit 800 (Rockland), donkey anti-mouse 800. Western Blots were imaged using the Odyssey Infrared Imaging System (LI-COR).

### REFERENCES

Adams et al., “Tyrosine Kinase and c-Jun NH2-Terminal Kinase Mediate Hypertrophic Responses to Prostaglandin F2α in Cultured Neonatal Rat Ventricular Myocytes,” Circ. Res. 83:167-78 (1998).
Anderson et al., “Requirement for integration of signals from two distinct phosphorylation pathways for activation of MAP kinase,” Nature, 1990, 343, 651.
Arrighi et al. “A Critical Role for p38 Mitogen-Activated Protein Kinase in the Maturation of Human Blood-Derived Dendritic Cells Induced by Lipopolysaccharide, TNF-α, and Contact Sensitizers,” The Journal of Immunology, 2001, 166: 3837-3845.
Aubert, J., Dunstan, H., Chambers, I. & Smith, A. Functional gene screening in embryonic stem cells implicates Wnt antagonism in neural differentiation. Nat. Biotechnol. 20, 1240-1245 (2002).
Bacus et al., “Taxol-induced apoptosis depends on MAP kinase pathways (ERK and p38) and is independent of p53,” Oncogene 2001; 20, 147-155.
Bendall, S. C. et al. IGF and FGF cooperatively establish the regulatory stem cell niche of pluripotent human cells in vitro. Nature 448, 1015-1021 (2007).
Bjorbaek et al., “Divergent Functional Roles for p90rsk Kinase Domains,” J. Biol. Chem. 1995, 270; 18848).
Bogoyevitch et al., “Stimulation of the Stress-Activated Mitogen-Activated Protein Kinase Subfamilies in Perfused Heart: p38/RK Mitogen-Activated Protein Kinases and c-Jun N-Terminal Kinases Are Activated by Ischemia/Reperfusion,” Circ. Res. 79:162-73 (1996).
Burgess et al., “Regulation of the c-jun Gene in p210 BCR-ABL Transformed Cells Corresponds With Activity of JNK, the c-jun N-Terminal Kinase,” Blood 92:2450-60 (1998).
Chambers, S. M. et al. Highly efficient neural conversion of human ES and iPS cells by dual inhibition of SMAD signaling. Nat. Biotechnol. 27, 275-280 (2009).
Chen et al., “Phosphorylation of the c-Fos transrepression domain by mitogen-activated protein kinase and 90-kDa ribosomal S6 kinase,” Proc. Natl. Acad. Sci. USA 1993, 90; 10952.
Chen, Y., Li, X., Eswarakumar, V. P., Seger, R. & Lonai, P. Fibroblast growth factor (FGF) signaling through PI3-kinase and Akt/PKB is required for embryoid body differentiation. Oncogene 19, 3750-3756 (2000).
Crews et al., “The primary structure of MEK, a protein kinase that phosphorylates the ERK gene product,” Science 1992, 258, 478.
Ding et al., “Cross-talk between signaling pathways and the multidrug resistant protein MDR-1,” Brit. J. Cancer 2001, 85: 1175-1184.
Dvash, T. et al. Temporal gene expression during differentiation of human embryonic stem cells and embryoid bodies. Hum. Reprod. 19, 2875-2883 (2004).
Force et al., “Stress-Activated Protein Kinases in Cardiovascular Disease,” Circ. Res. 78:947-53 (1996).
Frey and Mulder, “Involvement of Extracellular Signal-regulated Kinase 2 and Stress-activated Protein Kinase/Jun N-Terminal Kinase Activation by Transforming Growth Factor β in the Negative Growth Control of Breast Cancer Cells,” Cancer Res. 1997, 57; 628.
Futaki, S. et al. Molecular basis of constitutive production of basement membrane components. Gene expression profiles of Engelbreth-Holm-Swarm tumor and F9 embryonal carcinoma cells. J. Biol. Chem. 278, 50691-50701 (2003).
Gaspard, N. et al. An intrinsic mechanism of corticogenesis from embryonic stem cells. Nature 455, 351-357 (2008).
Gwinn, D. M. et al. AMPK phosphorylation of raptor mediates a metabolic checkpoint. Mol. Cell 30, 214-226 (2008).
Kadomatsu, K. & Muramatsu, T. Midkine and pleiotrophin in neural development and cancer. Cancer Lett. 204, 127-143 (2004).
Kadomatsu, K., Tomomura, M. & Muramatsu, T. cDNA cloning and sequencing of a new gene intensely expressed in early differentiation stages of embryonal carcinoma cells and in mid-gestation period of mouse embryogenesis. Biochem. Biophys. Res. Commun. 151, 1312-1318 (1988).
Kempiak et al., “The Jun Kinase Cascade Is Responsible for Activating the CD28 Response Element of the IL-2 Promoter: Proof of Cross-Talk with the LκB Kinase Cascade,'J. Immunol. 162:3176-87 (1999).
Kim et al. “Angiotensin Blockade Inhibits Activation of Mitogen-Activated Protein Kinases in Rat Balloon-Injured Artery,” Circulation 97:1731-7 (1998).
Kunath, T. et al. FGF stimulation of the Erk1/2 signalling cascade triggers transition of pluripotent embryonic stem cells from self-renewal to lineage commitment. Development 134, 2895-902 (2007).
Liang et al. “Mechanical Strain Increases Expression of the Brain Natriuretic Peptide Gene in Rat Cardiac Myocytes,” J. Biol. Chem. 272:28050-6 (1997).
Lowry, W. E. et al. Generation of human induced pluripotent stem cells from dermal fibroblasts. Proc. Natl. Acad. Sci. USA 105, 2883-2888 (2008).
MacKeigan et al., “MEK inhibition enhances paclitaxel-induced tumor apoptosis” J. Biol Chem. 2000 Dec. 15; 275(50):38953-6.
Marson, A. et al. Wnt signaling promotes reprogramming of somatic cells to pluripotency. Cell Stem Cell 3, 132-135 (2008).
McDaid et al., “Selective Potentiation of Paclitaxel (Taxol)-Induced Cell Death by Mitogen-Activated Protein Kinase Kinase Inhibition in Human Cancer Cell Lines MoI Pharmacol.” 2001 August; 60(2):290-301.
McLean, A. B. et al. Activin A efficiently specifies definitive endoderm from human embryonic stem cells only when phosphatidylinositol 3-kinase signaling is suppressed. Stem Cells 25, 29-38 (2007).
Mercedes Rincón et al, “The JNK and p38 MAP kinase signaling pathways in T-cells mediated immune response Free Radical Biology and Medicine, Volume 28, Issue 9, 1 May 2000, Pages 1328-1337.
Moodie et al., “Complexes of Ras.GTP with Raf-1 and mitogen-activated protein kinase kinase,” Science 1993, 260; 1658.
Nishina et al, “Impaired CD28-mediated Interleukin 2 Production and Proliferation in Stress Kinase SAPK/ERK1 Kinase (SEK1)/Mitogen-activated Protein Kinase Kinase 4 (MKK4)-deficient T Lymphocytes,” J. Exp. Med. 186:941-53 (1997).
Oliver et al., Proc. Soc. Exp. Biol. Med. 1995, 210; 162.
Othsuka et al., “Synergistic induction of tumor cell apoptosis by death receptor antibody and chemotherapy agent through JNK/p38 and mitochondrial death pathway” Oncogene 2003:22, 2034-2044.
Panka et al., “Targeting the Mitogen-Activated Protein Kinase Pathway in the Treatment of Malignant Melanoma” Clin Cancer Res. 2006 Apr. 1: 2371s-2375s.
Pankratz, M. T. Directed neural differentiation of human embryonic stem cells via an obligated primitive anterior stage. Stem Cells 25, 1511-1520 (2007).
Pera, M. F. et al. Regulation of human embryonic stem cell differentiation by BMP-2 and its antagonist noggin. J. Cell Sci. 117, 1269-1280 (2004).
Pruszak, J., Sonntag, K. C., Aung, M. H., Sanchez-Pernaute, R. & Isacson, O. Markers and methods for cell sorting of human embryonic stem cell-derived neural cell populations. Stem Cells 25, 2257-2268 (2007).
Raingeaud et al., “MKK3- and MKK6-regulated gene expression is mediated by the p38 mitogen-activated protein kinase signal transduction pathway,” Mol. Cell Biol. 1996, 16; 1247).
Reubinoff, B. E. et al. Neural progenitors from human embryonic stem cells. Nat Biotechnol. 19, 1134-1140 (2001).
Rouse et al., “A novel kinase cascade triggered by stress and heat shock that stimulates MAPKAP kinase-2 and phosphorylation of the small heat shock proteins,” Cell 1994, 78; 1027)
Sánchez-Pérez et al. “CL100/MKP-1 modulates JNK activation and apoptosis in response to cisplatin” Oncogene 2000, 19; 5142-5152
Singec, I., Knoth, R., Ditter, M., Volk, B. & Frotscher, M. Neurogranin is expressed by principal cells but not interneurons in the rodent and monkey neocortex and hippocampus. J. Comp. Neurol. 479, 30-42 (2004).
Sivaraman et al., “Hyperexpression of mitogen-activated protein kinase in human breast cancer,” J. Clin. Invest. 1997, 99; 1478
Smith, J. R. et al Inhibition of Activin/Nodal signaling promotes specification of human embryonic stem cells into neuroectoderm. Dev. Biol. 313, 107-117 (2008).
Soh, B. S. et al. Pleiotrophin enhances clonal growth and long-term expansion of human embryonic stem cells. Stem Cells 25, 3029-3037 (2007).
Stem, C. D. Neural induction: 10 years on since the ‘default model’. Cum Opin. Cell Biol. 18, 692-7 (2006).
Stoica, G. E. et al. Midkine binds to anaplastic lymphoma kinase (ALK) and acts as a growth factor for different cell types. J. Biol. Chem. 277, 35990-35998 (2002).
Sumi, T., Tsuneyoshi, N., Nakatsuji, N. & Suemori, H. Defining early lineage specification of human embryonic stem cells by the orchestrated balance of canonical Wnt/beta-catenin, Activin/Nodal and BMP signaling. Development 135, 2969-2979 (2008).
Takahashi, K. et al. Induction of pluripotent stem cells from adult human fibroblasts by defined factors. Cell 131, 861-872 (2007).
Thomson, J. A. et al. Embryonic stem cell lines derived from human blastocysts. Science 282, 1145-1147 (1998).
Tojo, M. et al. The ALK-5 inhibitor A-83-01 inhibits Smad signaling and epithelial-to-mesenchymal transition by transforming growth factor-beta. Cancer Sci. 96, 791-800 (2005).
Trosset, J. Y. et al. Inhibition of protein-protein interactions: the discovery of druglike beta-catenin inhibitors by combining virtual and biophysical screening. Proteins 64, 60-67 (2006).
Vallier, L., Alexander, M. & Pedersen, R. A. Activin/Nodal and FGF pathways cooperate to maintain pluripotency of human embryonic stem cells. J. Cell. Sci. 118, 4495-4509 (2005).
van Sventer et al., “Pyk2 is differentially regulated by beta 1 integrin- and CD28-mediated co-stimulation in human CD4+T lymphocytes,” Eur. J. Immunol. 28:3867-77 (1998).
Whelchel et al, “Inhibition of ERK activation attenuates endothelin-stimulated airway smooth muscle cell proliferation,” Am. J. Respir. Cell Mol. Biol. 1997, 16; 589).
Widakowich et al. “Review; Side Effects of Approved molecular Targeted Therapies in Solid Cancers,” The Oncologist, Vol. 12, No. 12, 1443-1455, December 2007).
Wilson et al., “Jnk, but not MAPK, activation is associated with Fas-mediated apoptosis in human T cells,” Eur. J. Immunol. 26:989-94 (1996).
Xu et al. “Acute hypertension activates mitogen-activated protein kinases in arterial wall,” J. Clin. Invest. 97:508-14 (1996)).
Xu et al., “Constitutively activated JNK is associated with HTLV-1 mediated tumorigenesis,” Oncogene 13:135-42 (1996).
Xu, R. H. et al. Basic FGF and suppression of BMP signaling sustain undifferentiated proliferation of human ES cells. Nat. Methods 2, 185-190 (2005).
Xu, R. H. et al. NANOG is a direct target of TGFbeta/activin-mediated SMAD signaling in human ESCs. Cell Stem Cell 3, 196-206 (2008).
Yokota, C. et al. Midkine counteracts the activin signal in mesoderm induction and promotes neural formation. J. Biochem. 123, 339-346 (1998).
Yu, P. B. et al. Dorsomorphin inhibits BMP signals required for embryogenesis and iron metabolism. Nat. Chem. Biol. 4, 33-41 (2008).
Zhang, S. C., Wernig, M., Duncan, I. D., Brtistle, O. & Thomson, J. A. (2001). In vitro differentiation of transplantable neural precursors from human embryonic stem cells. Nat. Biotechnol. 19, 1129-1133 (2001).

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a stem cell” includes a plurality of such stem cells, reference to “the stem cell” is a reference to one or more stem cells and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps.

Those skilled in the art will recognize, or be able to ascertain using no more than routine experimentation, many equivalents to the specific embodiments of the method and compositions described herein. Such equivalents are intended to be encompassed by the following claims.

1. A method of producing neural cells, the method comprising incubating pluripotent stem cells in the presence of an amount of one or more Neural Development Factors (NDF) sufficient to generate cells expressing Pax-6, Otx2, Nestin, or a combination, thereby producing neural cells.

2. The method of claim 1, wherein the NDF activate the phosphatidylinositol 3-kinase (PI3K) signaling pathway.

3. The method of claim 1, wherein the NDF activate the mitogen-activated protein kinase (MAPK) signaling pathway.

4. The method of claim 1, wherein the NDF activate the phosphatidylinositol 3-kinase signaling pathway and the mitogen-activated protein kinase (MAPK) signaling pathway in a balanced manner.

5. The method of claim 1, wherein the NDF inhibit the TGF-β superfamily signaling pathway.

6. The method of claim 1, wherein the NDF inhibit the Wnt signaling pathway.

7. The method of claim 1, wherein the NDF activate the phosphatidylinositol 3-kinase signaling pathway, inhibit the TGF-β superfamily signaling pathway, and inhibit the Wnt signaling pathway.

8. The method of claim 1, wherein the NDF activate the phosphatidylinositol 3-kinase signaling pathway, activate the MAPK signaling pathway, inhibit the TGF-β superfamily signaling pathway, and inhibit the Wnt signaling pathway.

9. The method of claim 1, wherein the Wnt signaling pathway is inhibited after the β-catenin destruction complex.

10. The method of claim 1, wherein the NDF comprise an activator of the phosphatidylinositol 3-kinase signaling pathway.

11. The method of claim 1, wherein the NDF comprise an activator of the MAPK signaling pathway.

12. The method of claim 1, wherein the NDF comprise Midkine, Pleiotrophin, insulin-like growth factor-1, or a combination.

13. The method of claim 1, wherein the NDF comprise an activator of the phosphatidylinositol 3-kinase signaling pathway and the MAPK signaling pathway.

14. The method of claim 1, wherein the NDF comprise Midkine, insulin-like growth factor-1, or a combination.

15. The method of claim 1, wherein the NDF comprise Midkine.

16. The method of claim 1, wherein the NDF comprise an activator of the phosphatidylinositol 3-kinase signaling pathway and an activator of the MAPK signaling pathway.

17. The method of claim 1, wherein the NDF comprise an inhibitor of the TGF-β superfamily signaling pathway.

18. The method of claim 1, wherein the NDF comprise A83-01, SB431542, or a combination.

19. The method of claim 1, wherein the NDF comprise dorsomorphin.

20. The method of claim 1, wherein the NDF comprise dorsomorphin and A83-01.

21. The method of claim 1, wherein the NDF comprise an inhibitor of the Wnt signaling pathway.

22. The method of claim 1, wherein the NDF comprise PNU-74654, Dickkopf, or a combination.

23. The method of claim 1, wherein the NDF comprise an activator of the phosphatidylinositol 3-kinase signaling pathway, an inhibitor of the TGF-β superfamily signaling pathway, and an inhibitor of the Wnt signaling pathway.

24. The method of claim 1, wherein the NDF comprise Midkine, Pleiotrophin, insulin-like growth factor-1, or a combination; A83-01, SB431542, or a combination; dorsopmorphin; and PNU-74654, Dickkopf, or a combination.

25. The method of claim 1, wherein the NDF comprise Midkine, A83-01, dorsopmorphin, and PNU-74654.

26. The method of claim 1, wherein the NDF comprise insulin-like growth factor-1, A83-01, dorsopmorphin, and PNU-74654.

27. The method of claim 1, wherein the NDF activate tyrosine kinase anaplastic lymphoma kinase (ALK).

28. The method of claim 1, wherein the NDF activate insulin-like growth factor (IGF) receptor.

29. The method of claim 1, wherein the NDF activate phosphatidylinositol 3-kinase (PI3K).

30. The method of claim 1, wherein the NDF inhibit Activin receptor-like kinase 5 (ALK5).

31. The method of claim 1, wherein the NDF inhibit Activin receptor-like kinase 4 (ALK4).

32. The method of claim 1, wherein the NDF inhibit Activin receptor-like kinase 7 (ALK7).

33. The method of claim 1, wherein the NDF inhibit ALK5, ALK4, and ALK7.

34. The method of claim 1, wherein the NDF inhibit protein phosphatase 2A (PP2A).

35. The method of claim 1, wherein the NDF inhibit Bone morphogenic protein (BMP) receptor.

36. The method of claim 1, wherein the NDF inhibit adenosine monophosphate-activated protein kinase (AMPK).

37. The method of claim 1, wherein the NDF inhibit interaction between β-catenin and T cell factor (TCF).

38. The method of claim 1, wherein the NDF activate protein-tyrosine phosphatasζ (PTPζ).

39. The method of claim 1, wherein the NDF inhibit SMAD1, SMAD5, SMAD8, or a combination.

40. The method of claim 1, wherein the NDF inhibit SMAD2, SMAD3, SNAD4, or a combination.

41. The method of claim 1, wherein the NDF inhibit Wnt binding to Frizzled.

42. The method of claim 1, wherein the NDF inhibit lipoprotein receptor-related protein (LRP) binding to Frizzled.

43. The method of claim 1, wherein the NDF inhibit β-catenin stabilization.

44. The method of claim 1, wherein the NDF inhibit β-catenin binding to T cell factor (TCT).

45. The method of claim 1, wherein the NDF activate insulin-like growth factor-1 receptor (IGF-1R).

46. The method of claim 1, wherein the NDF activate insulin receptor substrate-1 (IRS-1).

47. The method of claim 1, wherein prior to incubating in the presence of the NDF, the stem cells are cultured in the absence of feeder cells and on an extracellular matrix.

48. The method of claim 47, wherein the extracellular matrix is Matrigel™.

49. The method of claim 1, wherein prior to incubating in the presence of the NDF the stem cells are cultured on fibroblasts.

50. The method of claim 49, wherein the fibroblasts are from the same species as the stem cells.

51. The method of claim 49, wherein the fibroblasts are from the same subject as the stem cells.

52. The method of claim 49, wherein the fibroblasts are human fibroblasts.

53. The method of claim 1, wherein prior to incubating in the presence of the NDF, the stem cells are cultured in the presence of fibroblast growth factor 2 (FGF-2).

54. The method of claim 53, wherein prior to incubating in the presence of the NDF, at the same time as incubating in the presence of the NDF is initiated, or during incubating in the presence the NDF the use of FGF-2 is discontinued.

55. The method of claim 54, wherein the use of FGF-2 is discontinued at the same time as incubating in the presence of the NDF is initiated.

56. The method of claim 54, wherein the use of FGF-2 is discontinued at the same time as incubating in the presence of the NDF is initiated by replacing growth medium containing FGF-2 and lacking NDF with growth medium lacking FGF-2 and containing the NDF.

57. The method of claim 1, wherein prior to incubating in the presence of the NDF, at least a portion of the stem cells are cultured to a density of 1×10⁴ cells per square centimeter or greater.

58. The method of claim 1, further comprising culturing the neural cells.

59. The method of claim 58, wherein the neural cells are cultured on a treated polymer substrate.

60. The method of claim 59, wherein the treated polymer substrate is CELLBIND™, or substrate treated with Matrigel™, Geltrex™, or fibronectin.

61. The method of claim 58, wherein the neural cells are cultured in serum free conditions and N2 supplement.

62. The method of claim 58, wherein the neural cells are cultured in the presence of fibroblast growth factor 2 (FGF-2) and epidermal growth factor (EGF).

63. The method of claim 58, wherein the neural cells are passaged with Accutase.

64. The method of claim 1, wherein the stem cells are human stem cells.

65. The method of claim 1, wherein the stem cells are embryonic stem cells (ESC).

66. The method of claim 1, wherein the stem cells are derived from embryonic or fetal tissue.

67. The method of claim 1, wherein the stem cells are derived from postfetal tissue.

68. The method of claim 1, wherein the stem cells are derived from adult tissue.

69. The method of claim 1, wherein the stem cells are derived from differentiated tissue.

70. The method of claim 1, wherein the stem cells are induced pluripotent stem cells (iPSC).

71. The method of claim 1, wherein the stem cells are derived from a subject in need of neural cells.

72. The method of claim 1, wherein the neural cells are neural stem cells.

73. The method of any one of claims 1-71, wherein the neural cells form neural tube-like structures.

74. The method of claim 1 further comprising differentiating the neural cells into differentiated neural cells.

75. The method of claim 1, wherein the neural cells are differentiated neural cells.

76. The method of claim 1, wherein the neural cells comprise neurons, astrocytes, oligodendrocytes, or a combination.

77. The method of claim 1, wherein the neural cells comprise pyramidal neurons, motor neurons, spinal ventral horn motor neurons, neurons of the ventral mesencephalon, interneurons, glial cells, radial glial cells, retinal pigment epithelium, oligodendrocytes, dopamine neurons, GABA neurons, glutamate neurons, catecholinergic neurons, serotoninergic neurons, cholinergic neurons, or a combination.

78. The method of claim 1, wherein the neural cells comprise pyramidal neurons.

79. The method of claim 1, wherein the neural cells comprise dopamine neurons.

80. The method of claim 1, wherein the neural cells comprise motor neurons.

81. A neural cell produced by the method of claim 1.

82. A neural cell produced by the method comprising incubating pluripotent stem cells in the presence of an amount of one or more Neural Development Factors (NDF) sufficient to generate cells expressing Pax-6, Otx2, Nestin, or a combination.

83. A method of treating a subject, the method comprising administering a neural cell produced by the method of claim 1.

84. A method of treating a subject, the method comprising incubating pluripotent stem cells in the presence of an amount of one or more Neural Development Factors (NDF) sufficient to generate cells expressing Pax-6, Otx2, Nestin, or a combination, thereby producing neural cells, and
administering one or more of the neural cells to the subject.

85. The method of claim 83, wherein the stem cell is from the same species as the subject.

86. The method of claim 83, wherein the stem cell is from the subject.

87. A method of detecting a state or characteristic of a cell, the method comprising detecting the state or characteristic in a neural cell produced by the method of claim 1.

88. A method of testing conditions for differentiation of neural stem cells, the method comprising exposing a neural cell produced by the method of claim 1 to test conditions and determining if the neural stem cells differentiate into a cell type of interest.

89. The method of claim 88, wherein the cell type of interest is neuron, astrocyte, oligodendrocyte, or a combination.

90. A method of producing neural cells, the method comprising incubating pluripotent stem cells in the presence of an amount of one or more Neural Development Factors (NDF) sufficient to generate cells expressing Pax-6, Otx2, Nestin, or a combination, thereby producing neural cells,
wherein the NDF activate the phosphatidylinositol 3-kinase signaling pathway, inhibit the TGF-β superfamily signaling pathway, and inhibit the Wnt signaling pathway,
wherein the NDF comprise Midkine, Pleiotrophin, insulin-like growth factor-1, or a combination; A83-01, SB431542, or a combination; dorsopmorphin; and PNU-74654, Dickkopf, or a combination.

91. A method of producing neural cells, the method comprising
(a) culturing pluripotent stem cells in the presence of fibroblast growth factor 2 (FGF-2);
(b) incubating the stem cells in the presence of an amount of one or more Neural Development Factors (NDF) sufficient to generate cells expressing Pax-6, Otx2, Nestin, or a combination, thereby producing neural cells,
wherein the NDF activate the phosphatidylinositol 3-kinase signaling pathway, inhibit the TGF-β superfamily signaling pathway, and inhibit the Wnt signaling pathway,
wherein the NDF comprise Midkine, Pleiotrophin, insulin-like growth factor-1, or a combination; A83-01, SB431542, or a combination; dorsopmorphin; and PNU-74654, Dickkopf, or a combination,
wherein prior to incubating in the presence of the NDF, at the same time as incubating in the presence of the NDF is initiated, or during incubating in the presence the NDF the use of FGF-2 is discontinued;
(c) culturing the neural cells, wherein the neural cells are cultured on a treated polymer substrate, wherein the neural cells are cultured in serum free conditions, wherein the neural cells are cultured in the presence of fibroblast growth factor 2 (FGF-2) and epidermal growth factor (EGF).

92. The method of claim 91, wherein the pluripotent stem cells are cultured in conditioned medium or chemically defined medium.

93. The method of claim 92, wherein the medium is mouse embryonic fibroblast-conditioned media (MEF-CM), mTeSR™ or StemPro™.

94. The method of claim 91, wherein the treated polymer substrate is CELLBIND™, or substrate treated with Matrigel™, Geltrex™, or fibronectin.

95. The method of claim 91, wherein the serum free conditions comprise N2 supplement and B27 supplement.

96. The method of claim 91, wherein the NDF comprise Midkine, A83-01, dorsopmorphin, and PNU-74654.

97. The method of claim 91, wherein the NDF comprise insulin-like growth factor-1, A83-01, dorsopmorphin, and PNU-74654.
